(19) United States
(12) Patent Application Publication
(10) Pub. No.: US 2022/0196671 A1
(43) Pub. Date: Jun. 23, 2022

(54) Methods for Identification of Scrambled Disulfides in Biomolecules

(71) Applicant: Regeneron Pharmaceuticals, Inc., Tarrytown, NY (US)

(72) Inventors: Andrew Kleinberg, Roslyn Heights, NY (US); Yuan Mao, Hartsdale, NY (US)

(21) Appl. No.: 17/554,784

(22) Filed: Dec. 17, 2021

Related U.S. Application Data
(60) Provisional application No. 63/128,146, filed on Dec. 20, 2020.

(51) Int. Cl. G01N 33/68; B01D 15/16; B01D 15/30; G01N 30/72; G01N 30/06

(52) U.S. Cl. CPC G01N 33/6848; B01D 15/166; B01D 15/305; G01N 2030/8831; G01N 33/6857; G01N 30/06; G01N 30/7233

(57) ABSTRACT

Disclosed are methods for identification of one or more non-native disulfide bonds in a biomolecule (e.g, an antibody). In an example, a method includes performing a digestion of the biomolecule under non-reducing conditions to provide a sample comprising a plurality of biomolecule fragments, contacting the sample to a separation column, applying a first mobile phase gradient comprising trifluoroacetic acid (TFA) and a small molecule additive to the separation column, applying a second mobile phase gradient comprising TFA in acetonitrile (ACN) and a small molecule additive to the separation column, performing a partial reduction procedure on the eluted sample, applying the partially reduced eluted sample components to a mass spectrometer, and performing a mass spectrometric analysis on the partially reduced eluted sample components to identify the one or more non-native disulfide bonds in the biomolecule.

### CROSS REFERENCE TO RELATED APPLICATION

This application claims the benefit under 35 USC § 119(e) of U.S. Provisional Application No. 63,128,146, Dec. 20, 2020, which is incorporated herein by reference in its entirety for all purposes.

### REFERENCE TO A SEQUENCE LISTING

This application incorporates by reference the Sequence Listing submitted in Computer Readable Form as file 10870US01-Sequence, created on Dec. 17, 2021 and containing 12,466 bytes.

### TECHNICAL FIELD

Embodiments herein pertain to mass spectral analyses, and, more specifically, to methods for improving an ability to use mass spectral analysis to identify low abundance scrambled disulfides in biomolecules.

### BACKGROUND

Disulfide bonds are present in a large number of proteins (nearly one-third) in the eukaryotic proteome. The formation of disulfide bonds involves reaction between sulfhydryl (SH) side chains of two cysteine residues. Native disulfide bond formation acts to stabilize proteins, and disulfide bonds are important for effective protein functionality.

Therapeutic monoclonal antibodies can bind to specific epitopes on cell surface receptors or other biological targets, and the efficacy and stability of such therapeutic antibodies is dependent on proper formation of native disulfide bonds. Alternatively, the formation of non-native (e.g., scrambled) disulfide bonds in proteins, including but not limited to therapeutic monoclonal antibodies, can lead to destabilization, improper folding, aggregate formation and an inability of the particular protein to function effectively. Thus, elucidation of the presence of scrambled disulfides in proteins, for example therapeutic antibodies, is of importance. Current challenges to determining the presence of disulfide scrambling in proteins include low abundance, and hence difficulty in detecting disulfide scrambling when relying on methodology such as mass spectral analyses. Discussed herein are methods for improving an ability to use mass spectral analysis to identify low abundance scrambled disulfides in biomolecules such as therapeutic monoclonal antibodies.

### BRIEF SUMMARY OF THE INVENTION

In one aspect, the present invention provides a method for identification of one or more non-native disulfide bonds in a biomolecule. The method comprises performing a digestion of the biomolecule under non-reducing conditions to yield a sample that includes a plurality of fragments of the biomolecule; contacting the sample to a separation column under conditions that permit sample components to bind to a column substrate; applying a first mobile phase gradient to the separation column, wherein the first mobile phase gradient comprises trifluoroacetic acid (TFA) and a small molecule additive at a concentration of about 1-2 mM; applying a second mobile phase gradient to the separation column, wherein the second mobile phase gradient comprises TFA in acetonitrile (ACN) and a small molecule additive at the concentration of about 1-2 mM; performing a partial reduction procedure via treatment of eluted sample components with tris(2-carboxyethyl)phosphine (TCEP) at a concentration of 10-100 μM; applying the partially reduced eluted sample components to a mass spectrometer; and performing a mass spectrometric analysis on the partially reduced eluted sample components to identify the one or more non-native disulfide bonds in the biomolecule.

In some embodiments, the small molecule additive in the first mobile phase is glycine.

In some embodiments, the small molecule additive in the first mobile phase is glycine and the glycine concentration is about 1 mM.

In some embodiments, the small molecule additive in the first mobile phase is glycine and the glycine concentration is about 2 mM.

In some embodiments, the small molecule additive in the second mobile phase is glycine and the glycine concentration is about 1 mM.

In some embodiments, the small molecule additive in the second mobile phase is glycine and the glycine concentration is about 2 mM.

In some embodiments, the small molecule additive in one or more of the first mobile phase and the second mobile phase is selected from alanine, serine, valine, N-acetyl glycine, methionine, β-alanine, aspartic acid, or N-methyl glycine.

In some embodiments, TFA concentration in the first mobile phase is about 0.05% to 0.1% TFA in H₂O.

In some embodiments, TFA concentration in the second mobile phase comprises about 0.05% TFA in 80% ACN and 20% H₂O or about 0.1% TFA in 80% ACN and 20% H₂O.

In some embodiments, the biomolecule is a monoclonal antibody of isotype IgG1, IgG2, IgG3, IgG4, or mixed isotype.

In some embodiments, the biomolecule is recombinantly produced.

In some embodiments, the partial reduction procedure is conducted for a duration of 500 ms-3 s.

In some embodiments, performing the digestion of the biomolecule comprises performing a denaturation and alkylation step to yield a denatured alkylated biomolecule, performing a pre-digestion step on the denatured alkylated biomolecule to yield a predigested denatured alkylated biomolecule, and performing a digestion step on the predigested denatured alkylated biomolecule following the pre-digestion step to yield the sample that is contacted to the separation column.

In some embodiments, the denaturation and alkylation step includes denaturing the biomolecule in 7-9M urea in the presence of an alkylating agent at a pH of about 5.5-5.9. In some cases, the denaturation and alkylation step is conducted at a temperature between 45-55° C. In some cases, the alkylating agent is N-ethyl maleimide (NEM) at a concentration between 5-15 mM. In some cases, the alkylating agent is iodo-acetamide (IAM) at a concentration of about 0.5-5 mM. In some cases, the method includes performing the denaturation and alkylation step for 20-40 minutes.

In some embodiments, performing the pre-digestion step includes incubating the denatured alkylated biomolecule in the presence of recombinant Lys-C protease at a pH between 5-5.6. In some cases, the pre-digestion step is performed at a temperature between 35-40° C. In some cases, the pre-digestion step is performed for a duration between 30 minutes to 90 minutes. In some cases, a ratio of recombinant Lys-C protease to the denatured alkylated biomolecule is between 1:5 and 1:20, respectively.

In some embodiments, performing the digestion step includes incubating the predigested denatured alkylated biomolecule in the presence of recombinant Lys-C protease and trypsin protease at a pH between 5-5.6. In some cases, a ratio of recombinant Lys-C protease to the predigested denatured alkylated biomolecule during the digestion step is between about 1:5 and about 1:20, respectively. In some cases, a ratio of trypsin protease to the predigested denatured alkylated biomolecule is between about 1:2 and about 1:10, respectively. In some cases, the digestion step is performed at a temperature between 35-40° C. In some cases, the digestion step is performed for 2-4 hours.

In some embodiments, the partially reduced eluted sample components include one or more disulfide peptides and corresponding reduced partner peptides. In some cases, each of the one or more disulfide peptides and corresponding reduced partner peptides enter into the mass spectrometer at a same time. In some cases, the mass spectrometer is a tandem mass spectrometer, and performing the mass spectrometric analysis includes obtaining a MS1 spectra and a MS2 spectra. In some cases, a parallel reaction monitoring (PRM) inclusion list is built with the corresponding reduced partner peptides. In some cases, a disulfide identification confidence score is assigned for the one or more disulfide peptides that is based on a confidence scoring system.

In some embodiments, the confidence scoring system comprises steps of indicating whether a MS1 mass of a disulfide peptide is identified via the mass spectrometric analysis; indicating whether a MS1 mass of a first reduced partner peptide corresponding to the disulfide peptide is identified via the mass spectrometric analysis; indicating whether a MS1 mass of a second reduced partner peptide corresponding to the disulfide peptide is identified via the mass spectrometric analysis; indicating whether a MS2 mass of the first reduced partner peptide is identified with a score greater than a predetermined threshold; and/or indicating whether a MS2 mass of the second reduced partner peptide is identified with a score greater than the predetermined threshold; for each of the indicating steps of the confidence scoring system, assigning a single point where the corresponding peptide is identified and no points where the corresponding peptide is not identified; summing the single points; and assigning the disulfide identification confidence score based on the summing, where the greater the sum, the higher the confidence.

In some embodiments, performing the mass spectrometric analysis comprises determining a disulfide scrambling percentage for each of the one or more non-native disulfide bonds in the biomolecule. In examples, the disulfide scrambling percentage is a ratio of an average peak area of a peptide that includes a non-native disulfide bond to a sum of the average peak area of the peptide that includes the non-native disulfide bond plus another average peak area of two peptides that include native disulfide bonds corresponding to cysteine residues that are involved in the non-native disulfide bond.

In some embodiments, the concentration of TCEP is between 20 μM and 80 μM.

In some embodiments, the concentration of TCEP is about 40 μM.

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges and all values falling within such ranges are encompassed within the scope of the present disclosure. Each of the values discussed above or herein may be expressed with a variation of 1%, 5%, 10% or 20%. For example, a concentration of 10 mM may be expressed as 10 mM±0.1 mM (1% variation), 10 mM±0.5 mM (5% variation), 10 mM±1 mM (10% variation) or 10 mM±2 mM (20% variation). Other embodiments will become apparent from a review of the ensuing detailed description.

### BRIEF DESCRIPTION OF THE FIGURES

Embodiments will be readily understood by the following detailed description in conjunction with the accompanying drawings and the appended claims. Embodiments are illustrated by way of example and not by way of limitation in the figures of the accompanying drawings.

FIG. 1A depicts an illustrative example of native disulfide bonds and non-native disulfide bonds in a biomolecule (e.g., therapeutic monoclonal antibody).

FIG. 1B depicts an illustrative example scheme of how disulfide scrambling occurs in a biomolecule.

FIG. 2 depicts total ion current (TIC) and an extracted ion chromatograph (EIC) for a disulfide formed from GPSVFPLAPCSR (SEQ ID NO: 1) and TYTCNVDHKPSNTK (SEQ ID NO: 2) as analyzed via liquid chromatography mass spectrometry (LC-MS), and tandem mass spectrometry analysis illustrating an ability to detect peptide fragments corresponding to GPSVFPLAPCSR (SEQ ID NO: 1) but not TYTCNVDHKPSNTK (SEQ ID NO: 2).

FIG. 3 depicts illustrative LC-MS analysis of a trypsin-digested monoclonal antibody (mAb) treated with varying concentrations of tris(2-carboxyethyl)phosphine (TCEP). mAb was digested with trypsin under non-reduced conditions to maintain disulfide connections, following separation via high performance liquid chromatography (HPLC) at a flow rate of 50 ul/min (0.02% TFA and 0.08% FA). Following separation, column eluents were treated with varying concentrations of TCEP (0 mM, 0.4 mM, 0.8 mM, 2 mM and 4 mM) and NH₄OH (final concentration 0.12%) before being analyzed via mass spectrometry. The TCEP and NH₄OH was added to eluent using a mixing tee at a flow rate of 2 μL/min. Data is depicted as counts vs. mass-to-charge ratio (m/z). Shown is the disulfide and corresponding reduce peptides (R1 and R2) at varying TCEP concentrations.

FIG. 4 is a graph showing MS signal of a peptide fragment VVSVLTVLHQDWLNGK (SEQ ID NO: 3) corresponding to mAb1 as a function of TCEP, NH₄OH and glycine concentration. Depicted are five conditions including control (no TCEP, no NH₄OH, no glycine), sample 1 (2 mM TCEP, 0.12% NH₄OH), sample 2 (2 mM TCEP, 0.12% NH₄OH, 2 mM glycine), sample 3 (2 mM TCEP, 2 mM glycine, no NH₄OH), and sample 4 (2 mM glycine only). As compared to control, 2 mM TCEP in the presence of 0.12% NH₄OH causes a decrease in MS signal (sample 1). Inclusion of 2 mM glycine to samples containing 2 mM TCEP and 0.12% NH₄OH provides marginal improvement (sample 2) over the conditions of sample 1. 2 mM TCEP and 2 mM glycine in absence of NH₄OH (sample 3) similarly provides only marginal improvement over the conditions of sample 1 and sample 2. Samples containing 2 mM glycine alone (sample 4) show approximately a 10× signal boost as compared to samples containing TCEP and glycine. For each of the samples illustrated at FIG. 4 conditions included mAb1 at a loading amount of 0.1 μg with 0.05% TFA in the mobile phase.

FIGS. 5A and 5B depict MS signal of two peptide fragments VVSVLTVLHQDWLNGK (SEQ ID NO: 3) (FIG. 5A) and DTLMISR (SEQ ID NO: 4) (FIG. 5B) corresponding to mAb1 as a function of TCEP concentration. Sample conditions included mAb1 at a loading amount of 0.1 μg, 0.05% TFA and 2 mM glycine, treated with varying concentrations of TCEP (0 μM, 20 μM, 40 μM, 80 μM, 200 μM, 400 μM, 800 μM and 2000 μM). As depicted, MS signal decreases with increasing concentrations of TCEP.

FIGS. 6A and 6B depict MS signal of peptide fragments generated from trypsin digestion of mAb1 in the presence of varying concentrations of TCEP. FIG. 6A illustrates MS signal for a disulfide corresponding to peptides NQVSLTCLVK (SEQ ID NO: 5) and WQQGNVFSCSVMHEALHNHYTQK (SEQ ID NO: 6), and FIG. 6B depicts MS signal for the corresponding individual reduced peptides. Sample conditions included mAb1 at a loading amount of 1 μg, 0.05% TFA and 2 mM glycine, treated with the indicated TCEP concentrations.

FIGS. 7A and 7B illustrate relative abundance of peptide fragments generated from trypsin digestion of mAb2 without reduction with TCEP (FIG. 7A) and after partial reduction with TCEP at 40 μM (FIG. 7B). For each of FIGS. 7A-7B, reduced peptides correspond to GPSVFPLAPCSR (SEQ ID NO: 1) and STSESTAALGCLVK (SEQ ID NO: 7), and which in non-reduced form comprise the disulfide peptide. HPLC eluent was mixed with 40 μM TCEP to induce partial reduction of disulfides, along with the addition of 2 mM glycine to boost MS signal by 10-20× as compared to samples lacking glycine. The partial reduction methodology post-elution results in disulfide peptides entering into the mass spectrometer at a same time as reduced partner peptides.

FIGS. 8A, 8B, 8C, 8D, 8E, 8F and 8G depict relative abundance of peptide fragments generated from trypsin digestion of mAb2 illustrating MS signal of disulfide peptides and reduced partner peptides as a function of small molecule additive (e.g., glycine) and/or ion pairing agents (e.g., TFA or FA) in the mobile phase. For each of FIGS. 8A-8E, reduced partner peptides correspond to GPSVFPLAPCSR (SEQ ID NO: 1) and TYTCNVDHKPSNTK (SEQ ID NO: 2), which in non-reduced form comprise the disulfide peptide. For each of FIGS. 8F-8G, reduced partner peptides correspond to GPSVFPLAPCSR (SEQ ID NO: 1) and STSESTAALGCLVK (SEQ ID NO: 7), which in non-reduced form comprise the disulfide. Sample conditions for each of FIGS. 8A-8G included 5 μg of digested mAb2 and 40 μM TCEP, with FIG. 8A including 0.05% TFA without glycine, FIG. 8B including 2 mM glycine and 0.05% TFA, FIG. 8C including 0.1% FA without glycine, FIG. 8D including 0.1% FA without glycine, FIG. 8E including 2 mM glycine and 0.1% FA, FIG. 8F including 0.1% FA without glycine, and FIG. 8G including 2 mM glycine and 0.1% FA.

FIGS. 9A and 9B depict MS signal for peptide fragments generated from trypsin digestion of mAb2. Shown at FIGS. 9A-9B are reduced partner peptides GPSVFPLAPCSR (SEQ ID NO: 1) and STSESTAALGCLVK (SEQ ID NO: 7), which in their non-reduced form comprise the disulfide peptide. Conditions depicted at each of FIGS. 9A-9B include the following: 40 μM TCEP, 2 mM glycine and 0.05% TFA; 2000 μM TCEP, 0.12% NH₄OH and 0.05% TFA; and 2000 μM TCEP, 0.12% NH₄OH and 0.1% FA. FIG. 9B depicts the same data as that depicted in FIG. 9A, with a zoomed-in y-axis to illustrate MS signal improvement with 40 μM TCEP and 2 mM glycine. mAb2 loading amount for each of FIGS. 9A-9B was 5 ug.

FIGS. 10A and 10B depict MS signal for peptide fragments generated from trypsin digestion of mAb2. Shown at FIGS. 10A-10B is data corresponding to reduced partner peptides GPSVFPLAPCSR (SEQ ID NO: 1), labeled as R1, and TYTCNVDHKPSNTK (SEQ ID NO: 2), labeled as R2, which in their non-reduced form comprise the disulfide peptide as shown. FIG. 10A depicts relative abundance of the disulfide and corresponding reduced partner peptides. FIG. 10B depicts m/z for a second stage of mass spectrometry (MS2), in which ions from the first stage (MS1) are selectively fragmented to generate the MS2 spectra. For FIGS. 10A-10B, sample conditions included mAb2 at a loading amount of 5 μg, 40 μM TCEP, 2 mM glycine and 0.05% TFA.

FIG. 10C illustrates an example mAb that contains 16 unique cysteine residues.

FIG. 11 depicts MS/MS spectra of a disulfide and corresponding reduced partner peptides GPSVFPLAPCSR (SEQ ID NO: 1) and STSESTAALGCLVK (SEQ ID NO: 7). Peptide fragments were generated from trypsin digestion of mAb2. The data depicted at FIG. 11 illustrates that MS/MS spectra of disulfides contain a large degree of complexity as compared to corresponding reduced partner peptides, and that performing a post-column partial reduction with 40 μM TCEP enables simpler characterization of any detectable scrambled disulfides.

FIG. 12 depicts cysteine-containing peptides generated from trypsin digestion of mAb2. Shown for reference is the cysteine residue number and indication of whether the cysteine is located on the heavy (H) or light (L) chain of mAb2. Tryptic-peptides include LSCAGSGFTFR (SEQ ID NO: 8), AEDTAVYYCAK (SEQ ID NO: 9), GPSVFPLAPCSR (SEQ ID NO: 1), STSESTAALGCLVK (SEQ ID NO: 7), TYTCNVDHKPSNTK (SEQ ID NO: 2), TPEVTCVVVDVSQEDPEVQFNVWYVDGVEVHNAK (SEQ ID NO: 10), CK, NQVSLTCLVK (SEQ ID NO: 37), WQEGNVFSCSVMHEALHNHYTQK (SEQ ID NO: 11), DIVMTQSPLSLPVTPGEPASISCR (SEQ ID NO: 12), VEAEDVGFYYCMQALQTPYTFGQGTK (SEQ ID NO: 13), SGTASVVCLLNNFYPR (SEQ ID NO: 14), VYACEVTHQGLSSPVTK (SEQ ID NO: 15), and SFNRGEC (SEQ ID NO: 16).

FIG. 13 illustrates all possible scrambled disulfide connections from mAb2, with the exception of the hinge region peptide YGPPCPPCPAPEFLGGPSVFLFPPKPK (SEQ ID NO: 46). Hinge region peptides are excluded because such peptides contain more than one cysteine (e.g., 2), thus forming more than one disulfide bond and hence, a scrambled version of this peptide would be expected to be very large and complex. Tandem mass spectrometry (MS/MS) using a targeted MS2 approach as herein disclosed was used to identify scrambled disulfide connections. 5 μg of trypsin-digested mAb2 was separated and partially reduced using 40 μM TCEP. 2 mM glycine was used to boost MS signal. Each disulfide connection is coded according to confidence level in the determination (not detected, low confidence, medium confidence, high confidence, ultra-high confidence). 71.6% of all possible scrambled disulfide connections were identified with high or ultra-high confidence, 17% of all possible scrambled disulfide connections were identified with medium confidence, and 11.3% of all possible scrambled disulfide connections were identified with low confidence.

FIGS. 14A, 14B and 14C depict various trypsin digestion protocols used to generate the peptide fragments comprising disulfide peptides and corresponding reduced partner peptides for mAb2. Three different protocols are indicated, including a standard operating procedure (SOP) for mAb2 (FIG. 14A), a SOP for mAb3 (FIG. 14B) and a low-pH digestion kit (FIG. 14C). Note that the SOP for mAb2 was used to generate the data depicted at FIG. 13.

FIGS. 15A and 15B depict alkylating agents iodo-acetamide (IAM) and N-ethyl maleimide (NEM) used with the procedures depicted at FIG. 14 to label free cysteine residues prior to trypsin digestion.

FIG. 16 depicts an overlay of UV chromatograms corresponding to trypsin-digested mAb2 using each of the digestion procedures illustratively depicted at FIGS. 14A-140, specifically the mAb2 SOP, the mAb3 SOP and the low-pH digestion kit. Samples run to obtain the chromatogram comprised 5 μg of trypsin digested mAb2 (or trypsin along with low pH resistant recombinant LysC in the case of the low pH digest protocol) in the presence of 2 mM glycine.

FIGS. 17A and 17B depict select time windows corresponding to the overlay of the UV chromatograms shown at FIG. 16. FIG. 17A depicts a time window from about 14 minutes to 30 minutes, and FIG. 17B depicts a time window from about 34 minutes to 49 minutes.

FIG. 18 depicts a graph representing MS signal for various native disulfides corresponding to mAb2, obtained by each of the three different digestion procedures illustratively depicted at FIGS. 14A-14C. Native disulfides include 0152H-0208H, C139H-C219L, C139H-C219L (missed, referring to miscleavage), C22H-C96H, C372H-C430H, C266H-C326H, C139L-C199L, 023L-093L, C231H-C231H and C234H3-C234H. For each disulfide, conditions from left to right include mAb2 SOP, mAb3 SOP and low-pH digestion kit procedure. The data depicted as MS signal includes all isotopic peaks of all major charge states. Samples run to generate the data at FIG. 18 include 5 μg of trypsin-digested mAb2 (or trypsin along with low pH resistant recombinant LysC in the case of the low pH digest protocol) in the presence of 2 mM glycine.

FIGS. 19A, 19B, 19C and 19D depict peak areas of peptides from various mAb domains (e.g., VH/VL, CH1, CL, CH2, CH3) of mAb2 digests. For each domain, peptides shown were generated via mAb2 SOP, mAb3 SOP, or the low pH digest kit, illustratively depicted at FIGS. 14A-140. FIG. 19A depicts the VH/VL domain, FIG. 19B depicts the CH1/CL domain, FIG. 19C depicts the CH2 domain, and FIG. 19D depicts the CH3 domain. FIG. 19A (top) includes peptide DYAMTWVR (SEQ ID NO: 17) and (bottom) includes peptide SGQSPQLLIYLGSNR (SEQ ID NO: 18). FIG. 19B (top) includes peptide DYFPEPVTVSWNSGALTSGVHTFPAVLQSSGLYSLSSVVTVPSSSLGTK (SEQ ID NO: 19) and (bottom) includes peptide ADYEK (SEQ ID NO: 20). FIG. 19C (top) includes peptide DTLMISR (SEQ ID NO: 39) and (bottom) includes VVSVLTVLHQDWLNGK (SEQ ID NO: 38). FIG. 19D (top) includes peptide GFYPSDIAVEWESNGQPENNYK (SEQ ID NO: 21) and (bottom) includes peptide TTPPVLDSDGSFFLYSR (SEQ ID NO: 22). For each peptide and corresponding digestion protocol depicted, sample conditions included 5 μg digested mAb2 and 2 mM glycine.

FIGS. 20A, 20B and 20C depict tables showing disulfide scrambling percentage for mAb2 digests as a function of various digestion protocols. Specifically, the mAb2 SOP, the mAb3 SOP and the low-pH digestion kit were individually tested for mAb2 to determine whether percent disulfide scrambling was dependent on digestion protocol. Shown are all possible disulfides and corresponding scrambling percentage depending on digestion protocol used. Data shown includes all isotopic peaks of all major charge states. For each digestion protocol depicted at FIGS. 20A-20C, sample conditions included 5 μg digested mAb2 and 2 mM glycine.

FIG. 20D depicts an equation for determining disulfide scrambling percentage, that is used for determining scrambling percentage in the experiments summarized at FIGS. 20A-20C.

FIG. 20E depicts a representative example of interference corresponding to the 0208H-093L disulfide peptide included in the table above at FIG. 20A.

FIGS. 21A and 21B depict relative abundance of high-abundance scrambled disulfides as determined via the quantitation shown at FIGS. 20A-20C. Specifically, FIG. 21A depicts C152H-C139H, corresponding to a disulfide of corresponding reduced partner peptides STSESTAALGCLVK (SEQ ID NO: 7) and GPSVFPLAPCSR (SEQ ID NO: 1). FIG. 21B depicts 023L-022H corresponding to a disulfide of corresponding reduced partner peptides DIVMTQSPLSLPVTPGEPASISCR (SEQ ID NO: 12) and LSCAGSGFTFR (SEQ ID NO: 8). For each of FIGS. 21A-21B, relative abundances are shown as a function of digestion protocol (mAb2 protocol, mAb3 protocol, or low-pH digestion protocol). As illustrated at both FIGS. 21A-21B, the chromatographs readily distinguish the high-abundance disulfides when digestion was performed with mAb2 protocol and mAb3 protocol, but the chromatograph of the scrambled disulfide is not distinguishable from baseline noise for the low-pH digestion condition. For each condition, samples included 5 μg of trypsin-digested mAb2 (or trypsin along with low pH resistant recombinant LysC in the case of the low pH digest protocol) and 2 mM glycine.

FIG. 22 depicts a UV overlay for a high-abundance scrambled disulfide (C152H-C139H) digested via each of the three different methodologies discussed above at FIG. 14, namely the mAb2 SOP, the mAb3 SOP and the low-pH digestion kit procedure. The UV chromatogram overlay illustrates that the C152H-C139H disulfide corresponding to reduced partner peptides STSESTAALGCLVK (SEQ ID NO: 7) and GPSVFPLAPCSR (SEQ ID NO: 1) is generated via the mAb2 and mAb3 digestion protocols, but not the low-pH digestion protocol. Sample conditions for all three digestion procedures included 5 μg of trypsin-digested mAb2 (or trypsin along with low pH resistant recombinant LysC in the case of the low pH digest protocol) and 2 mM glycine.

FIGS. 23A, 23B and 23C depict various trypsin digestion protocols used to generate fragments comprising disulfide peptides and corresponding reduced partner peptides for mAb 5. Three different protocols are indicated, including a mAb4 protocol (FIG. 23A), a mAb5 protocol (FIG. 23B) and a low-pH digestion kit (FIG. 23C).

FIG. 24 depicts an overlay of UV chromatograms corresponding to trypsin-digested mAb5 using each of the digestion procedures illustratively depicted at FIGS. 23A-23C, specifically the mAb4 protocol, the mAb5 protocol and the low-pH digestion kit. Samples run to obtain the chromatogram comprised 5 μg of trypsin digested mAb5 (or trypsin along with low pH resistant recombinant LysC in the case of the low pH digest protocol) in the presence of 2 mM glycine.

FIGS. 25A and 25B depict select time windows corresponding to the overlay of the UV chromatograms shown at FIG. 24. FIG. 25A depicts a time window from about 15 minutes to 33 minutes, and FIG. 25B depicts a time window from about 35 minutes to 67 minutes.

FIG. 26 depicts a graph representing MS signal for various native disulfides corresponding to mAb5, obtained by each of the three different digestion procedures illustratively depicted at FIGS. 23A-23C. Native disulfides include C22H-C96H, C145H-C201H, C221H-C213L, C221H-C213L (missed, referring to miscleavage), C227H-C227H, C230H-C230H, C262H-C322H, C368H-C426H, C23L-C88L, and C133L-C193L. For each disulfide, conditions from left to right include mAb4 protocol, mAb5 protocol and the low-pH digestion kit procedure. The data depicted as MS signal includes all isotopic peaks of all major charge states. Samples run to generate the data at FIG. 26 include 5 μg of trypsindigested mAb5 (or trypsin along with low pH resistant recombinant LysC in the case of the low pH digest protocol) in the presence of 2 mM glycine.

FIGS. 27A, 27B, 27C and 27D depict peak areas of peptides from various mAb domains (e.g., VH/VL, CH1, CL, CH2, CH3) of mAb5 digests. For each domain, peptides shown were generated via mAb4 protocol, mAb5 protocol, or the low pH digest kit, as illustratively depicted at FIGS. 23A-23C. FIG. 27A depicts the VH/VL domain, FIG. 27B depicts the CH1/CL domain, FIG. 27C depicts the CH2 domain and FIG. 27D depicts the CH3 domain. FIG. 27A (top) includes peptide EVQLVESGGGLVQPGGSLR (SEQ ID NO: 23) and (bottom) includes peptide DIQMTQSPSSLSASVGDR (SEQ ID NO: 24). FIG. 27B (top) includes peptide GPSVFPLAPSSK (SEQ ID NO: 25) and (bottom) ADYEK (SEQ ID NO: 40). FIG. 27C (top) includes peptide FNWYVDGVEVHNAK (SEQ ID NO: 26) and (bottom) ALPAPIEK (SEQ ID NO: 27). FIG. 27D (top) includes peptide DELTK (SEQ ID NO: 28) and (bottom) includes peptide TTPPVLDSDGSFFLYSK (SEQ ID NO: 29). For each peptide and corresponding digestion protocol depicted, sample conditions included 5 μg digested mAb2 and 2 mM glycine.

FIG. 28 depicts cysteine-containing peptides generated from trypsin digestion of mAb5. Shown for reference is cysteine residue number and indication of whether the cysteine is located on the heavy (H) or light (L) chain of mAb5. Tryptic peptides include LSCAASGFTSSSYAMNWVR (SEQ ID NO: 30), AEDTAVYYCAK (SEQ ID NO: 41), STSGGTAALGCLVK (SEQ ID NO: 31), DYFPEPVTVSWNSGALTSGVHTFPAVLQSSGLYSLSSVVTVPSSSLGTQTYICNVNHKPSNTK (SEQ ID NO: 32), SCDK (SEQ ID NO: 33), TPEVTCVVVDVSHEDPEVK (SEQ ID NO: 34), NQVSLTCLVK (SEQ ID NO: 42), WQQGNVFSCSVMHEALHNHYTQK (SEQ ID NO: 43), VTITCR (SEQ ID NO: 35), FSGSGSGTDFTLTISSLQPEDFATYYCQQSYSTLTFGQGTR (SEQ ID NO: 36), SGTASVVCLLNNFYPR (SEQ ID NO: 44), VYACEVTHQGLSSPVTK (SEQ ID NO: 45), and GEC/SFNRGEC. With regard to the cysteine C213L (GEC/SFNRGEC), GEC is the expected/predicted tryptic peptide, however SFNRGEC contains a trypsin missed cleavage which is quite common and abundant. When dealing with specific disulfide connections, both the GEC peptide and the SFNRGEC peptide contains the same cysteine residue, so the existence of one form confirms the existence of the other. Hence, the C213L residue is represented here as GEC/SFNRGEC, where SFNRGEC corresponds to SEQ ID NO: 16.

FIG. 29 illustrates all possible scrambled disulfide connections from mAb5, with the exception of hinge region peptide THTCPPCPAPELLGGPSVFLFPPKPK (SEQ ID NO: 47). Tandem mass spectrometry (MS/MS) using the targeted MS2 approach discussed herein was used to identify scrambled disulfide connections. 5 μg of trypsin-digested mAb5 was separated and partially reduced using 40 μM TCEP. 2 mM glycine was used to boost MS signal. The digestion procedure comprised the mAb4 protocol (see FIG. 23A). Each disulfide connection is coded according to confidence level in the determination (not detected, low confidence, medium confidence, high confidence, ultra-high confidence). 63.3% of all possible scrambled disulfide connections were identified with high or ultra-high confidence, 20% of all possible scrambled disulfide connections were identified with medium confidence, and 16.7% of all possible scrambled disulfide connections were identified with low confidence.

FIGS. 30A, 30B and 30C depict tables showing disulfide scrambling percentage for mAb5 digests as a function of various digestion protocols. Specifically, the mAb4 protocol, the mAb5 protocol and the low-pH digestion kit were individually tested for mAb5 to determine whether percent disulfide scrambling was dependent on digestion protocol. Shown are all possible disulfides and corresponding scrambling percentage depending on digestion protocol used. Data shown includes all isotopic peaks of all major charge states. The equation for determining disulfide scrambling percentage that was used for determining scrambling percentage in the experiments summarized at FIGS. 30A-30C is the equation depicted at FIG. 20D. For each digestion protocol depicted at FIGS. 30A-30C, sample conditions included 5 μg digested mAb5 and 2 mM glycine.

FIG. 31 depicts a set of m/z determinations corresponding to a dimer disulfide of STSGGTAALGCLVK (SEQ ID NO: 31). The top chromatograph illustrates m/z for the disulfide only, the middle chromatograph depicts m/z for just the fully reduced peptides corresponding to the disulfide peptide, and the bottom chromatograph depicts m/z for the partially-reduced peptides corresponding to the disulfide peptide. Together, the data indicates an altered isotope peak pattern in the partially-reduced sample. Isotope peaks are sharper in the top chromatograph due to the data (with no TCEP, hence “disulfide-only”) being collected with a higher MS1 resolution. MS1 resolution was decreased when performing post-column TCEP reduction to maximize the number of MS2 scans collected to increase signal near the apex of each reduced peptide peak using the targeted MS2 methodology.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims. Any embodiments or features of embodiments can be combined with one another, and such combinations are expressly encompassed within the scope of the present invention. In the following detailed description, reference is made to the accompanying drawings which form a part hereof, and in which are shown by way of illustration embodiments that may be practiced. It is to be understood that other embodiments may be utilized and structural or logical changes may be made without departing from the scope.

Various operations may be described as multiple discrete operations in turn, in a manner that may be helpful in understanding embodiments; however, the order of description should not be construed to imply that these operations are order-dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.)

Although any methods and materials similar or equivalent to those described herein can be used in the practice or testing of the present invention, the preferred methods and materials are now described. All patents, applications and non-patent publications mentioned in this specification are incorporated herein by reference in their entireties.

### Abbreviations Used Herein

ACN: Acetonitrile
AU: Absorbance Units
CH: Constant Heavy
CL: Constant Light
Cys: Cysteine
DDA: Data-Dependent Acquisition
EIC: Extracted Ion Chromatographs
E/S: Enzyme/Substrate
FA: Formic Acid
HILIC: Hydrophilic Interaction Liquid Chromatography
HPLC: High Performance Liquid Chromatography
IAM: iodo-acetamide
IgG: Immunoglobulin G
LC: Liquid Chromatography
LC-MS: Liquid Chromatography-Mass Spectrometry
mAb: Monoclonal Antibody
MPA: Mobile Phase A
MPB: Mobile Phase B
MS: Mass Spectrometry
MS/MS: Tandem Mass Spectrometry
MS1: First Mass Spectrometer of a Tandem Mass Spectrometer
MS2: Second Mass Spectrometer of a Tandem Mass Spectrometer
MW: Molecular Weight
NEM: N-ethyl maleimide
PRM: Parallel Reaction Monitoring
RPLC: Reversed Phase Liquid Chromatography
RPLC-MS/MS: Reversed Phase Liquid Chromatography Tandem Mass Spectrometry
TCEP: tris(2-carboxyethyl)phosphine
TFA: Trifluoroacetic Acid
TIC: Total Ion Current
UV: Ultraviolet
VH: Variable Heavy
VL: Variable Light

### Definitions

The term “antibody”, as used herein, is intended to refer to immunoglobulin molecules comprised of four polypeptide chains, two heavy (H) chains and two light (L) chains inter-connected by disulfide bonds (i.e., “full antibody molecules”), as well as multimers thereof (e.g. IgM) or antigen-binding fragments thereof. Each heavy chain is comprised of a heavy chain variable region (“HCVR” or “V_{H}”) and a heavy chain constant region (comprised of domains C_{H}1, C_{H}2 and C_{H}3). In various embodiments, the heavy chain may be an IgG isotype. In some cases, the heavy chain is selected from IgG1, IgG2, IgG3 or IgG4. In some embodiments, the heavy chain is of isotype IgG1 or IgG4, optionally including a chimeric hinge region of isotype IgG1/IgG2 or IgG4/IgG2. Each light chain is comprised of a light chain variable region (“LCVR or “V_{L}”) and a light chain constant region (C_{L}). The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The term “antibody” includes reference to both glycosylated and non-glycosylated immunoglobulins of any isotype or subclass. The term “antibody” includes antibody molecules prepared, expressed, created or isolated by recombinant means, such as antibodies isolated from a host cell transfected to express the antibody. For a review on antibody structure, see Lefranc et al., IMGT unique numbering for immunoglobulin and T cell receptor variable domains and Ig superfamily V-like domains, 27(1) Dev. Comp. Immunol. 55-77 (2003); and M. Potter, Structural correlates of immunoglobulin diversity, 2(1) Surv. Immunol. Res. 27-42 (1983).

The term antibody also encompasses “bispecific antibody”, which includes a heterotetrameric immunoglobulin that can bind to more than one different epitope. One half of the bispecific antibody, which includes a single heavy chain and a single light chain and six CDRs, binds to one antigen or epitope, and the other half of the antibody binds to a different antigen or epitope. In some cases, the bispecific antibody can bind the same antigen, but at different epitopes or non-overlapping epitopes. In some cases, both halves of the bispecific antibody have identical light chains while retaining dual specificity. Bispecific antibodies are described generally in U.S. Patent App. Pub. No. 2010/0331527 (Dec. 30, 2010).

The term “antigen-binding portion” of an antibody (or “antibody fragment”), refers to one or more fragments of an antibody that retain the ability to specifically bind to an antigen. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 241:544-546), which consists of a VH domain, (vi) an isolated CDR, and (vii) an scFv, which consists of the two domains of the Fv fragment, VL and VH, joined by a synthetic linker to form a single protein chain in which the VL and VH regions pair to form monovalent molecules. Other forms of single chain antibodies, such as diabodies are also encompassed under the term “antibody” (see e.g., Holliger et at. (1993) 90 PNAS U.S.A. 6444-6448; and Poljak et at. (1994) 2 Structure 1121-1123).

Moreover, antibodies and antigen-binding fragments thereof can be obtained using standard recombinant DNA techniques commonly known in the art (see Sambrook et al., 1989). Methods for generating human antibodies in transgenic mice are also known in the art. For example, using VELOCIMMUNE® technology (see, for example, U.S. Pat. No. 6,596,541, Regeneron Pharmaceuticals, VELOCIMMUNE®) or any other known method for generating monoclonal antibodies, high affinity chimeric antibodies to a desired antigen are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

The term “human antibody”, is intended to include antibodies having variable and constant regions derived from human germline immunoglobulin sequences. The human mAbs of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse), have been grafted onto human FR sequences. The term includes antibodies recombinantly produced in a non-human mammal, or in cells of a non-human mammal. The term is not intended to include antibodies isolated from or generated in a human subject.

The term “disulfide” as used herein is intended to refer to a covalent bond derived from two thiol groups. In proteins, for example monoclonal antibodies, these bonds from between the thiol groups of two cysteine amino acids. Disulfide bonds contribute to stabilizing protein globular structure and holding proteins in their respective conformation, thus having an important role in protein folding and stability. As discussed herein, a disulfide, or disulfide peptide, encompasses two peptides covalently bonded though cysteine residues on each corresponding peptide, and each of the two peptides in their reduced form are referred to as “reduced partner peptides.” Such disulfide peptides can be generated via protease digestion (e.g., trypsin protease digestion and/or recombinant Lys-C protease digestion) under non-reducing conditions, where disulfide bonds remain intact. Such disulfide peptides can then be reduced to their corresponding reduced partner peptides via a reducing agent (e.g., DTT, TCEP, etc.). As used herein, the term “scrambled disulfide” or “disulfide scrambling” encompasses disulfide bonds that are non-native to a particular biomolecule, such as a monoclonal antibody.

The term “hydrophilic interaction chromatography” or HILIC is intended to include a process employing a hydrophilic stationary phase and a hydrophobic organic mobile phase in which hydrophilic compounds are retained longer than hydrophobic compounds. In certain embodiments, the process utilizes a water-miscible solvent mobile phase.

The term “sample,” as used herein, refers to a mixture of molecules that comprises at least an analyte molecule, e.g., disulfide peptide and/or corresponding reduced partner peptides, such as obtained from a monoclonal antibody, that is subjected to manipulation in accordance with the methods of the invention, including, for example, separating, analyzing, extracting, or concentrating.

The terms “analysis” or “analyzing,” as used herein, are used interchangeably and refer to any of the various methods of separating, detecting, isolating, purifying, solubilizing, and/or characterizing molecules of interest (e.g., biomolecules including but not limited to monoclonal antibodies, disulfide peptides and/or corresponding reduced partner peptides). Examples include, but are not limited to, solid phase extraction, solid phase micro extraction, electrophoresis, mass spectrometry, e.g., ESI-MS, tandem mass spectrometry (MS/MS), or MALDI-MS, liquid chromatography e.g., high performance, e.g., reverse phase, normal phase, or size exclusion, ion-pair liquid chromatography, liquid-liquid extraction, e.g., accelerated fluid extraction, supercritical fluid extraction, microwave-assisted extraction, membrane extraction, soxhlet extraction, precipitation, clarification, electrochemical detection, staining, elemental analysis, Edmund degradation, nuclear magnetic resonance, infrared analysis, flow injection analysis, capillary electrochromatography, ultraviolet detection, and combinations thereof.

“Electrospray Ionization Mass Spectrometry” or “ESI-MS” is technique used in mass spectrometry to produce ions using an electrospray in which a high voltage is applied to a liquid to create an aerosol. For example, in electrospray, the ions are created from proteins/peptides in solution which allows fragile molecules to be ionized intact which may preserve non-covalent interactions. Electrospray ionization is the ion source of choice to couple liquid chromatography with mass spectrometry (LC-MS). The analysis can be performed online, by feeding the liquid eluting from the LC column directly to an electrospray, or offline, by collecting fractions to be later analyzed in a classical nanoelectrospray-mass spectrometry setup. LC-MS can be used to characterize proteins including quantifying biomarkers, analyzing sequence variants and identifying and quantifying disulfide peptides and corresponding reduced partner peptides.

“Tandem mass spectrometry” or “MS/MS” or “MS²” is a technique used for the analysis of biomolecules, such as proteins and peptides. In tandem mass spectrometry, a first mass analyzer (MS1) selects ions (e.g., samples ionized via ESI, MALDI, etc.) of one particular mass to charge ratio (m/z) (or range of mass to charge ratios) from ions supplied via an ion source. The ions are fragmented and a second mass analyzer (MS2) records the mass spectrum of the fragment ions. Tandem mass spectrometry involves three distinct steps of selection, fragmentation, and detection. The separation of these steps can be realized in space or in time. Typical tandem mass spectrometry in space instruments include QqQ (triple quadrupole), QTOF (quadrupole time-of-flight), hybrid ion trap/FTMS (Fourier transform mass spectrometry), etc. Tandem-in-time MS/MS instruments include ion trap and FT-ICR MS (Fourier-transform ion cyclotron resonance mass spectrometry). The step of fragmentation is generally done by colliding selected ions with a neutral gas in a process called collisional activation (CA) or collision-induced dissociation (CID).

“Partially reduced” or “partial reduction” as discussed herein encompasses treating a sample (e.g., biomolecule or fragments of a biomolecule) with a reductant at a concentration and/or for an amount of time so as to reduce some fraction of disulfide bonds present within the sample to their corresponding reduced counterparts, but where not all of the disulfides present in the sample are reduced.

“Parallel reaction monitoring” or “PRM” as discussed herein refers to a targeted proteomics technology used to quantify a plurality of proteins/peptides in the same experiment. In PRM, all fragment ions instead of only selected ones are measured after fragmentation of a selected precursor. PRM is typically performed on Orbitrap or Time of flight (ToF) analyser. PRM can reduce assay development time because no target transitions (product ions) need to be preselected. PRM eliminates most interferences, providing improved accuracy and attomole-level limits of detection and quantification.

“Data-dependent acquisition” or “DDA” as discussed herein refers to a mode of acquisition in tandem mass spectrometry. In DDA mode, the mass spectrometer selects the most intense peptide ions in a first stage of tandem mass spectrometry, and then they are fragmented and analyzed in a second stage of mass spectrometry.

General Description

Therapeutic antibodies are increasingly being used in the treatment of diseases such as cancer, infection, and other conditions. Therapeutic antibodies function, for example, by binding to an antigen (e.g., present on a target cell), thereby attracting disease-fighting molecules and/or triggering cell death via immune system responses and/or blocking a process (e.g., viral entry into a cell or interaction between receptor and ligand) that would otherwise lead to infection and/or disease. To be effective, a therapeutic antibody needs to be stable and properly folded. Improper folding and/or degraded stability can contribute to ineffectiveness of such molecules. One reason for improper folding and/or degraded stability includes the formation of non-native (e.g., scrambled) disulfide bonds at some point in the process of therapeutic antibody development. Thus, there is a need for methods that readily enable determination of scrambled disulfide bonding in therapeutic biomolecules, including but not limited to therapeutic antibodies.

Disclosed herein are new methodologies of mass spectrometry based characterization of scrambled disulfide bonds in biomolecules. The disclosed methodologies improve an ability to reliably detect and quantify scrambled disulfide bonds in biomolecules, including but not limited to monoclonal antibodies. Discussed herein, it was surprisingly found that improvement in mass spectral signal via the inclusion of a small molecule additive (e.g., glycine) in liquid chromatography mobile phase solutions was specifically dependent on concentration of reductant used to partially reduce sample components eluted from a liquid chromatography separation column. It was also surprisingly found that the improvement to mass spectral signal and the ability to use mass spectral analysis to detect reduced partner peptides corresponding to disulfide peptide fragments of biomolecules was highly dependent on selection of ion pairing agent included in the mobile phase during liquid chromatography separation. Still further, it was surprisingly found that biomolecule digestion conditions prior to the digested sample components being separated and analyzed via mass spectrometry could reliably induce artificial disulfide scrambling under certain conditions, and that this artificial disulfide scrambling could be avoided by performing the digestion procedures at an acidic pH. The above discoveries enable an improved ability to reliably detect and quantify scrambled disulfide bonds in biomolecules with high confidence using tandem mass spectrometry (MS/MS), as herein disclosed. Thus, the disclosed methodologies have a very broad range of applications in terms of enabling an ability to screen therapeutic biomolecules (e.g., monoclonal antibodies) for disulfide scrambling, which in turn may improve effectiveness of therapeutics derived from the use of such molecules.

Discussed herein, separation of protease-digested biomolecules via liquid chromatography methodology can comprise gradients of one or more buffers used. One or more of said buffers can in some examples include ion pairing agents, including but not limited to formate, acetate, TFA, and salts. In a preferred embodiment, the ion pairing agent comprises TFA.

In a case where two buffers are used, the concentration of a first buffer can decrease while the concentration or percentage of the second buffer increases over the course of the chromatography run. For example, the percentage of the first buffer can decrease from about 100%, about 99%, about 95%, about 90%, about 85%, about 80%, about 75%, about 70%, about 65%, about 60%, about 50%, about 45%, or about 40% to about 0%, about 1%, about 5%, about 10%, about 15%, about 20%, about 25%, about 30%, about 35%, or about 40% over the course of the chromatography run. As another example, the percentage of the second buffer can increase from about 0%, about 1%, about 5%, about 10%, about 15%, about 20%, about 25%, about 30%, about 35%, or about 40% to about 100%, about 99%, about 95%, about 90%, about 85%, about 80%, about 75%, about 70%, about 65%, about 60%, about 50%, about 45%, or about 40% over the course of the same run. Optionally, the concentration or percentage of the first and second buffer can return to their starting values at the end of the chromatography run. As an example, the percentage of the first buffer can change in five steps from 85% to 63% to 59% to 10% to 85%; while the percentage of the second buffer in the same steps changes from 15% to 37% to 41% to 90% to 15%. The percentages can change gradually as a linear gradient or in a non-linear (e.g., stepwise) fashion. For example, the gradient can be multiphasic, e.g., biphasic, triphasic, etc. In some embodiments, the methods described herein use a decreasing acetonitrile buffer gradient which corresponds to increasing polarity of the mobile phase without the use of ion pairing agents.

In some embodiments, applying a mobile gradient to the separation column includes applying a first mobile gradient buffer to the separation column, wherein the first mobile phase buffer includes TFA and a small molecule additive (e.g., an amino acid) and applying a second mobile gradient to the separation column, wherein the second mobile phase buffer comprises TFA in ACN and a small molecule additive (e.g., an amino acid).

In various embodiments, the small molecule additive is selected from glycine, alanine, serine, valine, N-acetyl glycine, methionine, β-alanine, aspartic acid, or N-methyl glycine. In some cases, the amino acid is selected from glycine, alanine, serine or valine. In some embodiments, the amino acid is alanine. In some embodiments, the amino acid is serine. In some embodiments, the amino acid is valine. In some embodiments, the amino acid in the first mobile phase buffer is glycine. In some embodiments, the amino acid in the second mobile phase buffer is glycine. In some embodiments, the amino acid in the first and second mobile phase buffers is glycine. In some embodiments, the small molecule additive (e.g., the amino acid) in the first and/or second mobile phase buffer is one of the small molecules (e.g., modified amino acids) or other amino acids identified above or herein.

The concentration of the small molecule additive (e.g., amino acid) in the mobile phase buffer is about 0.5 mM to about 5 mM, such as between about 0.5 mM to about 3 mM, about 1 mM and about 2 mM, including 0.5 mM, 0.6 mM, 0.7 mM, 0.8 mM, 0.9 mM, 1.0 mM, 1.1 mM, 1.2 mM, 1.3 mM, 1.4 mM, 1.5 mM, 1.6 mM, 1.7 mM, 1.8 mM, 1.9 mM, 2.0 mM, 2.1 mM, 2.2 mM, 2.3 mM, 2.4 mM, 2.5 mM, 2.6 mM, 2.7 mM, 2.8 mM, 2.9 mM, 3.0 mM, 3.1 mM, 3.2 mM, 3.3 mM, 3.4 mM, 3.5 mM, 3.6 mM, 3.7 mM, 3.8 mM, 3.9 mM, 4.0 mM, 4.1 mM, 4.2 mM, 4.3 mM, 4.4 mM, 4.5 mM, 4.6 mM, 4.7 mM, 4.8 mM, 4.9 mM, or 5.0 mM. In some embodiments, the small molecule additive (e.g., amino acid) is less than 5 mM. In some embodiments, the small molecule additive is glycine at a concentration of less than 5 mM. In some embodiments, the amino acid in the first mobile phase buffer is glycine and the concentration is between about 1 to about 2 mM glycine. In some embodiments, the amino acid in the second mobile phase buffer is glycine and the concentration is between about 1 to about 2 mM glycine. In some embodiments, the glycine concentration in the first mobile phase buffer is about 1 mM. In some embodiments, the glycine concentration in the first mobile phase buffer is about 2 mM. In some embodiments, the amino acid in the second mobile phase buffer is glycine and the concentration is between about 1 to about 2 mM glycine. In some embodiments, the glycine concentration in the second mobile phase buffer is about 1 mM. In some embodiments, the glycine concentration in the second mobile phase buffer is about 2 mM. In some embodiments, the amino acid in the first and second mobile phase buffer is glycine and the concentration is between about 1 to about 2 mM glycine.

In some embodiments, the TFA concentration in the first mobile phase is about 0.03% to 0.15% TFA in H₂O, such as about 0.03% to 0.1%. In some embodiments, the TFA concentration is about 0.05% to about 0.1% TFA in H₂O. For example, the TFA concentration is about 0.03%, 0.04%, 0.05%, 0.06%, 0.07%, 0.08%, 0.09%, or 0.1% in H₂O. In some embodiments, the TFA concentration in the second mobile phase comprises about 0.05% TFA in 80% ACN and 20% H₂O or about 0.1% TFA in 80% ACN and 20% H₂O. In some embodiments, the concentration of ACN in the second mobile phase is about 60% to 100%, such as between 80% and 100%, including 60%, 65%, 70%, 75%, 80%, 85%, 90%, 95% or 100%.

In some embodiments, the sample comprises peptides. In some embodiments, the sample comprises peptides linked via cysteine residues through a disulfide bond linkage. For example, the sample can include disulfide peptides obtained via proteolytic digestion of a monoclonal antibody, or other biomolecule, under non-reducing conditions. In some embodiments, the monoclonal antibody is of isotype IgG1, IgG2, IgG3, IgG4, or mixed isotype.

In some embodiments, the method includes preparing the sample prior to contacting the sample to a separation column under conditions that permit sample components to bind to the substrate. In some embodiments, sample preparation includes contacting the sample with a denaturation/alkylation solution under conditions that permit sample denaturation and alkylation. In examples, a denaturant is urea. A concentration of urea sufficient to cause sample denaturation (e.g., protein denaturation) may be between 7-9M urea, for example 8 M urea. In examples, the denaturation/alkylation solution includes an alkylating agent. The alkylating agent may comprise iodo-acetamide (IAM) in an example. Additionally or alternatively, the alkylating agent may comprise N-ethyl maleimide (NEM). In examples, the alkylating agent in the denaturation/alkylation solution is at a concentration between about 0.5 mM to about 10 mM, for example about 1 mM to about 8 mM. In some examples, the alkylating agent in the denaturation/alkylation solution is NEM and the concentration is about 6 mM to about 10 mM, for example 8 mM. In some examples, the alkylating agent in the denaturation/alkylation solution is IAM and the concentration is about 0.5 mM to about 5 mM, for example about 1 mM, or about 2 mM, or about 3 mM or about 4 mM or about 5 mM, for example 2.4 mM. In some examples, the denaturation/alkylation solution is contacted to the sample for a time period of about 10 minutes to about 60 minutes, for example 20 minutes, or 30 minutes, or 40 minutes, or 50 minutes. In examples the denaturation/alkylation solution is of a temperature between about 40° C. and about 60° C., for example about 50° C. In examples, a pH of the denaturation/alkylation solution is acidic. For example, the pH may be between about 5 and about 6.5, for example about 5.5 and about 6.0, for example about 5.7.

In some examples, preparing the sample prior to contacting the sample to a separation column under conditions that permit sample components to bind to the substrate include, subsequent to contacting the sample with the denaturation/alkylation solution, contacting the sample with a pre-digestion solution. In examples, the pre-digestion solution comprises a protease, for example a serine protease. In examples, the protease is endoproteinase LysC. In examples, the protease is a recombinant protease, for example recombinant endoproteinase LysC. In examples, the pre-digestion solution includes the protease at an enzyme/substrate ratio of about 1:2 to about 1:20, for example about 1:5 to about 1:15, for example about 1:10, respectively. In examples, the pre-digestion solution is contacted to the sample for about 30 minutes to 2 hours, for example 1 hour. In examples, the pre-digestion solution is of a temperature of about 35-40° C., for example about 37° C. In examples, the pre-digestion solution is of an acidic pH, for example a pH of between about 5 to about 6, for example about 5.2-5.5, for example about 5.3.

In some examples, preparing the sample prior to contacting the sample to a separation column under conditions that permit sample components to bind to the substrate includes, subsequent to contacting the sample with the denaturation/alkylation solution and contacting the sample with a pre-digestion solution, contacting the sample with a digestion solution. In examples, the digestion solution comprises a first protease, for example a serine protease. In examples, the first protease is endoproteinase LysC. In examples, the first protease is a recombinant protease, for example recombinant endoproteinase LysC. In examples, the digestion solution includes the first protease at an enzyme/substrate ratio of about 1:2 to about 1:20, for example about 1:5 to about 1:15, for example about 1:10, respectively. In examples, the digestion solution comprises a second protease, for example a serine protease. In examples, the second protease is trypsin. In examples, the digestion solution includes the second protease at an enzyme/substrate ratio of about 1:2 to about 1:10, for example about 1:5, respectively. In examples, the digestion solution is contacted to the sample for about 1 hour to about 4 hours, for example 3 hours. In examples, the digestion solution is of a temperature of about 35-40° C., for example about 37° C. In examples, the digestion solution is of an acidic pH, for example a pH of between about 5 to about 6, for example about 5.2-5.5, for example about 5.3.

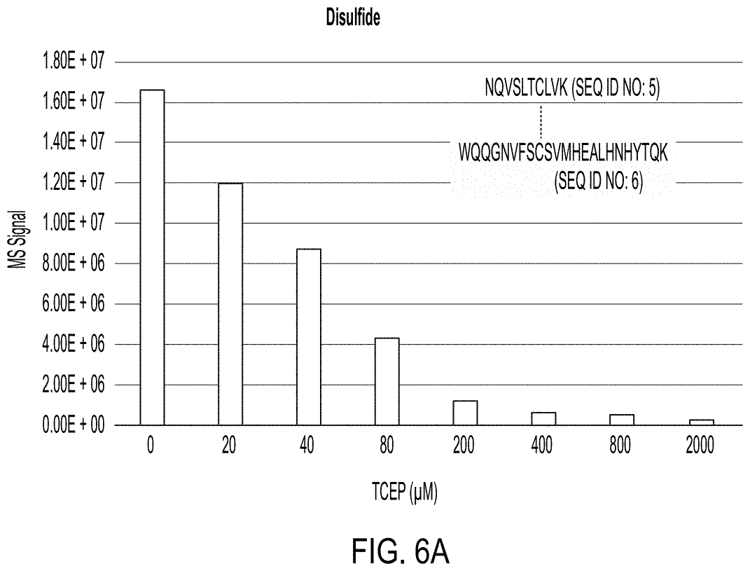

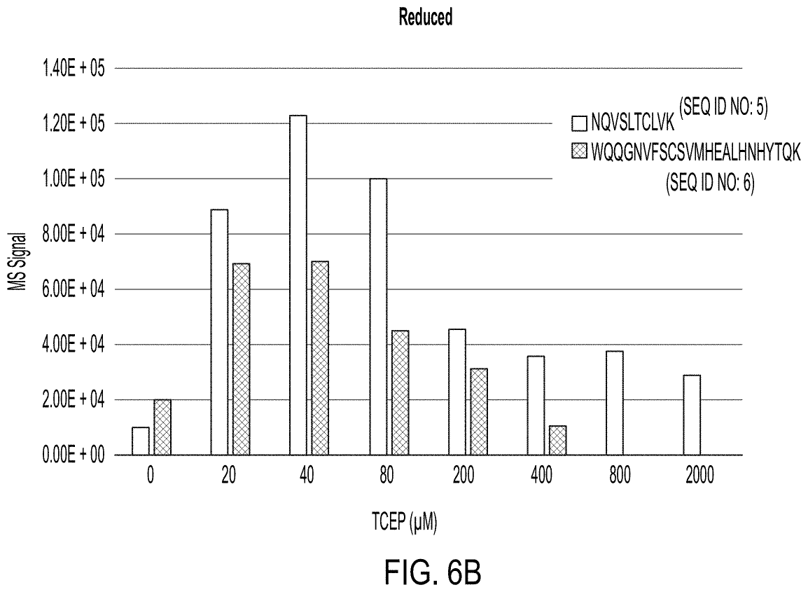

Discussed herein, a partial reduction procedure is performed on sample components eluted from the liquid chromatography column following their separation. It may be understood that, prior to the partial reduction procedure, sample components comprise non-reduced digested biomolecule(s) (e.g., monoclonal antibody). The partial reduction procedure includes treating eluted sample components with a reductant. In examples, the reductant is TCEP. In examples, a concentration of the reductant used to partially reduce eluted sample components is about 20 μM to about 100 μM, for example about 30 μM to about 60 μM, for example about 40 μM. As discussed below in Example 1, and at least FIGS. 6A-6B, it was surprisingly found that MS signal of reduced partner peptides was highly dependent on a specific range of TCEP concentration (about 20 μM to about 100 μM), with decreased MS signal being associated with TCEP concentrations outside of (e.g., both higher and lower concentrations) the specific range. This finding was particularly surprising as it was expected that greater concentrations of TCEP (e.g., 400 μM to 2 mM and higher) would result in increased abundance of reduced partner peptides corresponding to disulfide peptides, thereby leading to increased MS signal for the corresponding reduced partner peptides at higher TCEP concentrations. The finding that lower TCEP concentrations (e.g., 20-100 μM, for example about 40 μM) improved, rather than degraded, the ability to detect corresponding reduced partner peptides, was thus unexpected. In examples, the partial reduction procedure includes additionally treating eluted sample components with NH₄OH. In examples, a final percentage of NH₄OH is about 0.05% to about 0.2%, for example about 0.12%. In examples, the partial reduction procedure is performed on eluted sample components for a duration of about 0.5 seconds to about 5 seconds, for example about 1-3 seconds, for example about 2 seconds. In examples, an efficiency corresponding to the partial reduction procedure is about 1-3%.

In some embodiments, the separation column is a liquid chromatography (LC) separation column. Liquid chromatography, including HPLC, can be used to separate structures, such as peptides, including disulfide peptides. Various forms of liquid chromatography can be used to separate these structures, including anion-exchange chromatography, reversed-phase HPLC, size-exclusion chromatography, high-performance anion-exchange chromatography, and normal phase (NP) chromatography, including NP-HPLC (see, e.g., Alpert et al., J. Chromatogr. A 676:191-202 (1994)). Hydrophilic interaction chromatography (HILIC) is a variant of NP-HPLC that can be performed with partially aqueous mobile phases, permitting normal-phase separation of peptides, disulfide peptides, carbohydrates, nucleic acids, and many proteins. The elution order for HILIC is least polar to most polar, the opposite of that in reversed-phase HPLC. HPLC can be performed, e.g., on an HPLC system from Waters (e.g., Waters 2695 Alliance HPLC system), Agilent, Perkin Elmer, Gilson, etc.

NP-HPLC, preferably HILIC, can in some examples be used in the methods described herein. NP-HPLC separates analytes based on polar interactions between the analytes and the stationary phase (e.g., substrate). The polar analyte associates with and is retained by the polar stationary phase. Adsorption strengths increase with increase in analyte polarity, and the interaction between the polar analyte and the polar stationary phase (relative to the mobile phase) increases the elution time. Use of more polar solvents in the mobile phase will decrease the retention time of the analytes while more hydrophobic solvents tend to increase retention times.

Various types of substrates can be used with NP-HPLC, e.g., for column chromatography, including silica, amino, amide, cellulose, cyclodextrin and polystyrene substrates. Examples of useful substrates, e.g., that can be used in column chromatography, include: polySulfoethyl Aspartamide (e.g., from PolyLC), a sulfobetaine substrate, e.g., ZIC®-HILIC (e.g., from SeQuant), POROS® HS (e.g., from Applied Biosystems), POROS® S (e.g., from Applied Biosystems), PolyHydroethyl Aspartamide (e.g., from PolyLC), Zorbax 300 SCX (e.g., from Agilent), PolyGLYCOPLEX® (e.g., from PolyLC), Amide-80 (e.g., from Tosohaas), TSK GEL® Amide-80 (e.g., from Tosohaas), Polyhydroxyethyl A (e.g., from PolyLC), Glyco-Sep-N (e.g., from Oxford GlycoSciences), and Atlantis HILIC (e.g., from Waters). In some embodiments, the disclosed methods include columns that utilize one or more of the following functional groups: carbamoyl groups, sulfopropyl groups, sulfoethyl groups (e.g., poly (2-sulfoethyl aspartamide)), hydroxyethyl groups (e.g., poly (2-hydroxyethyl aspartamide)) and aromatic sulfonic acid groups.

In some embodiments, reversed phase HPLC can be used with the methods described herein. Reversed phase HPLC separates analytes based on nonpolar interactions between analytes and the stationary phase (e.g., substrate). The nonpolar analyte associates with and is retained by the nonpolar stationary phase. Adsorption strengths increase with analyte nonpolarity, and the interaction between the nonpolar analyte and the nonpolar stationary phase (relative to the mobile phase) increases the elution time. Use of more nonpolar solvents in the mobile phase will decrease the retention time of the analytes, while more polar solvents tend to increase retention times.

The column temperature can be maintained at a constant temperature throughout the chromatography run, e.g., using a commercial column heater. In some embodiments, the column is maintained at a temperature between about 18° C. to about 70° C., e.g., about 30° C. to about 60° C., about 40° C. to about 50° C., e.g., at about 20° C., about 25° C., about 30° C., about 35° C., about 40° C., about 45° C., about 50° C., about 55° C., about 60° C., about 65° C., or about 70° C. In some embodiments, the column temperature is about 40° C.

The flow rate of the mobile phase can be between about 0 to about 100 ml/min. For analytical proposes, flow rates typically range from 0 to 10 ml/min, for preparative HPLC, flow rates in excess of 100 ml/min can be used. For example, the flow rate can be about 0.5, about 1, about 1.5, about 2, about 2.5, about 3, about 3.5, about 4, about 4.5, or about 5 ml/min (or higher for preparative HPLC). Substituting a column having the same packing, the same length, but a smaller diameter requires a reduction in the flow rate in order to retain the same retention time and resolution for peaks as seen with a column of wider diameter. In some embodiments, a flow rate equivalent to about 1 ml/min in a 4.6×100 mm, 5 μm column is used.

In some embodiments, the run time can be between about 15 to about 240 minutes, e.g., about 20 to about 70 min, about 30 to about 60 min, about 40 to about 90 min, about 50 min to about 100 min, about 60 to about 120 min, about 50 to about 80 min.

In examples, following the partial reduction procedure, the partially reduced sample is analyzed via mass spectrometry. In examples, the partially reduced sample includes disulfide peptides and corresponding reduced partner peptides. In examples, the disulfide peptides and corresponding reduced partner peptides enter the mass spectrometer at about a same time.

In examples, analyzing the sample via mass spectrometry includes, via reliance on a tandem mass spectrometer configuration, obtaining a MS1 spectra and a MS2 spectra. In examples, obtaining the MS2 spectra involves a targeted MS2 approach. Such a targeted MS2 approach can include targeting just the protease-digested (e.g., trypsin-digested) reduced partner peptides corresponding to disulfide peptides that contain a cysteine residue. In examples, targeting just the corresponding reduced partner peptides that contain cysteine enables dramatically simpler characterization of any scrambled disulfides, as compared to an approach where MS/MS is used in an attempt to target disulfide peptides, due to disulfide peptides possessing exponentially more possible fragmentations compared to their corresponding reduced partner peptides. Using a monoclonal antibody as an example, such a monoclonal antibody may contain a particular number of cysteine residues (e.g. 16). By employing the partial reduction procedure discussed herein, just 15 reduced tryptic peptides (15 tryptic peptides minus the hinge region of the antibody) that contain cysteine need be targeted at the MS2 stage. Thus, in examples, analyzing the sample via mass spectrometry can include building a PRM inclusion list with just the protease-generated fragments that contain cysteine, and scanning across an entire gradient. In examples, MS1 resolution can be lowered for an increased number of MS2 scans.

In examples, analyzing the sample via mass spectrometry includes assigning a disulfide identification confidence score for each possible disulfide peptide possibility in a particular biomolecule. Assigning the disulfide identification confidence score, in examples, includes assigning a point for each query answered in the affirmative out of a number of queries pertaining to the generated MS/MS data. In examples, the queries can include but are not limited to whether an MS1 mass of the disulfide peptide is identified, whether an MS1 mass of a first corresponding reduced peptide is identified, whether an MS1 mass of a second corresponding reduced peptide is identified, whether a byonic MS2 identification of the first corresponding reduced peptide is identified with a score greater than a predetermined threshold, and whether a byonic MS2 identification of the second corresponding reduced peptide is identified with a score greater than another predetermined threshold. In examples, the predetermined thresholds are the same, although it is within the scope of this disclosure that the predetermined thresholds be different. In examples, analyzing the sample via mass spectrometry includes assigning a scrambling percentage for each possible disulfide connection in a particular biomolecule (e.g., monoclonal antibody).

### EXAMPLES

The following examples are put forth so as to provide those of ordinary skill in the art with a complete disclosure and description of how to make and use the methods of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Optimized Post-Column Partial Reduction of Disulfides and MS Signal Boosting

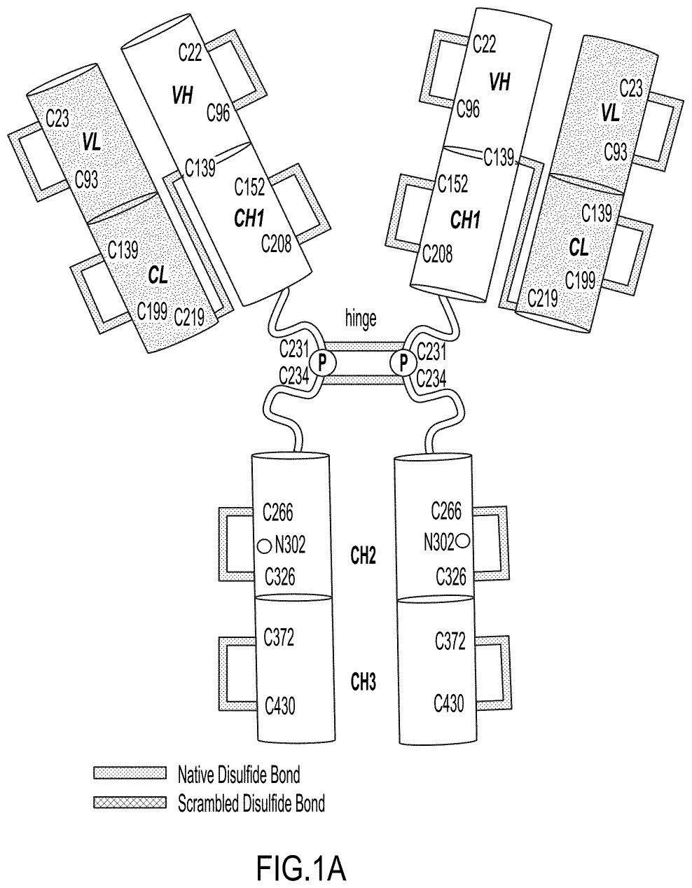

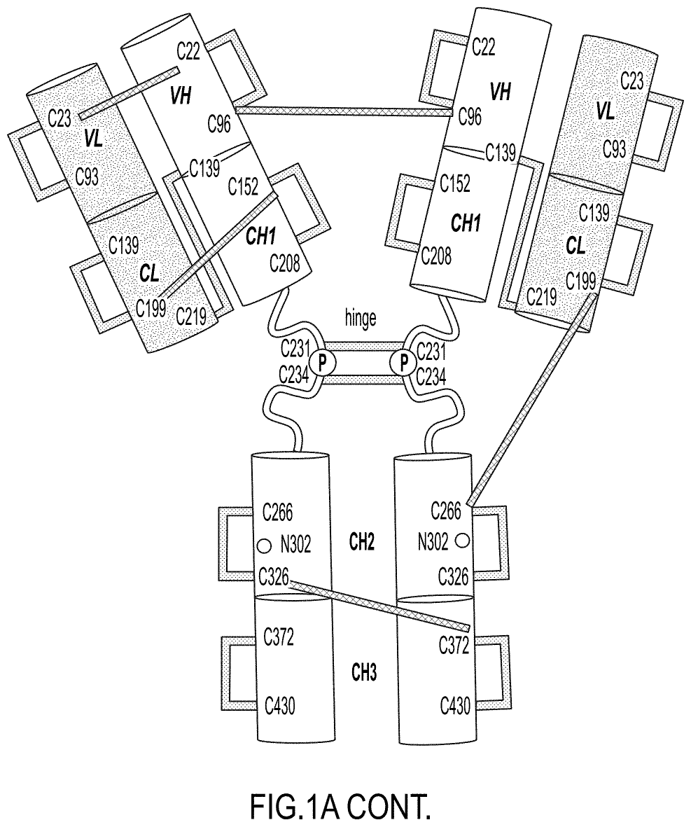

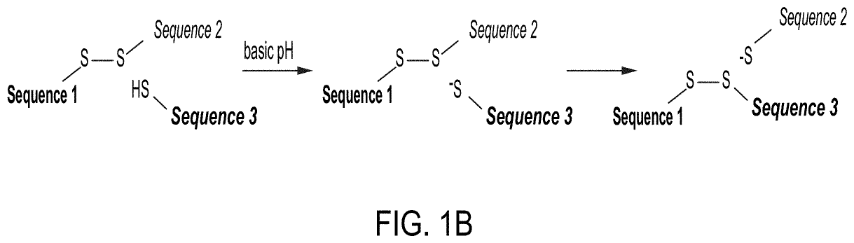

Antibodies (e.g., monoclonal antibodies) and other biomolecules harbor native disulfide bonds that contribute to stability and effective functionality. However, under certain conditions (e.g., alkaline conditions), non-native (also referred to herein as scrambled) disulfide bonding can occur, thereby degrading stability and functional effectiveness. FIG. 1A depicts a mAb with a plurality of native disulfide bonds (left), and the same mAb with a number of scrambled disulfide bonds (right). FIG. 1B illustrates a simplified scheme illustrating how disulfide bonding can rearrange (e.g., scramble) under basic conditions. This disclosure pertains to detecting scrambled disulfide bonds in biomolecules, with an emphasis on mAbs.

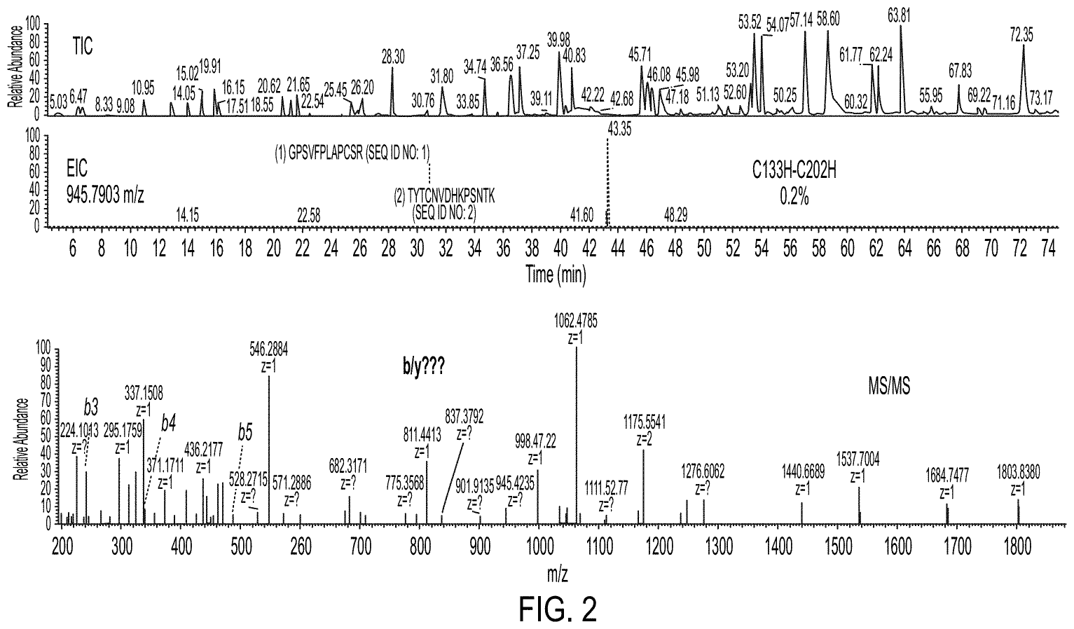

Therapeutic mAbs (or other therapeutic biomolecules) with scrambled disulfides could exhibit degraded functionality, hence an ability to detect scrambled disulfides is of importance. FIG. 2 illustrates that it can be challenging to unequivocally identify low-abundance scrambled disulfides in mAbs. Shown at FIG. 2 is a disulfide (C133H-C202H, relative abundance of 0.2%) formed from corresponding reduced partner peptides GPSVFPLAPCSR (SEQ ID NO: 1) and TYTCNVDHKPSNTK (SEQ ID NO: 2) as analyzed via liquid chromatography mass spectrometry (LC-MS) (top panels), and tandem mass spectrometry (MS/MS) analysis (bottom panel). The EIC illustrates the m/z ratio corresponding to the disulfide, but the MS/MS analysis is too complex to identify fragments corresponding to TYTCNVDHKPSNTK (SEQ ID NO: 2).

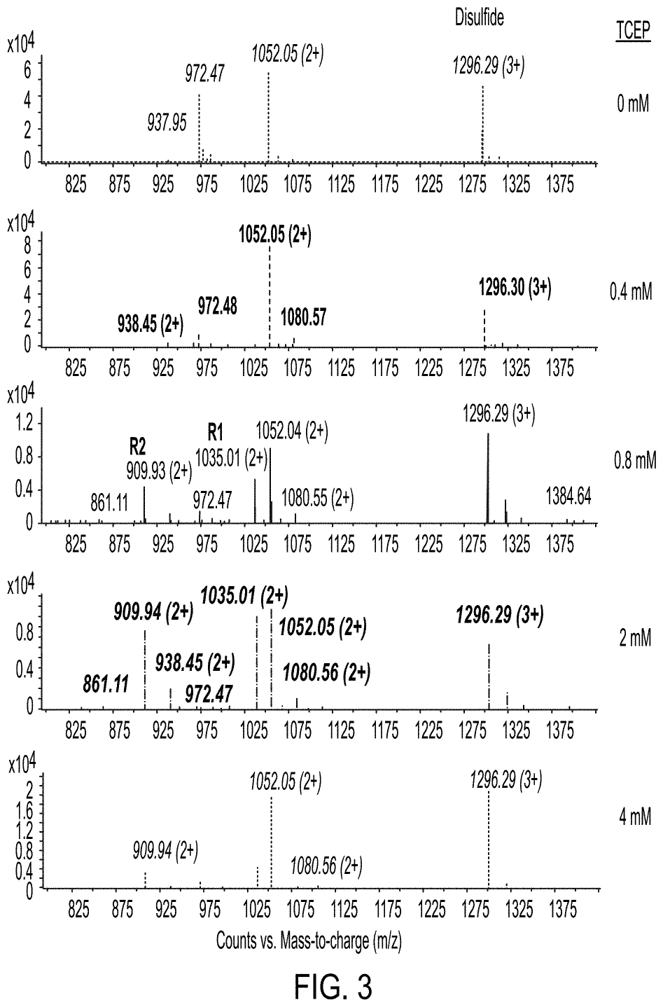

The issue of complexity associated with fragmenting a disulfide pair in a MS/MS analysis could be reduced by at least partially reducing the disulfide pair prior to mass spectral analysis. Turning to FIG. 3, depicted is a series of plots depicting counts vs. mass-to-charge (m/z) of a disulfide and correspondingly reduced partner peptides as a function of increasing TCEP concentration (0 mM, 0.4 mM, 0.8 mM, 2 mM, 4 mM). Briefly, a mAb was digested with trypsin under non-reducing conditions to maintain disulfide connections, and the non-reduced digest was separated via HPLC. Following separation, TCEP at the illustrated concentrations was reacted with the eluted disulfides for 1-3 seconds (e.g., partial reduction) before entering the mass spectrometer. Furthermore, NH₄OH (final concentration 0.12%) was added along with the TCEP to increase effectiveness of TCEP at reducing disulfides. Using this methodology the disulfide co-elutes with the reduced peptides such that all components have exactly the same retention time. For the experimental procedure, the digested mAb was run through the column at 50 μL/min (0.02% TFA, 0.08% FA), and the TCEP and NH₄OH was added to the sample post-separation via a syringe and mixing tee. The experiments illustrate highest signal for the reduced partner peptides at 2 mM TCEP.

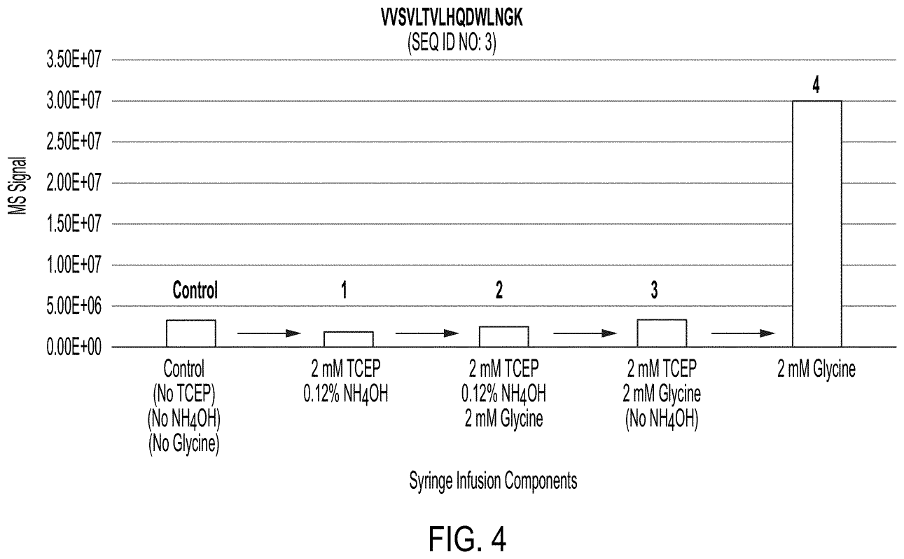

Because low-abundance disulfides may be difficult to detect, identify, and quantify if MS signal is too low, experiments were performed to determine whether addition of glycine to the mobile phase eluent containing TCEP could improve MS signal of scrambled disulfides. Specifically, an experiment was performed to determine whether glycine could improve MS signal in samples that also include 2 mM TCEP. The experimental procedure included examining MS signal of a peptide fragment VVSVLTVLHQDWLNGK (SEQ ID NO: 3) from mAb1, under various conditions as depicted at FIG. 4. The results indicate that 2 mM TCEP suppresses a MS signal boost otherwise observed by the addition of glycine (2 mM). Proceeding from left to right, sample 1 containing TCEP and NH₄OH exhibited a decreased signal as compared to control lacking TCEP, NH₄OH and glycine. Addition of 2 mM glycine to a sample containing TCEP and NH₄OH (sample 2) showed only marginal improvement, and removal of NH₄OH while maintaining addition of TCEP and glycine (sample 3) showed only marginal improvement in MS signal over sample 3. Only sample 4 (2 mM glycine in absence of TCEP and NH₄OH) showed a substantial MS signal boost, indicating that TCEP at 2 mM suppresses any MS signal boosting effect of 2 mM glycine (compare sample 3 to sample 4).

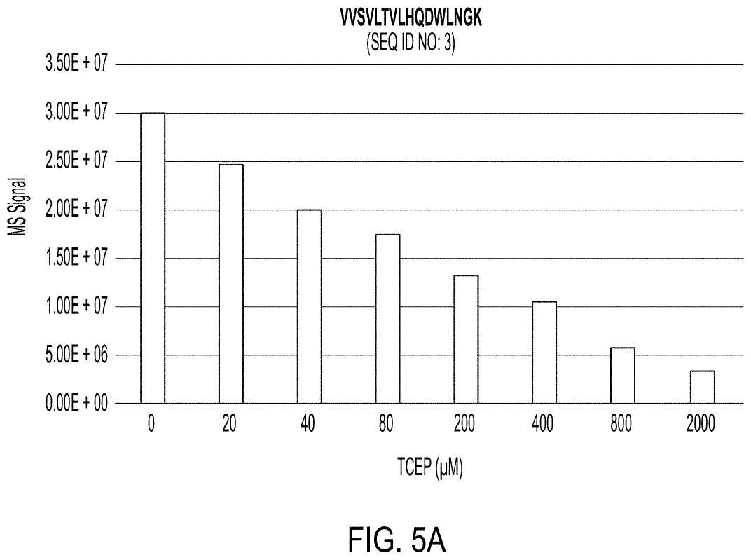

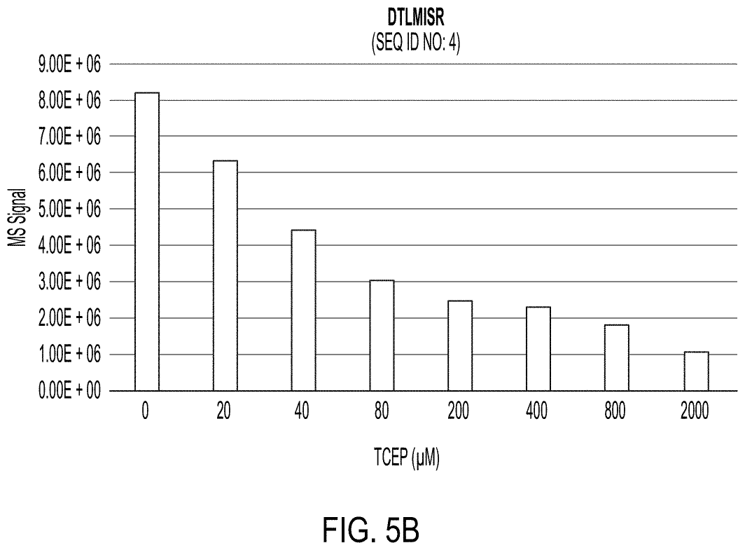

This suppressive effect of TCEP on glycine-induced MS signal boost was further examined in dose-response studies. FIG. 5A illustrates MS signal of a peptide fragment VVSVLTVLHQDWLNGK (SEQ ID NO: 3) from mAb1 across a range of TCEP concentrations from 0 μM to 2000 μM, and FIG. 5B illustrates MS signal of another peptide DTLMISR (SEQ ID NO: 4) across the same range of TCEP concentrations. Both FIG. 5A and FIG. 5B illustrate that MS signal decreases with increasing concentrations of TCEP. For each of FIGS. 5A-5B, sample conditions included mAb1 at a concentration of 0.1 μg, 0.05% TFA and 2 mM glycine, with the indicated concentrations of TCEP.

The results of FIGS. 5A-5B illustrated a higher MS signal associated with lower TCEP concentrations. Further experiments were conducted to determine whether lower concentrations of TCEP could at least partially avoid the suppressive effect on MS signal, while retaining an ability to detect reduced partner peptides corresponding to a disulfide-linked peptide pair. Depicted at FIG. 6A is MS signal for a disulfide corresponding to peptides NQVSLTCLVK (SEQ ID NO: 5) and WQQGNVFSCSVMHEALHNHYTQK (SEQ ID NO: 6), and depicted at FIG. 6B is MS signal for the corresponding reduced partner peptides. For each of FIGS. 6A-6B, varying TCEP concentrations were tested, ranging from 0 μM to 2000 μM, similar to that discussed above for FIGS. 5A-5B. Sample conditions at FIGS. 6A-6B included trypsin digested mAb1 at a loading amount of 1 μg, 0.05% TFA and 2 mM glycine, and with the specified TCEP concentrations. The data indicates that increasing concentrations of TCEP suppress MS signal corresponding to the disulfide (FIG. 6A), and that there is a range (about 20 μM to about 100 μM) where TCEP effectively reduces a disulfide-linked peptide pair and also where MS signal is not undesirably suppressed. The increased MS signal for corresponding reduced partner peptides (FIG. 6B) is desirable for MS/MS identification. The best MS signal was seen to be at about 40 μM TCEP.

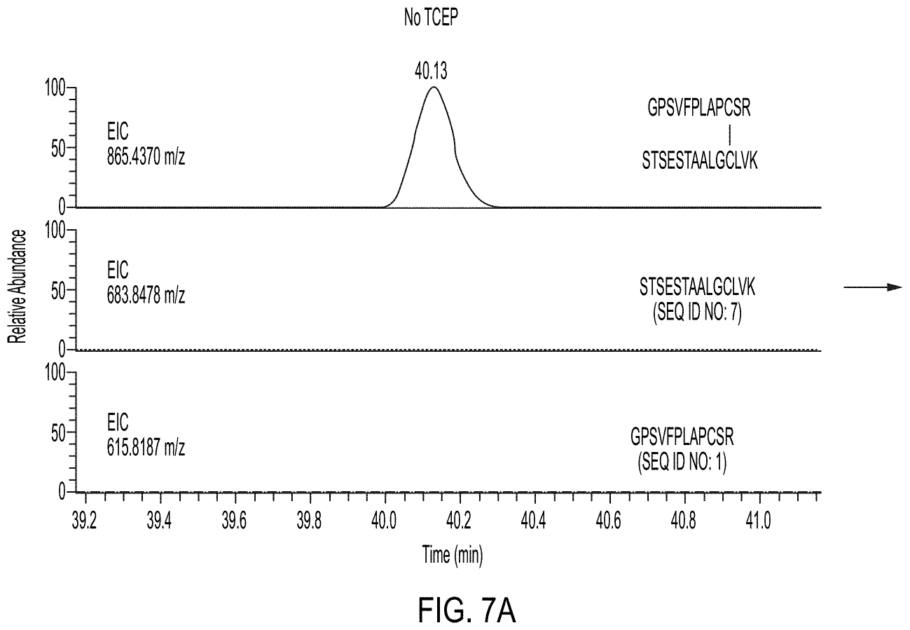

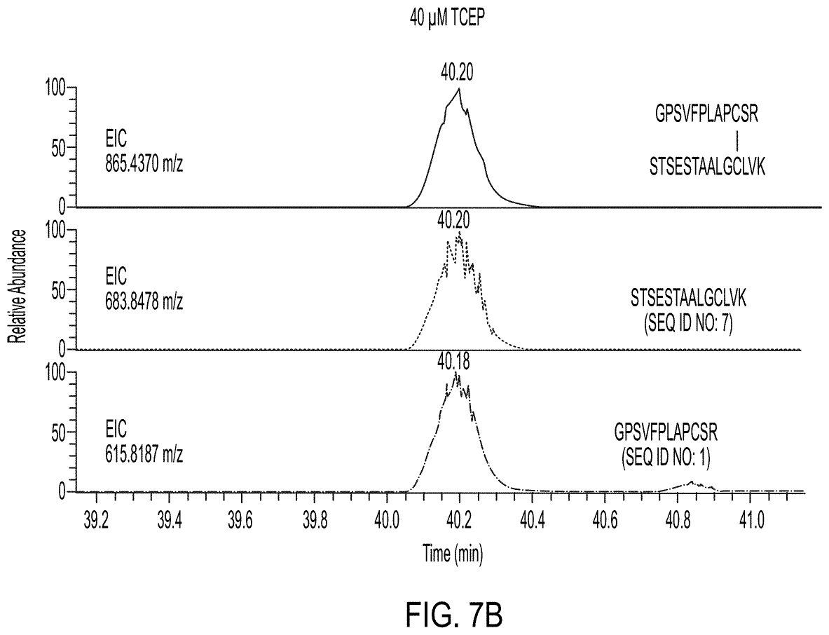

Accordingly, further experiments were performed with 40 μM TCEP for the partial reduction of disulfides into corresponding reduced partner peptides. Turning to FIGS. 7A-7B, depicted are relative abundances for a disulfide comprised of GPSVFPLAPCSR (SEQ ID NO: 1) and STSESTAALGCLVK (SEQ ID NO: 7) and the corresponding reduced partner peptides, without treatment with TCEP (FIG. 7A) and with partial reduction of the disulfide via treatment with 40 μM TCEP (FIG. 7B). The peptide fragments analyzed were generated from trypsin digestion of mAb2 prior to being subjected to H PLC. In an absence of partial reduction of the disulfide, no signal was seen for the corresponding reduced partner peptides (FIG. 7A), while good signal was observed for the corresponding reduced partner peptides when the disulfide peptide is partially reduced by 40 μM TCEP (FIG. 7B).

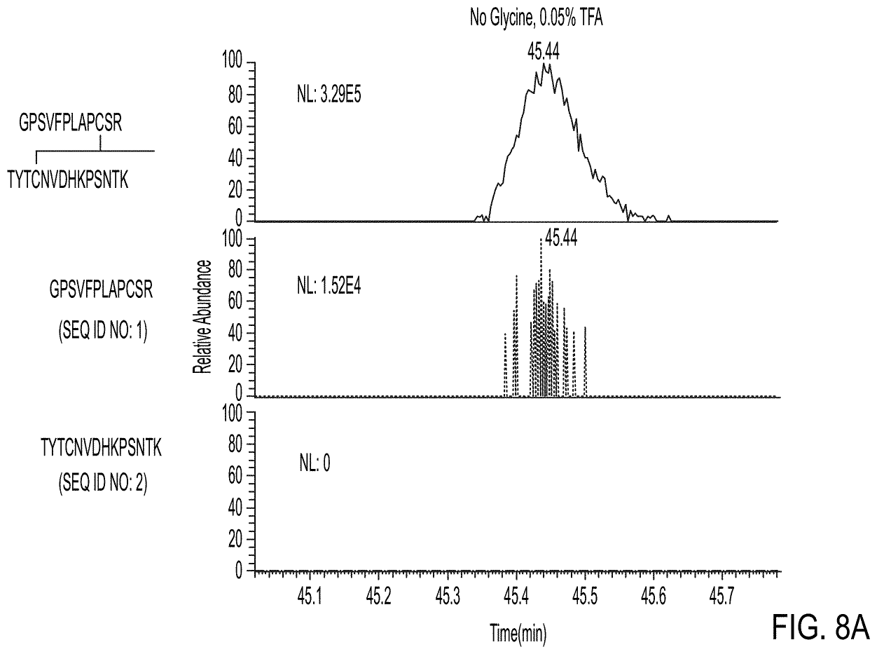

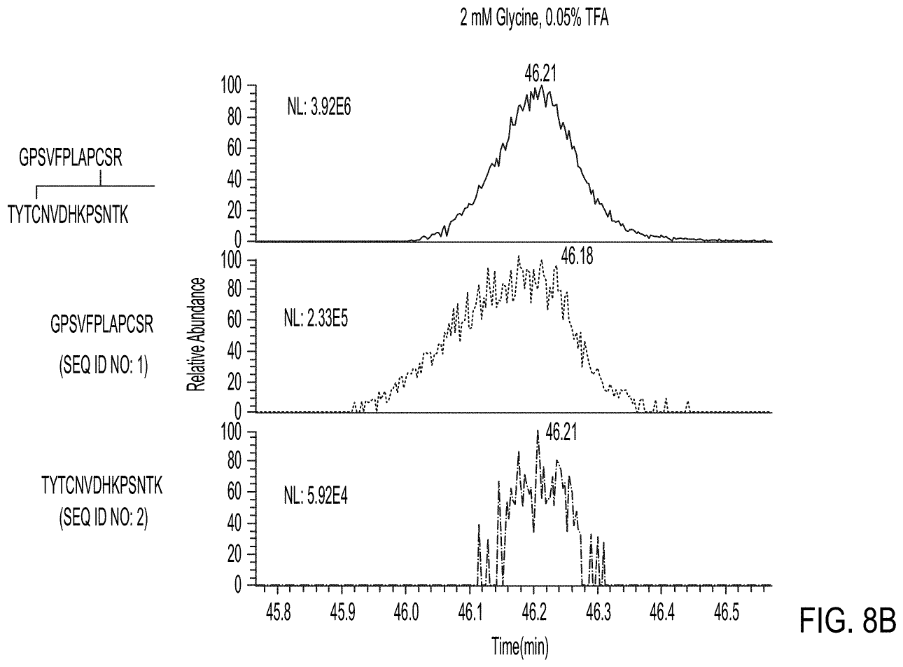

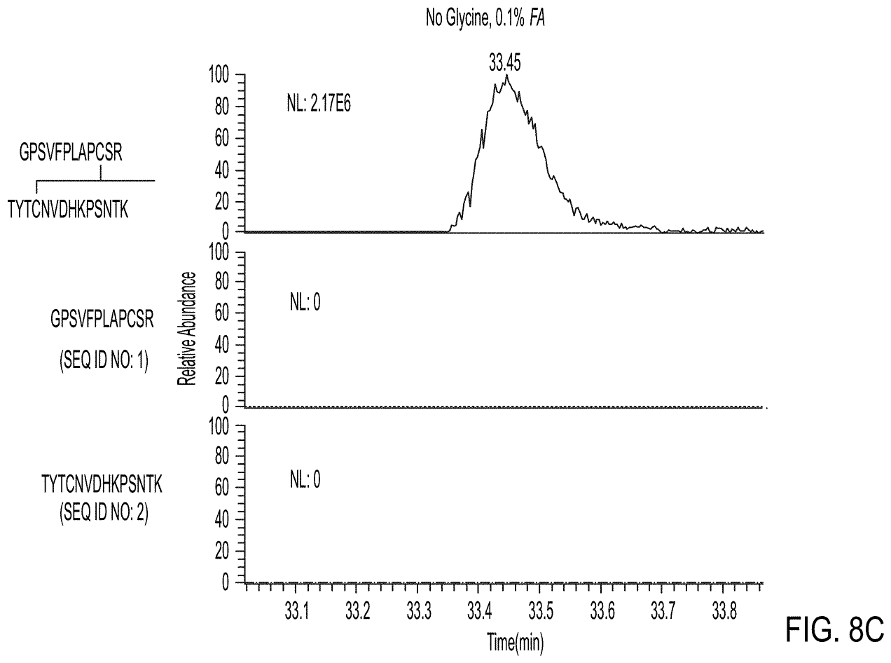

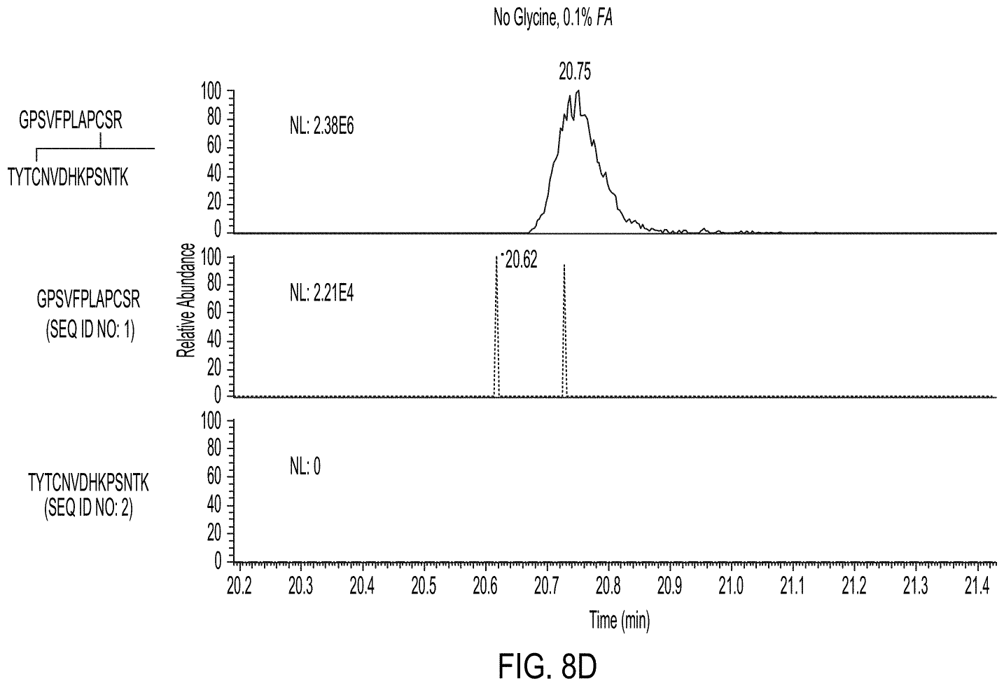

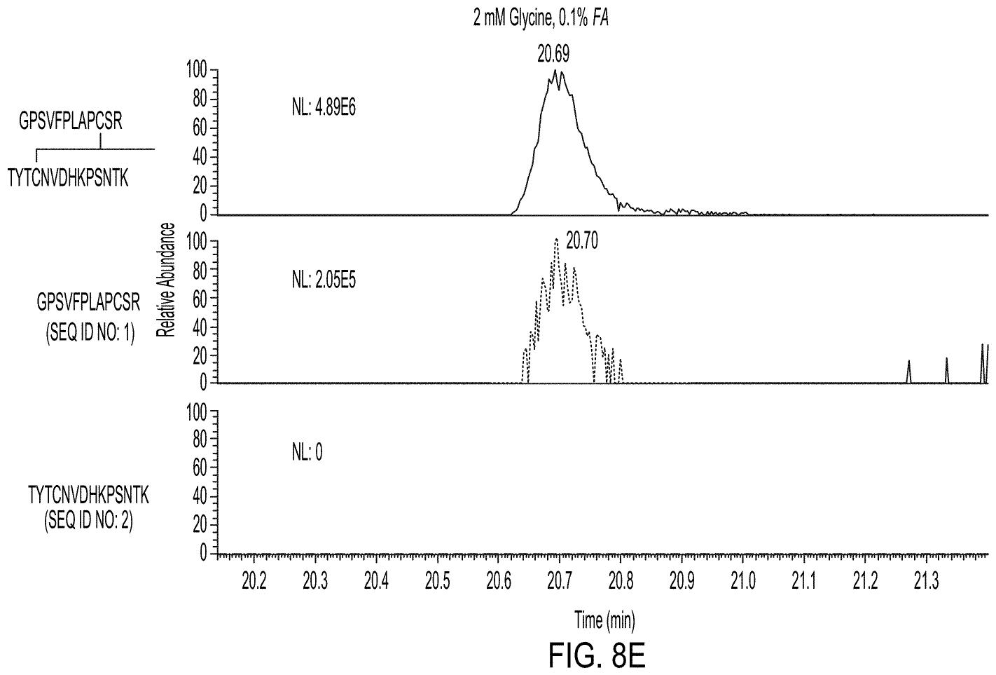

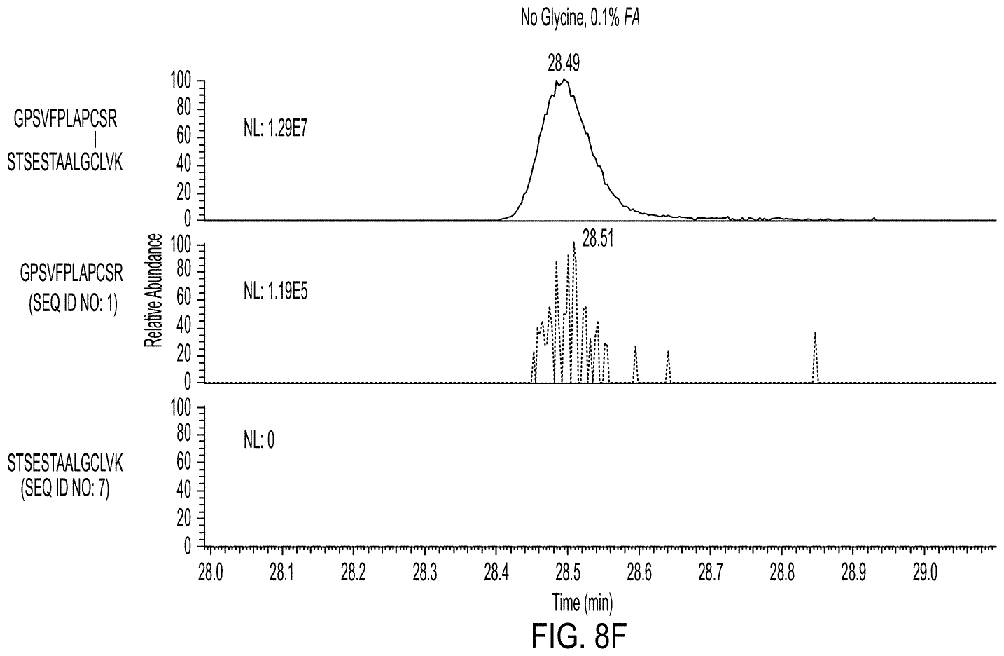

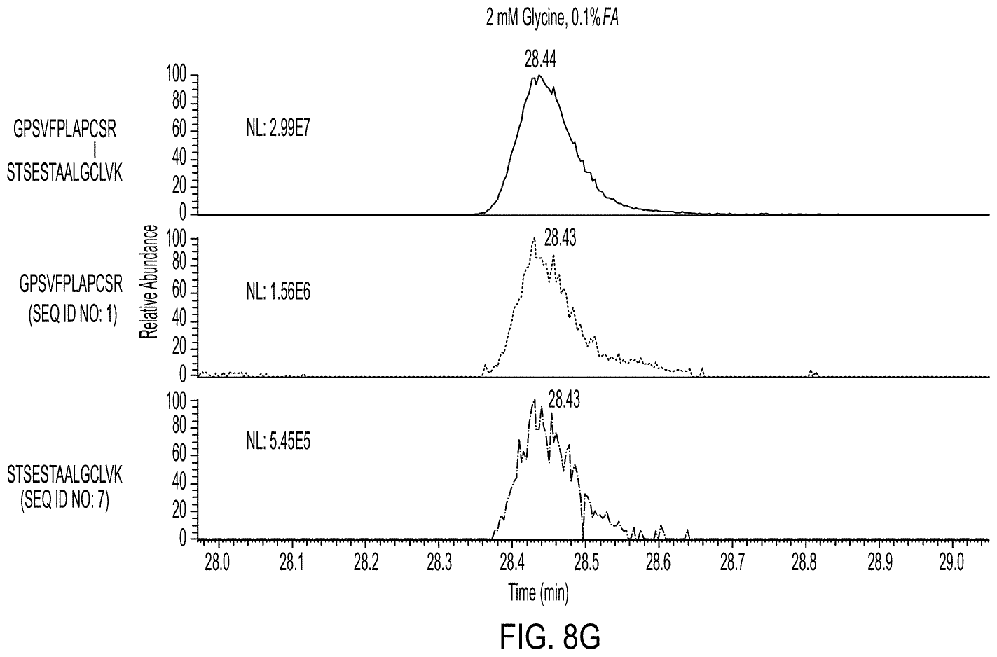

Turning to FIGS. 8A-8B it was observed that glycine improves MS signal of disulfide peptides and corresponding reduced partner peptides by greater than 10× (e.g., 10×-20×) in the presence of 40 μM TCEP and TFA (0.05%). FIGS. 8C-8G depict data illustrating whether FA, with or without glycine, could be used to improve detection of reduced partner peptides. For each of FIGS. 8A-8E, reduced partner peptides correspond to GPSVFPLAPCSR (SEQ ID NO: 1) and TYTCNVDHKPSNTK (SEQ ID NO: 2), which in non-reduced form comprise the disulfide peptide, and for each of FIGS. 8F-8G, reduced partner peptides correspond to GPSVFPLAPCSR (SEQ ID NO: 1) and STSESTAALGCLVK (SEQ ID NO: 7), which in non-reduced form comprise the disulfide. Each of FIGS. 8A-8G show relative abundances of peptide fragments generated from trypsin digestion of mAb2, treated with 40 μM TCEP post-column separation. As illustrated, glycine (e.g., 2 mM) is required in order to detect corresponding reduced partner peptides whether the ion pairing agent is TFA (refer to FIGS. 8A-8B) or FA (refer to FIGS. 8C-8E, and FIGS. 8F-8G).

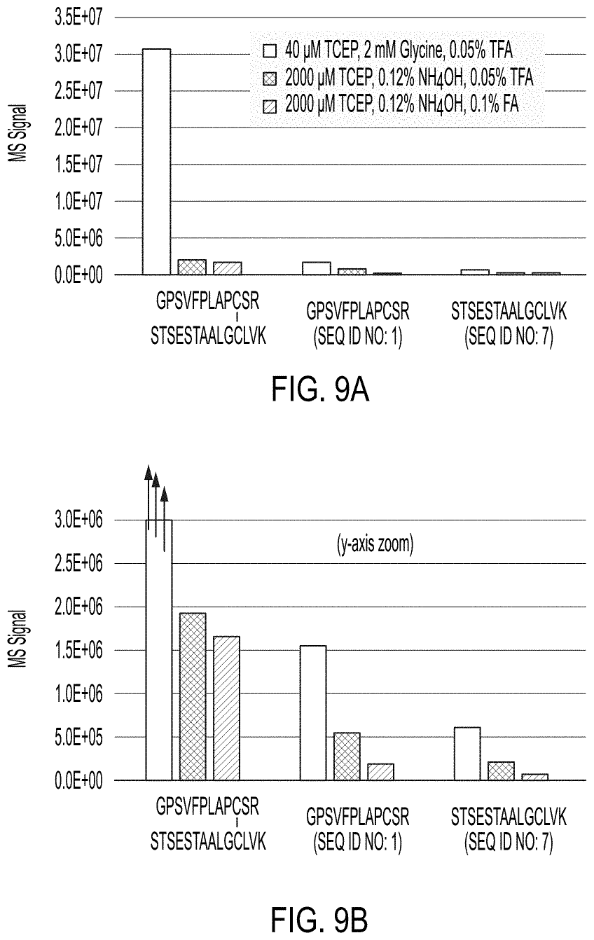

Turning to FIGS. 9A-9B, depicted is MS signal for peptide fragments generated from trypsin digestion of mAb2. The disulfide corresponds to a disulfide of reduced partner peptides GPSVFPLAPCSR (SEQ ID NO: 1) and STSESTAALGCLVK (SEQ ID NO: 7). Same data is depicted at both of FIGS. 9A-9B, with FIG. 9B showing a y-axis zoom of FIG. 9A. The data illustrates that the greatest MS signal for both the disulfide and corresponding reduced partner peptides was observed under conditions where partial reduction was performed with 40 μM TCEP, and where the mobile phase included 2 mM glycine and 0.05% TFA, as compared to samples treated with 2 mM TCEP, 0.12% NH₄OH, and 0.05% TFA, or 2 mM TCEP, 0.12% NH₄OH, and 0.1% FA. The data obtained under conditions where the mobile phase included 2 mM TCEP, 0.12% NH₄OH, and 0.1% FA illustrates that it is possible to detect reduced partner peptides using FA without glycine. However, this appears to require conditions of high concentrations of other components (2 mM TCEP and 0.12% NH₄OH) which, as discussed above, are desirable to avoid in order to obtain improved MS signal. At milder concentrations (e.g., 40 μM TCEP, 0% NH₄OH), detection of reduced peptides requires TFA in the mobile phase.

### Example 2: Optimization of MS/MS Detection of Scrambled Disulfides

The partial reduction strategy discussed above using TCEP (e.g., 20-100 μM) was inferred to result in a typical 1-3% reduction efficiency, this being on top of scrambled disulfides in low abundance to begin with. Accordingly, it was recognized that even with a reasonable MS1 signal, an MS2 scan may be non-existent with Data-Dependent Acquisition (DDA) and/or may be too weak if the MS2 scan occurs at the tail ends of a peak.

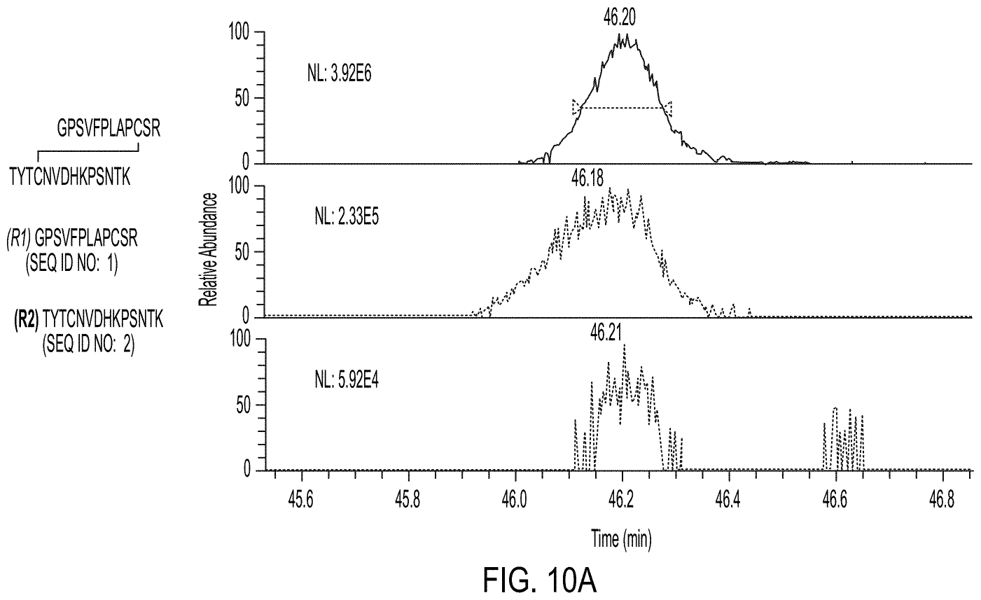

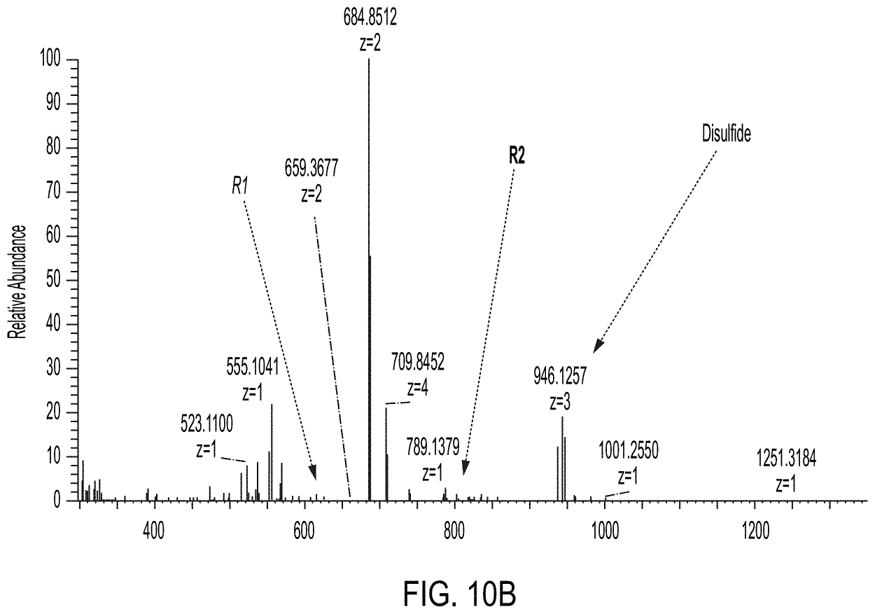

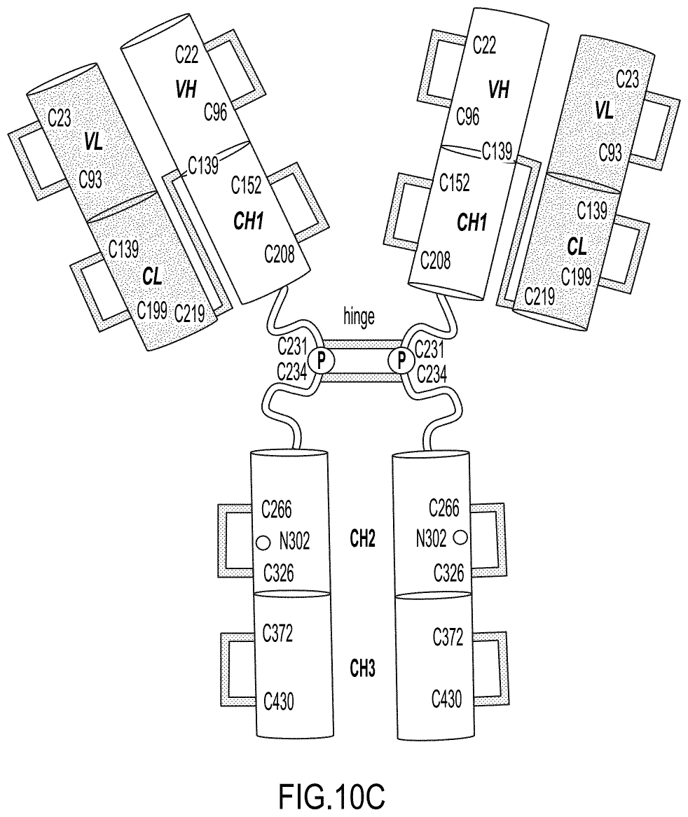

To illustrate these issues, 5 μg of trypsin-digested mAb2 was subjected to tandem mass spectrometry following partial reduction with 40 μM TCEP. The mobile phase included 2 mM glycine and 0.05% TFA. Shown at FIG. 10A are the relative abundances (MS1) of a disulfide peptide and corresponding reduced partner peptides GPSVFPLAPCSR (SEQ ID NO: 1), labeled as R1, and TYTCNVDHKPSNTK (SEQ ID NO: 2), labeled as R2. FIG. 10B depicts relative abundance as a function of m/z for the second stage (MS2) of MS/MS analysis. As shown, even with decent MS1 signal, MS2 signal may be weak or non-existent.

Accordingly, to assure that an MS2 scan exists and is near the apex of each peak, a targeted MS2 approach corresponding to all reduced cysteine-containing peptides was developed. Specifically, turning to FIG. 100, depicted is an exemplary illustration of a mAb, that contains 16 unique cysteine residues as shown, which corresponds to 15 tryptic reduced peptides including the hinge. Targeting all disulfide combinations separately without relying on the above-described partial reduction strategy would yield 120 unique disulfide peptides, 8 of which are native. Hence, by performing partial reduction, it is only necessary to target ˜15 reduced tryptic peptides that contain cysteine. Accordingly, the methodology may comprise building a parallel reaction monitoring (PRM) inclusion list with ˜15 peptides, and scanning across the entire gradient. The number of reduced peptides (e.g., ˜15) is stated as approximate due to omission of the hinge peptide, but also there could potentially be other miscleaved peptides that could be included depending on user preference. Additionally, the MS1 resolution can be lowered for an increased number of MS2 scans.

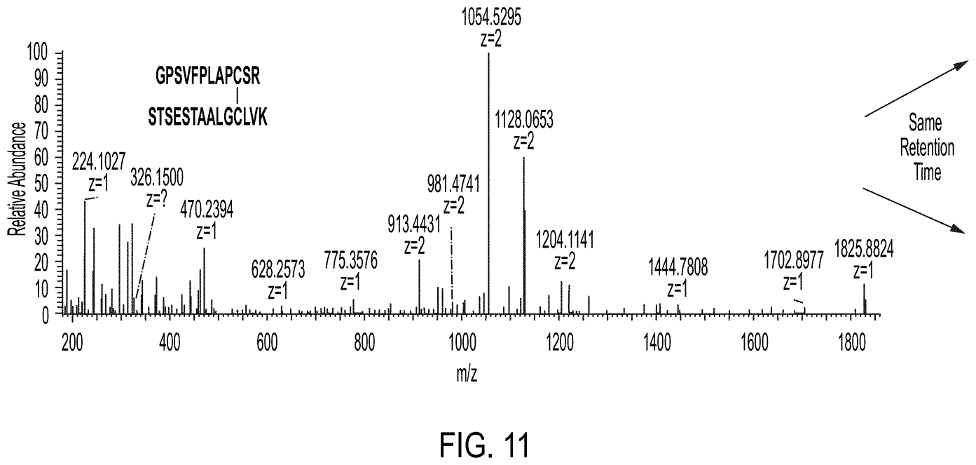

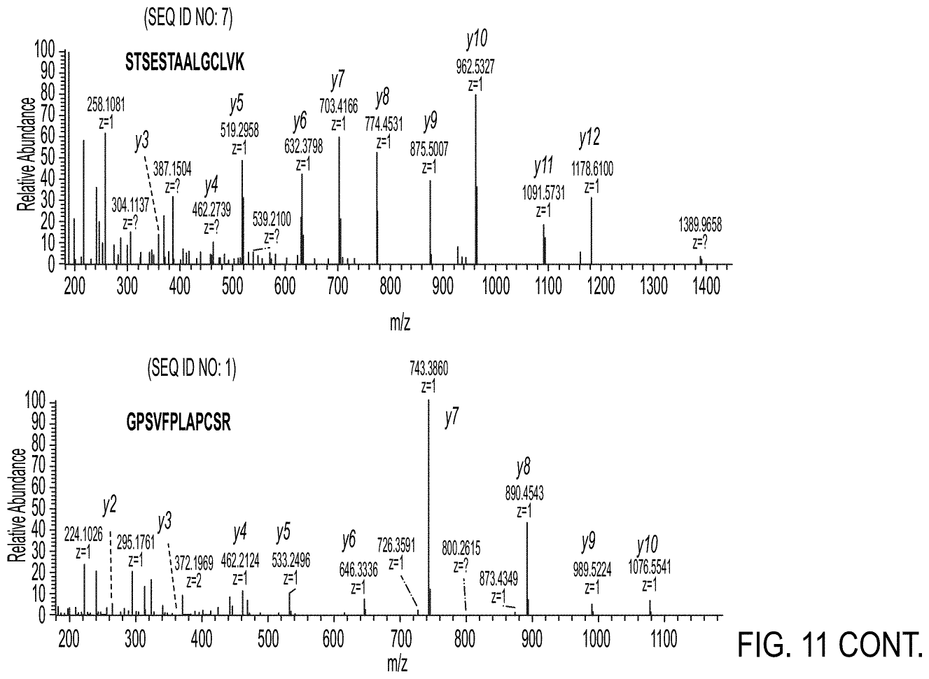

FIG. 11 depicts MS/MS spectra of a disulfide and corresponding reduced partner peptides GPSVFPLAPCSR (SEQ ID NO: 1) and STSESTAALGCLVK (SEQ ID NO: 7). Peptide fragments were generated from trypsin digestion of mAb2. The data depicted at FIG. 11 illustrates that MS/MS spectra of disulfides contain a large degree of complexity as compared to corresponding reduced partner peptides, and that performing a post-column partial reduction with 40 μM TCEP enables simpler characterization of any detectable scrambled disulfides. Said another way, reduced peptides have a much simpler MS/MS spectra than disulfide peptides, due to the MS/MS spectra of disulfides having increased complexity due to possessing exponentially more possible fragmentations as compared to single peptides.

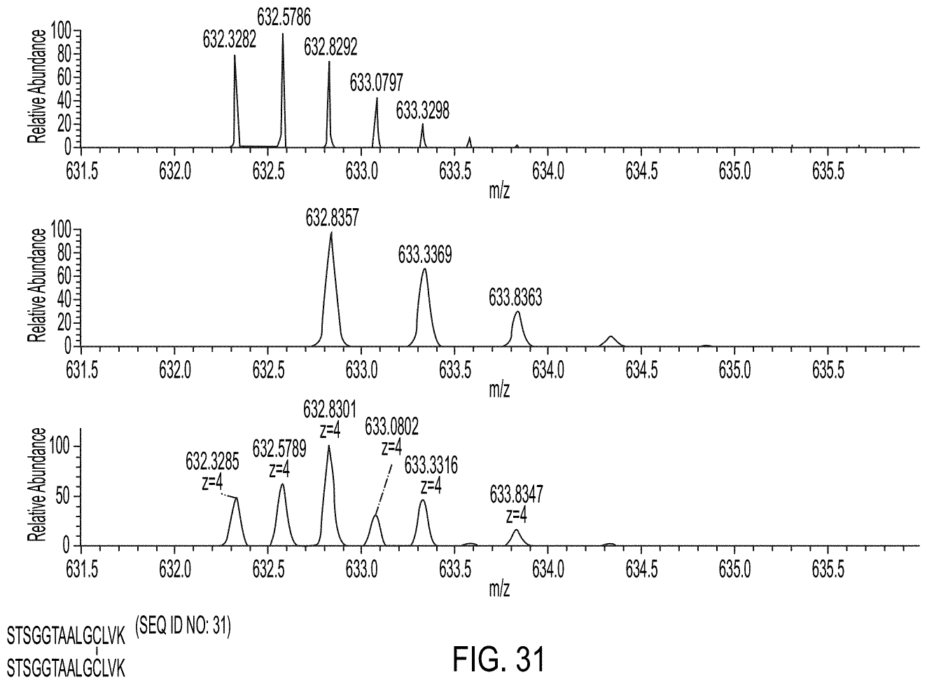

To facilitate MS/MS detection of scrambled disulfides, the following confidence scoring system was developed. The confidence scoring system is comprised of five yes/no queries. The first query judges whether the MS1 mass of a particular disulfide is identified. The second query judges whether the MS1 mass of a first reduced partner peptide is identified. The third query judges whether the MS1 mass of a second reduced partner peptide is identified. It may be understood based on this disclosure that the first reduced partner peptide and the second reduced partner peptide, when linked through cysteine residues, comprise the disulfide mentioned in the first query. The fourth query judges whether a byonic MS2 ID of the first reduced partner peptide has a score greater than a predetermined threshold (e.g., >200). The fifth query judges whether a byonic MS2 ID of the second reduced partner peptide has a score greater than another predetermined threshold (e.g., >200). For each query, a “yes” results in one point, and each “no” results in no, or zero, points. The disulfide ID confidence score is thus as follows: 0=not detected, 1=low confidence, 2=medium confidence, 3=high confidence, and 4 or 5=ultra-high confidence. To improve accuracy in the confidence determination, an option may be to run an additional sample without partial reduction (e.g., absence of TCEP) to determine if reduced peptide peaks disappear at the same retention time. A few issues that can complicate such a confidence determination scheme are that short (e.g., 2-3 residue peptides) may not be detectable, and dimer disulfide peptides have a same m/z as their corresponding reduced partner peptides (although see below with regard to FIG. 31).

### Example 3: Quantification of mAb2 Disulfide Scrambling

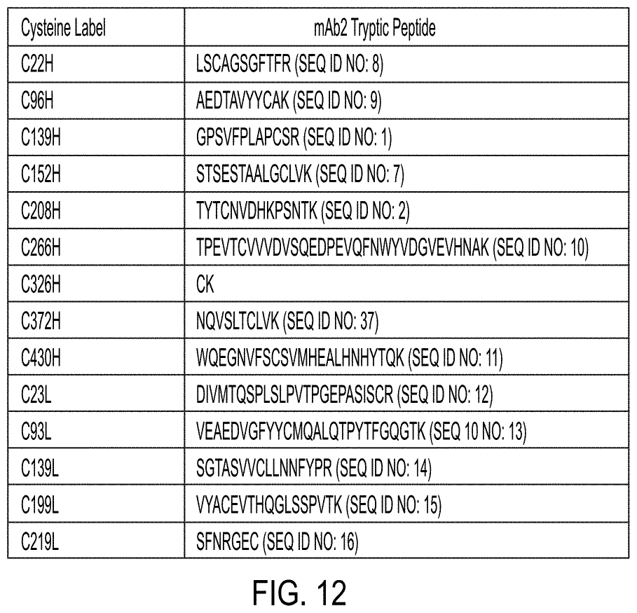

The above-designed targeted MS2 approach to identify and assign confidence values to scrambled disulfides was applied to mAb2. Specifically, mAb2 was subjected to trypsin-digestion under non-reducing conditions, peptide fragments were separated via HPLC, and eluted components were partially reduced via 40 μM TCEP prior to MS/MS analysis (e.g., targeted MS2). 2 mM glycine was used to boost MS signal. FIG. 12 depicts tryptic peptides of mAb2 that contain a cysteine residue, and corresponding cysteine label that includes residue number and designation as to whether the residue is on a light chain (L) or heavy chain (H).

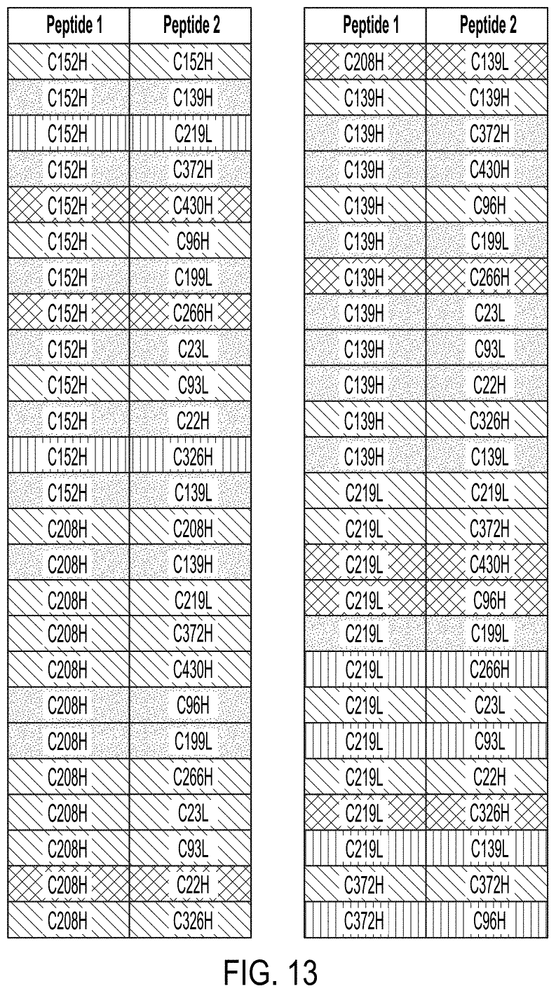

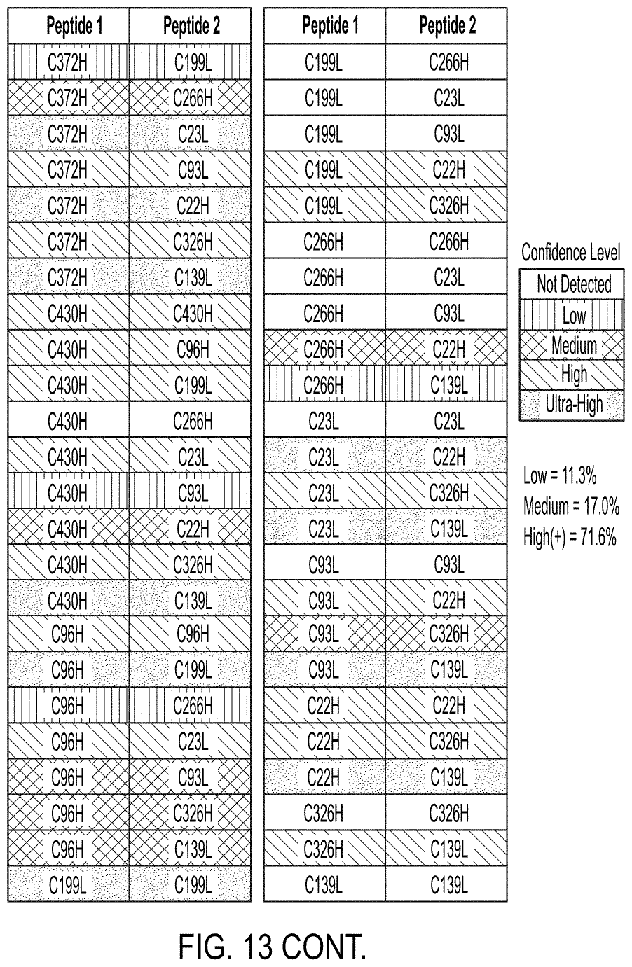

The results are depicted at FIG. 13, which shows all possible scrambled disulfide connections from mAb2, coded by confidence level. 71.6% of scrambled disulfide connections were identified with a high or ultra-high confidence level, 17% were identified with a medium confidence level, and 11.3% were identified with a low confidence level. Scrambled peptides corresponding to the hinge were omitted from the analysis. The data illustrated that the vast majority of all possible scrambled disulfides were identifiable to some extent. The results raised the question of whether the disulfide scrambling could be artificially caused by the protocol used for trypsin digestion. Accordingly, this issue was investigated as discussed below.

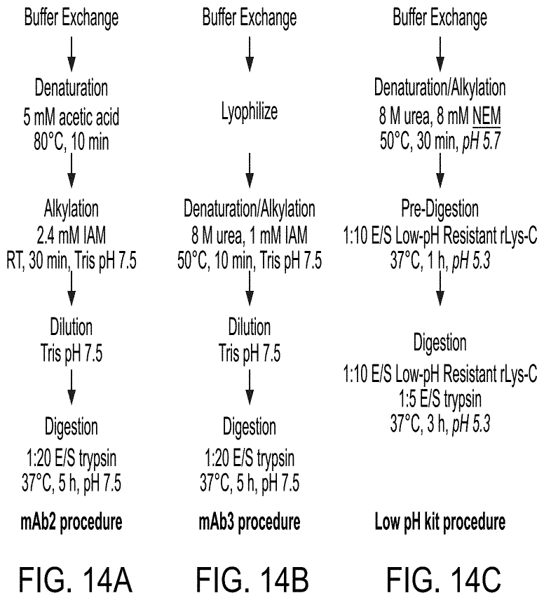

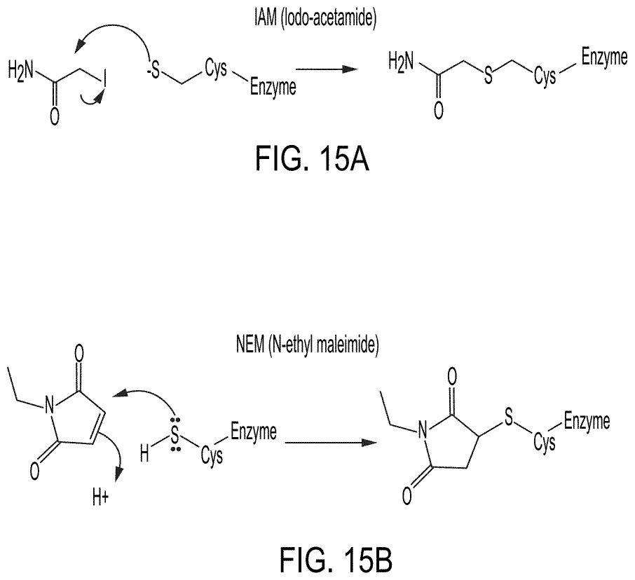

FIGS. 14A-14C depict different digestion protocols corresponding to an mAb2 protocol (FIG. 14A, which was used to generate the data depicted at FIG. 13), an mAb3 protocol (FIG. 14B) and a low pH digestion kit (FIG. 14C) (Promega, Madison, Wis.). As indicated at FIGS. 14A-14C, each protocol includes similar steps (e.g., buffer exchange, denaturation, alkylation, digestion), with some differences as indicated. Each protocol includes an alkylation step, which in the mAb2 and mAb3 protocols comprise alkylation with Iodo-acetamide (IAM), and which in the low-pH digestion kit comprises N-ethyl maleimide (NEM). Structures of IAM and NEM and how they label cysteine residues are shown for reference at FIG. 15A and FIG. 15B, respectively. Other main differences between protocols center on the pH at which denaturation, alkylation and digestion steps are performed. Further, the low-pH digestion kit procedure includes an additional pre-digestion step with recombinant Lys-C protease, prior to the digestion step that additionally includes trypsin. Importantly, the denaturation/alkylation step for the low pH digestion kit procedure is done at a pH of 5.7, the pre-digestion step is done at a pH of 5.3 and the digestion step is done at a pH of 5.3. This is in contrast to similar steps corresponding to the mAb2 and mAb3 protocols, which are carried out at a higher pH (e.g., 7.5).

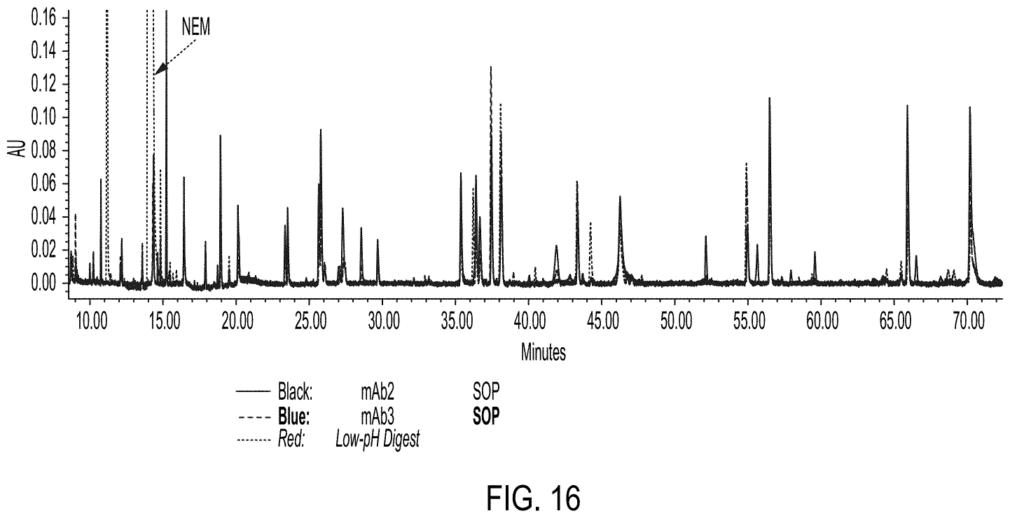

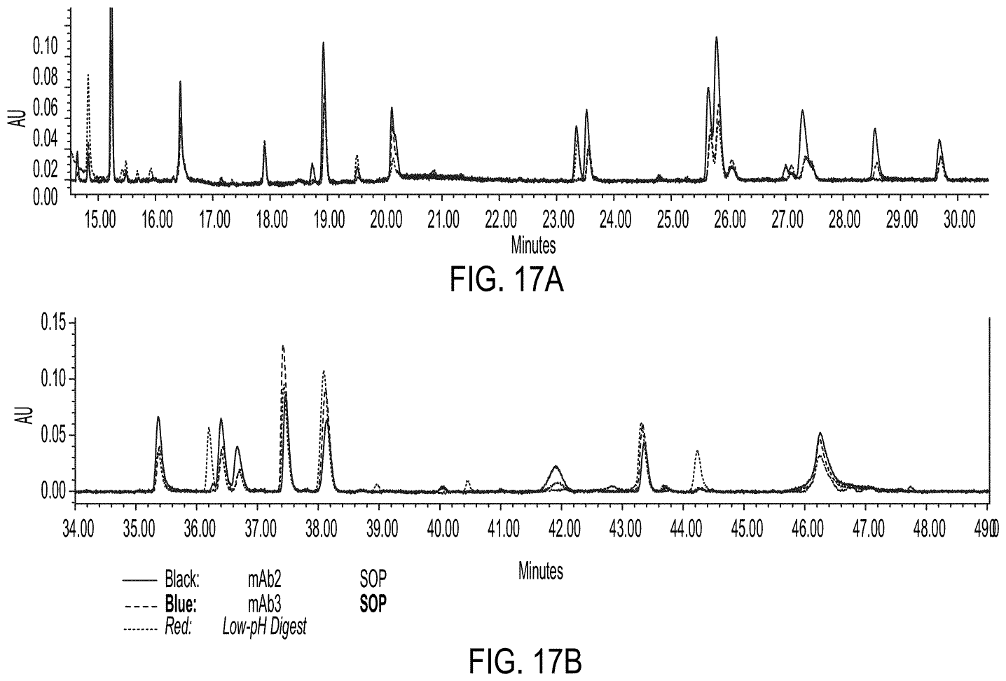

Turning to FIG. 16, a UV chromatograph is shown illustrating that, despite a low pH associated with the low pH kit procedure, the low pH kit procedure yields comparable results to that of the mAb2 and mAb3 procedures. FIG. 16 depicts the UV chromatograph from about 9 minutes to about 72 minutes. FIG. 17A shows a portion of the UV chromatograph to highlight a time window from about 14 minutes to about 30 minutes, and FIG. 17B shows another portion of the UV chromatograph of FIG. 16 to highlight a time window from about 34 minutes to about 49 minutes. Taken together FIGS. 16-17B illustrate that comparable trypsin digestion can be achieved using the lower pH associated with the low pH kit procedure as compared to the higher pH trypsin digestion procedures (e.g., mAb2 and mAb3 procedures). For the UV chromatographs shown at FIGS. 16-17B, individual samples correspond to 5 μg of mAb2 digested via the mAb2 procedure, the mAb3 procedure, and the low pH digestion kit procedure.

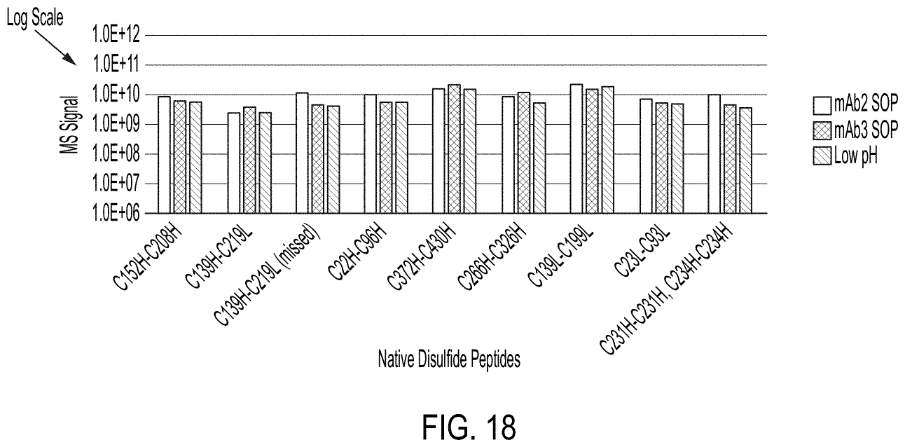

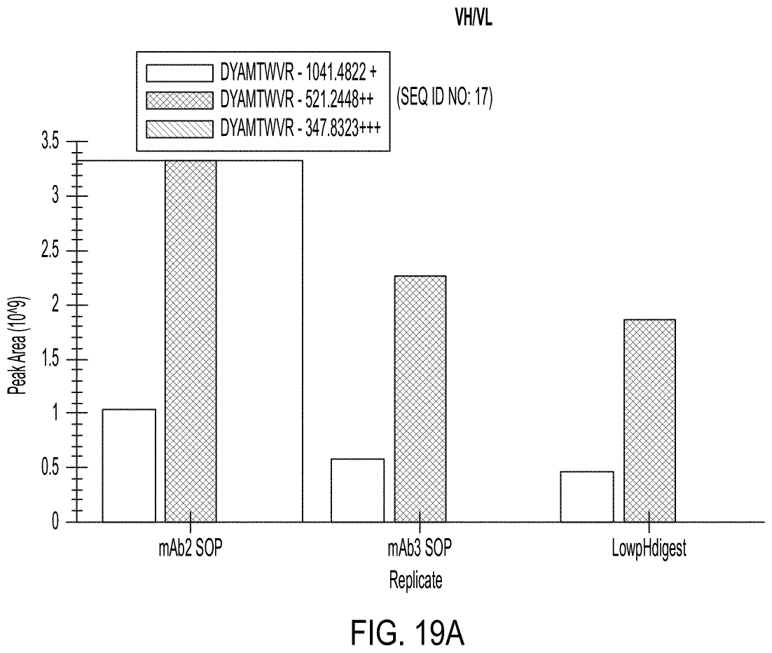

The same samples were used to assess whether there were any discernable differences in MS signal depending on digestion procedure used (e.g., mAb2 procedure, mAb3 procedure, or low pH digestion kit procedure). Turning to FIG. 18, depicted is a graph illustrating MS signal for a number of different native disulfide peptides corresponding to trypsin-digested mAb2 (or trypsin along with low pH resistant recombinant LysC in the case of the low pH digest protocol). As can be seen at FIG. 18, comparable signal intensities were observed for each native disulfide, irrespective of whether the mAb2 procedure, the mAb3 procedure or the low pH digestion kit procedure was used to generate the disulfide peptide fragments.

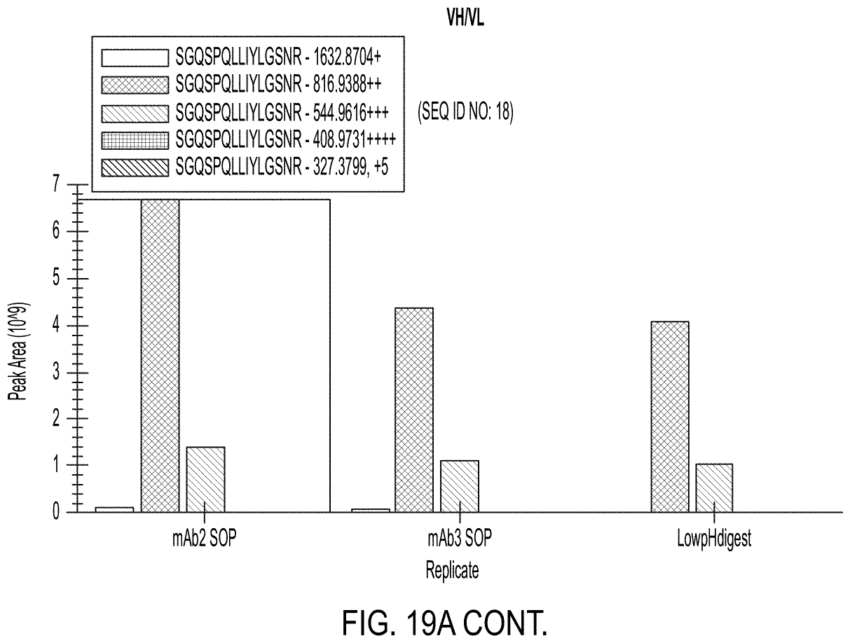

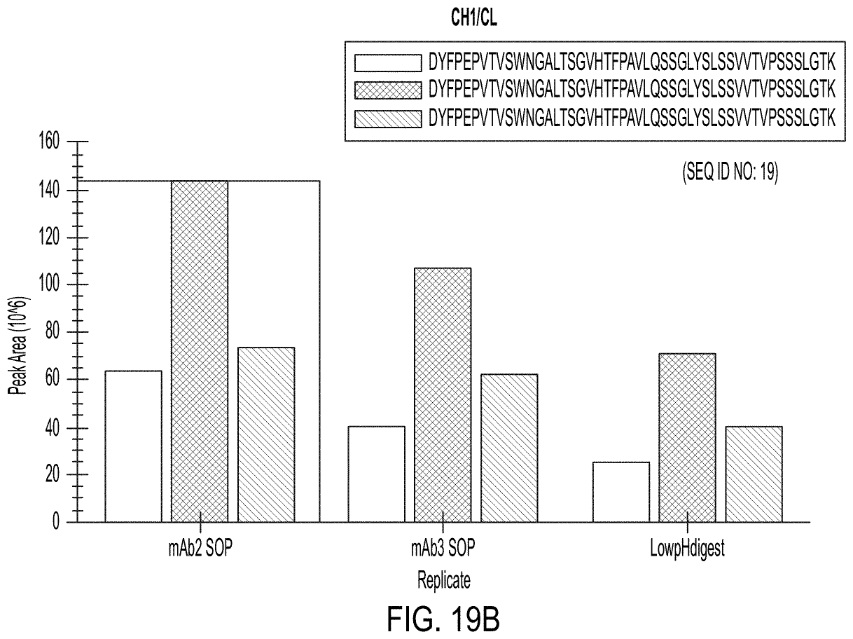

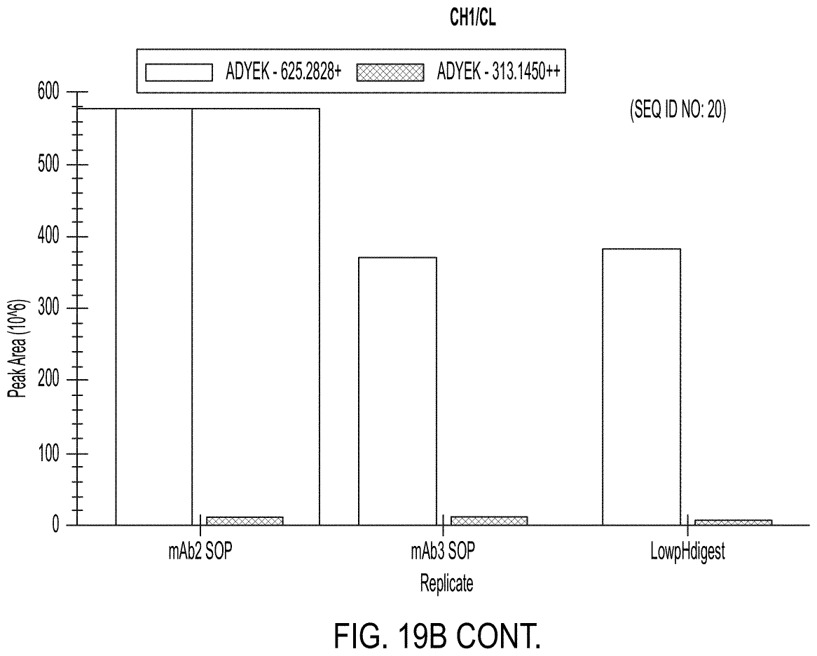

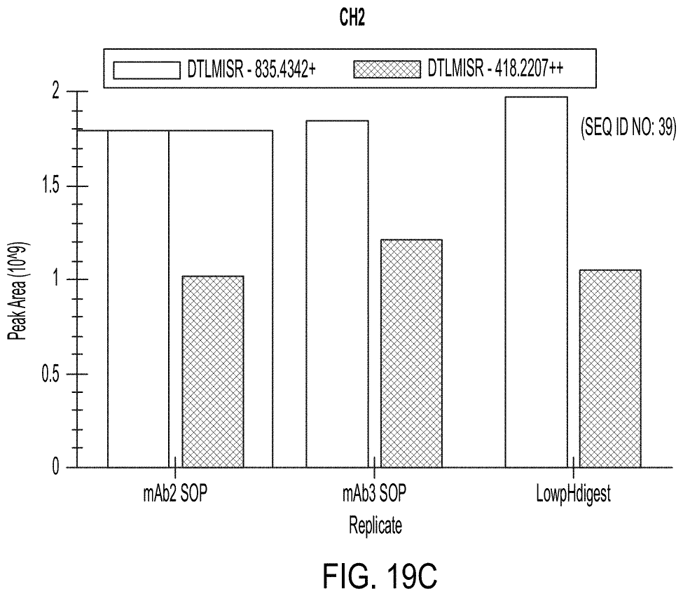

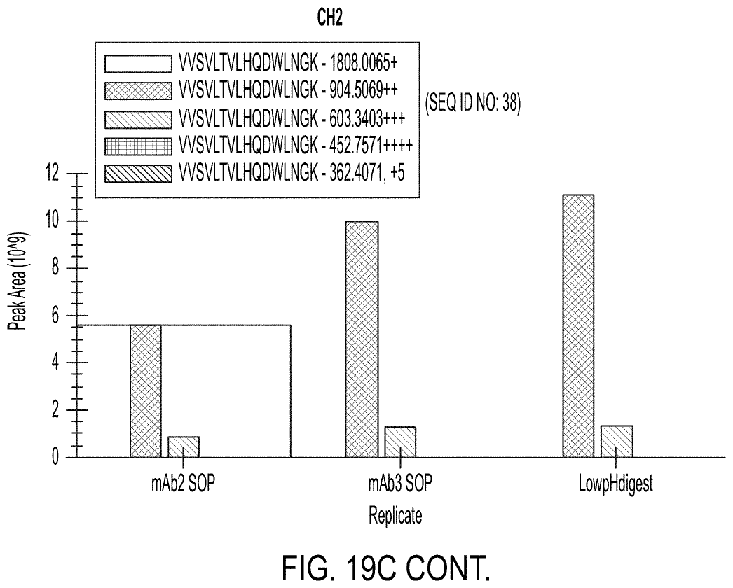

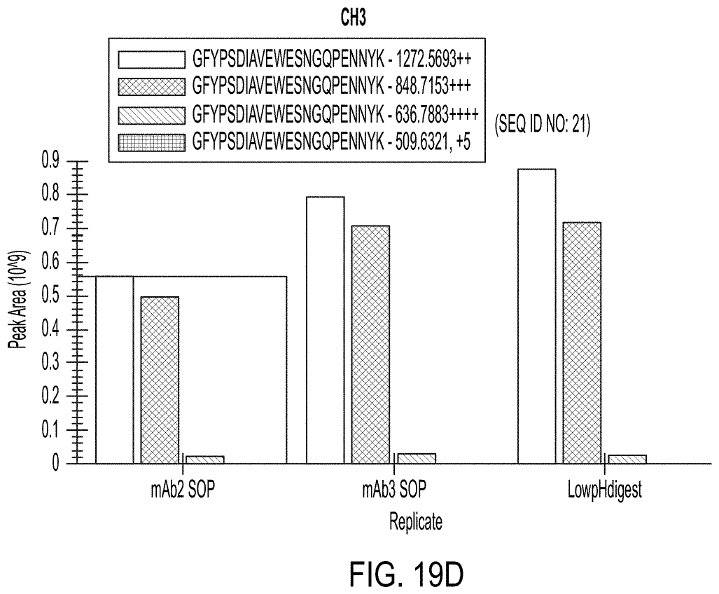

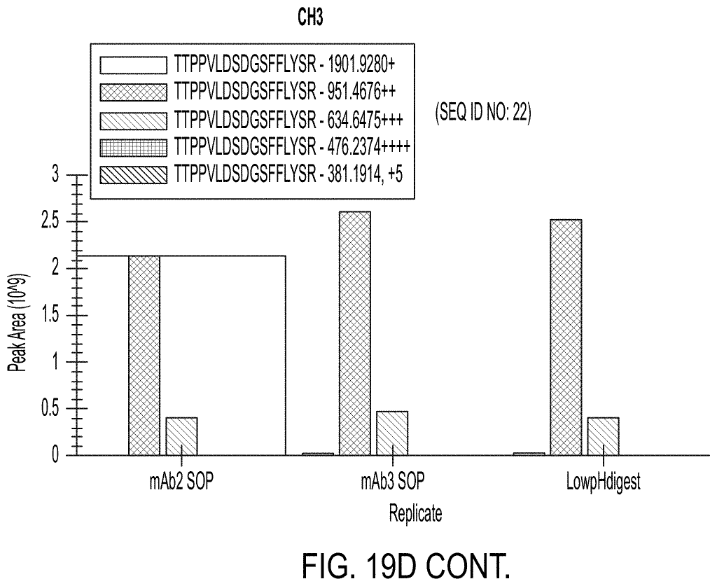

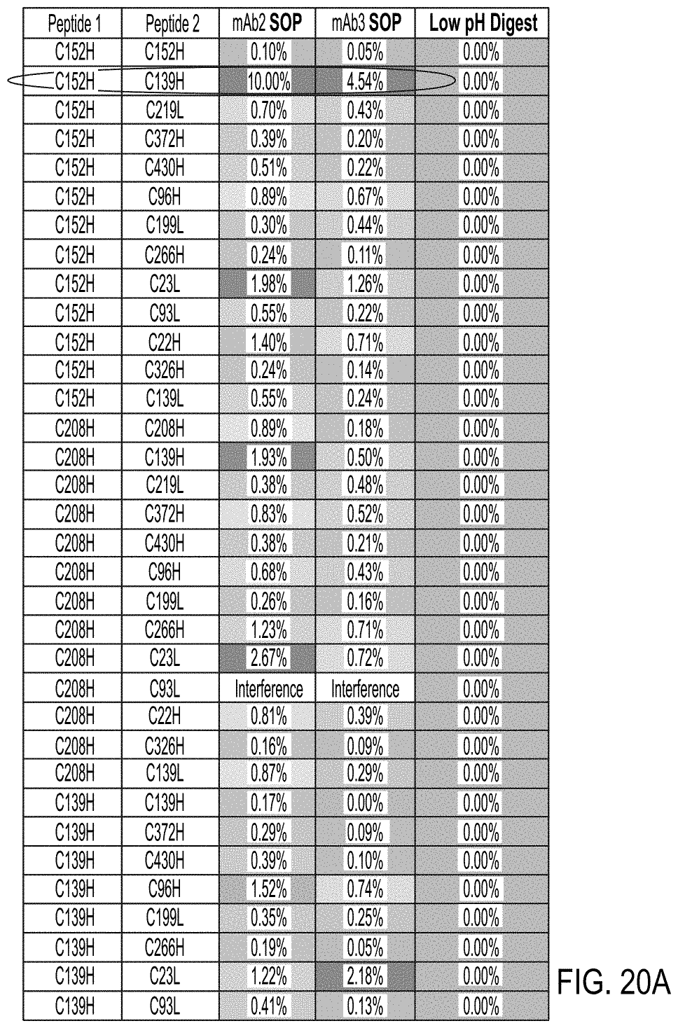

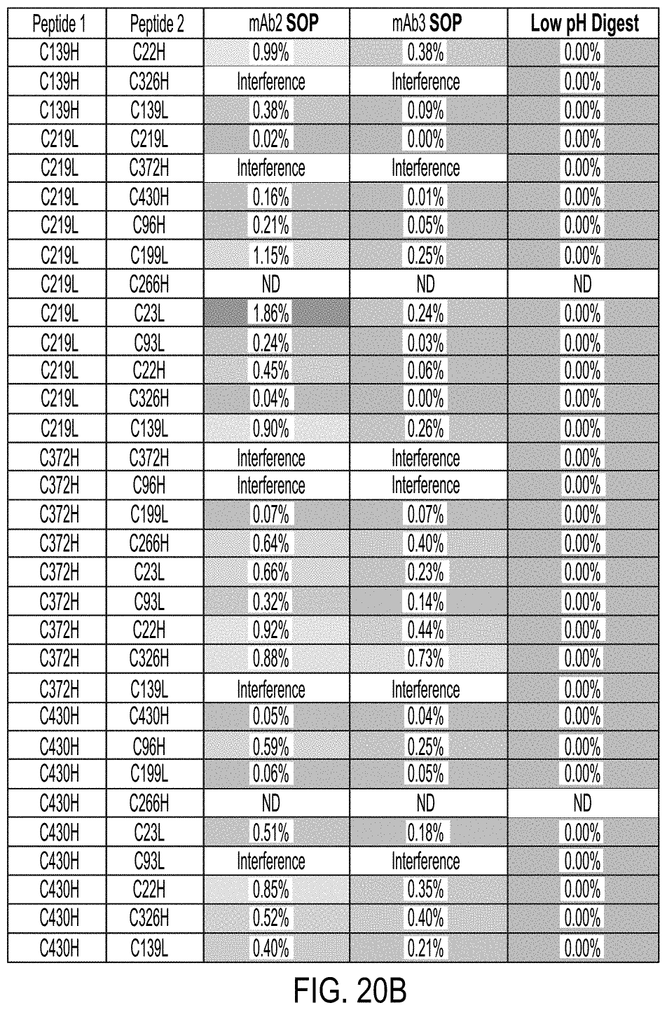

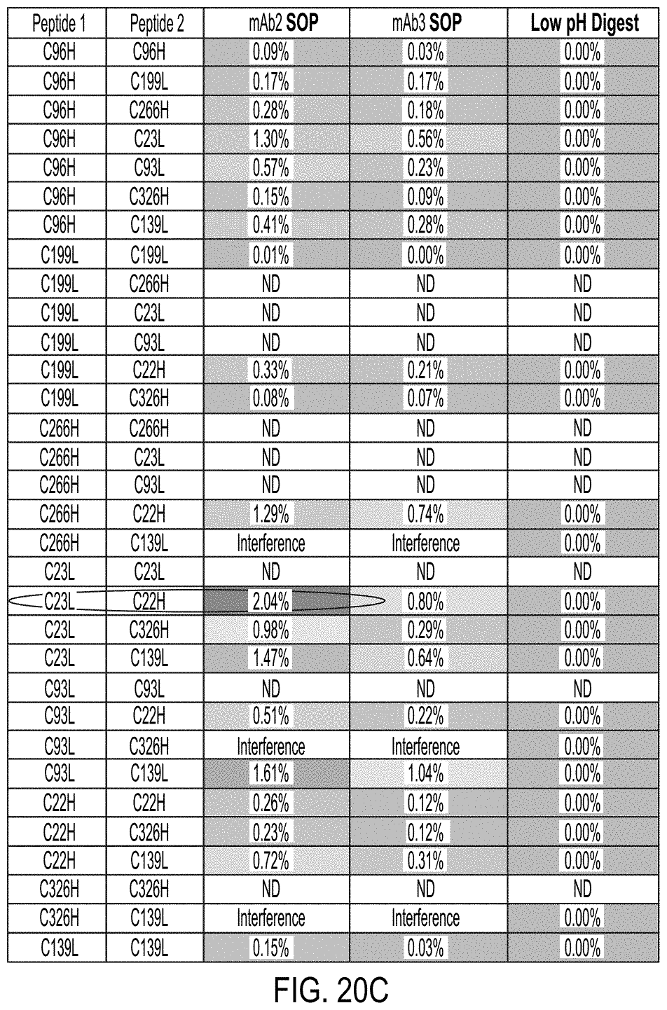

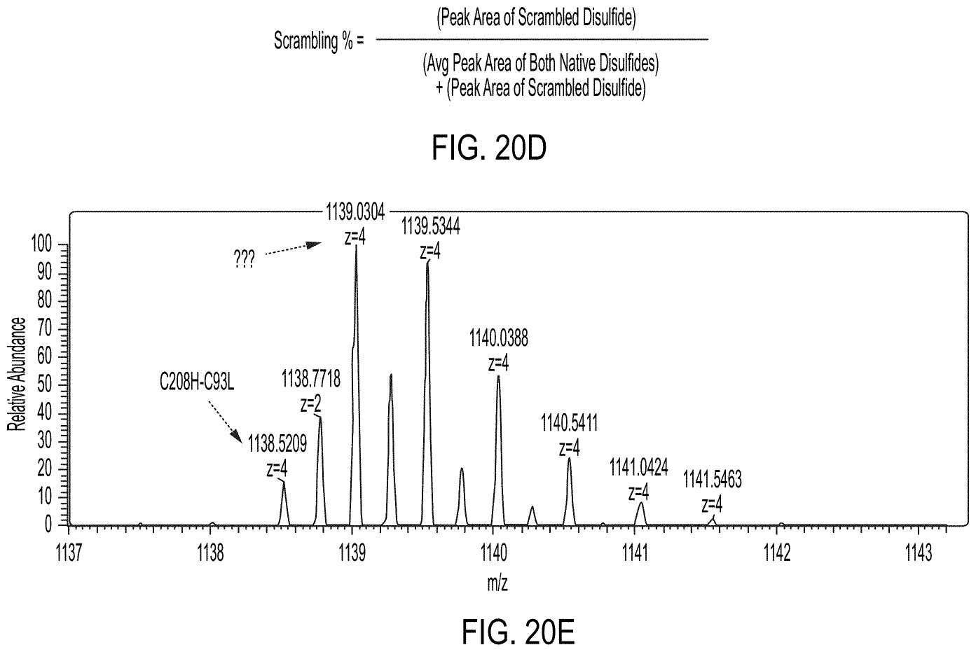

FIGS. 19A-19D illustrate that the low pH digest has a similar digestion efficiency throughout the entire protein compared to the basic digest procedures (e.g., mAb2 and mAb3 procedures). The bars at FIGS. 19A-19D represent MS1 peak integration of non-cysteine-containing peptides within all of the various regions of the mAb (e.g., VH, VL, CH1, CH2, CH3, CL). The data at FIGS. 19A-19D, in combination with the data depicted at FIG. 18, lends support that reported scrambled disulfide levels (refer to FIGS. 20A-20C) are accurate with regard to the low pH digest conditions. Because the data suggested comparable digestion and MS signal level in samples digested under acidic conditions (e.g., low pH digestion kit procedure), it was assessed whether the amount of scrambled disulfides identified initially (see FIG. 13) was artificially caused by the choice of digestion procedure. FIGS. 20A-20C each depict tables illustrating detected disulfide peptides via the procedure discussed above that included the targeted MS2 approach. Shown at FIGS. 20A-20C are all possible scrambled disulfide connections from mAb2, depicted as a heat map corresponding to quantified abundance of scrambled disulfide for each of the three different digestion procedures corresponding to the mAb2 procedure, the mAb3 procedure, and the low pH digestion kit procedure (refer to FIGS. 14A-14C). For each possible scrambled disulfide, a scrambling percentage was calculated as a function of the particular method of digestion (e.g., mAb2 procedure, mAb3 procedure, or low pH kit digestion procedure). The equation for calculating scrambling percentage is shown at FIG. 20D. Briefly, scrambling percentage was determined via dividing a peak area of scrambled disulfide by a sum of the average peak area of both native disulfides and the peak area of the scrambled disulfide. As can be seen at FIGS. 20A-20C, all scrambled disulfides quantified to less than 0.01% when the low pH digestion procedure was used, as compared to higher percentages identified when the higher pH digestion procedures were used (e.g., mAb2 and mAb3 procedures). For some of the disulfides, there was interference, and FIG. 20E shows a representative example of such interference.

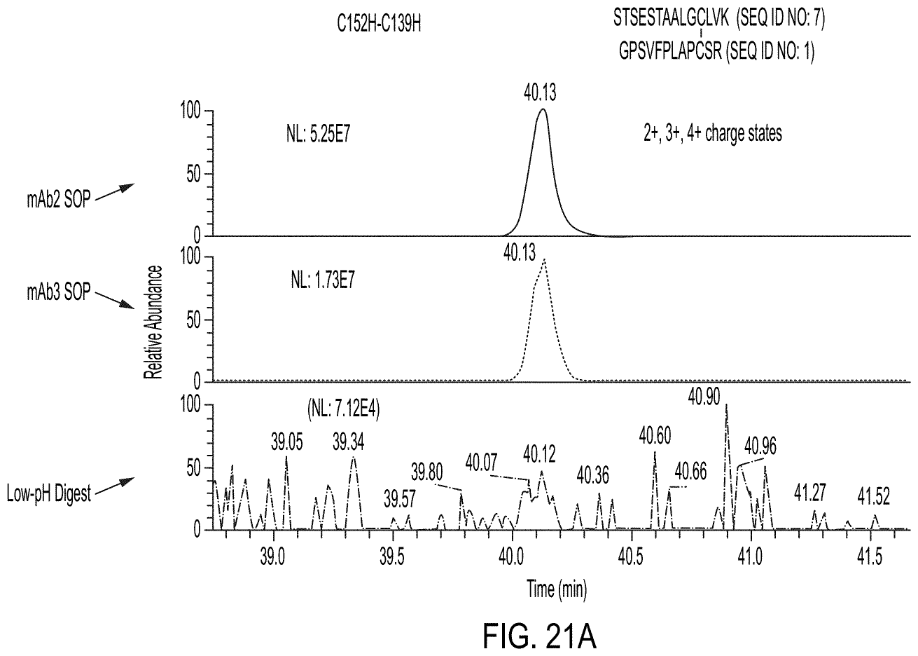

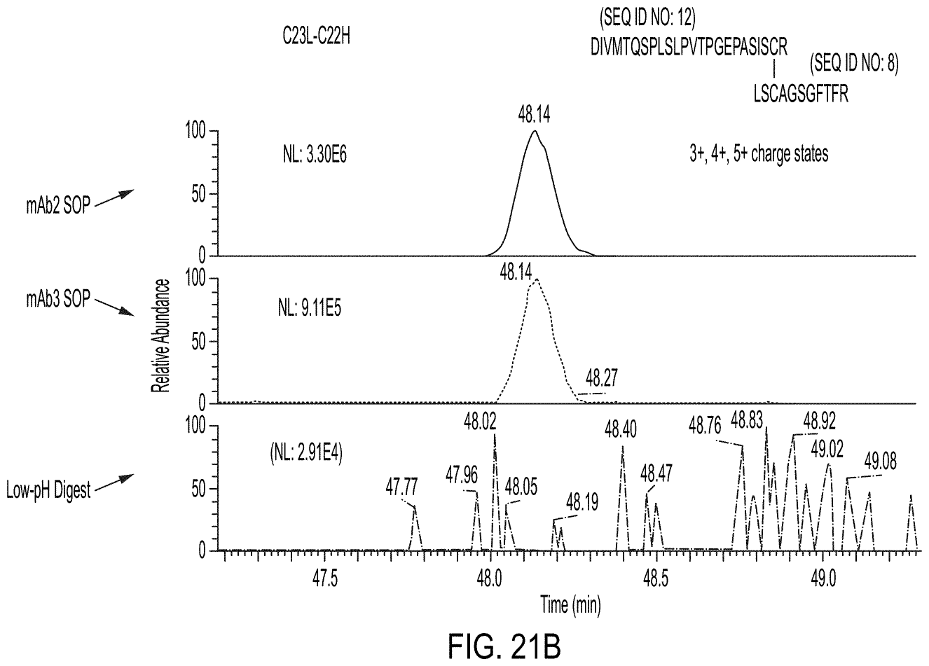

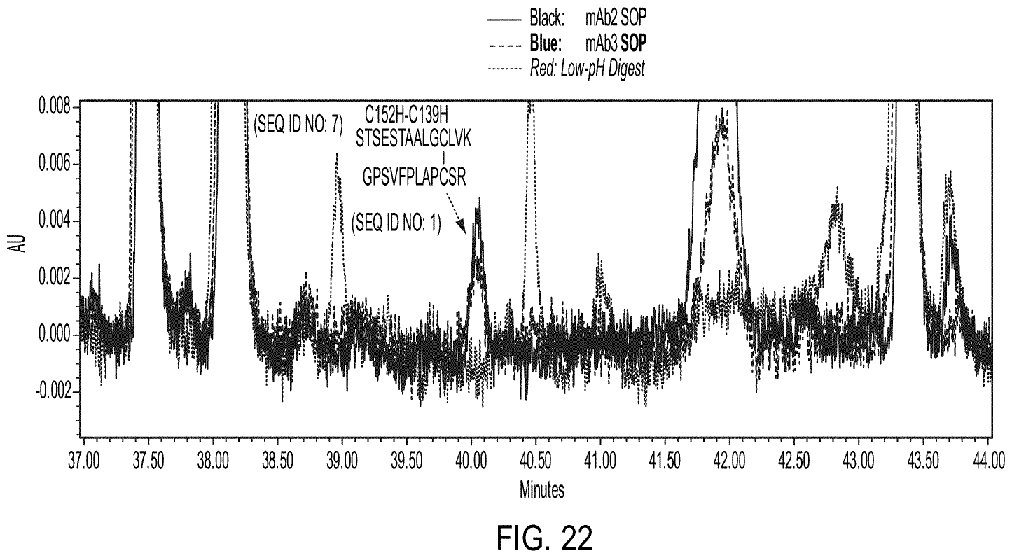

FIG. 20A includes an oval highlighting a particularly high abundance scrambled disulfide identified (C152H-C139H), as does FIG. 20C (C23L-C22H). These were further examined, as discussed with regard to FIGS. 21A-21B. Specifically, FIGS. 21A-21B depict relative abundance of high-abundance scrambled disulfides when the trypsin digestion procedure was done at a higher pH (e.g., mAb2 and mAb3 procedures) or lower pH (low pH digestion kit procedure). FIG. 21A depicts C152H-C139H which corresponds to reduced partner peptides STSESTAALGCLVK (SEQ ID NO: 7) and GPSVFPLAPCSR (SEQ ID NO: 1), and FIG. 21B depicts C23L-C22H corresponding to a disulfide of corresponding reduced partner peptides DIVMTQSPLSLPVTPGEPASISCR (SEQ ID NO: 12) and LSCAGSGFTFR (SEQ ID NO: 8). For each of FIGS. 21A-21B, relative abundances are shown as a function of digestion protocol (mAb2 protocol, mAb3 protocol, or low-pH digestion protocol). As illustrated at both FIGS. 21A-21B, the EICs readily distinguish the high-abundance disulfides from baseline noise when digestion was performed with mAb2 protocol and mAb3 protocol, but the EIC of the scrambled disulfide was not distinguishable from baseline noise for the low-pH digestion condition. For each condition, samples included 5 μg of trypsin-digested mAb2 (or trypsin along with low pH resistant recombinant LysC in the case of the low pH digest protocol) and 2 mM glycine. This observation is consistent with the data shown at FIG. 22, which depicts a UV chromatograph of the C152H-C139H scrambled disulfide (which corresponds to reduced partner peptides STSESTAALGCLVK (SEQ ID NO: 7) and GPSVFPLAPCSR (SEQ ID NO: 1)) which was indicated to comprise a high-abundance scrambled disulfide when either the mAb2 or mAb3 digestion protocols were used to generate the samples. Specifically, FIG. 22 illustrates an absence of a corresponding peak for the sample prepared via the low pH digestion kit procedure, as compared to the observed peaks seen when the samples were prepared via the mAb2 and mAb3 digestion procedures.

### Example 4: Quantification of mAb5 Disulfide Scrambling

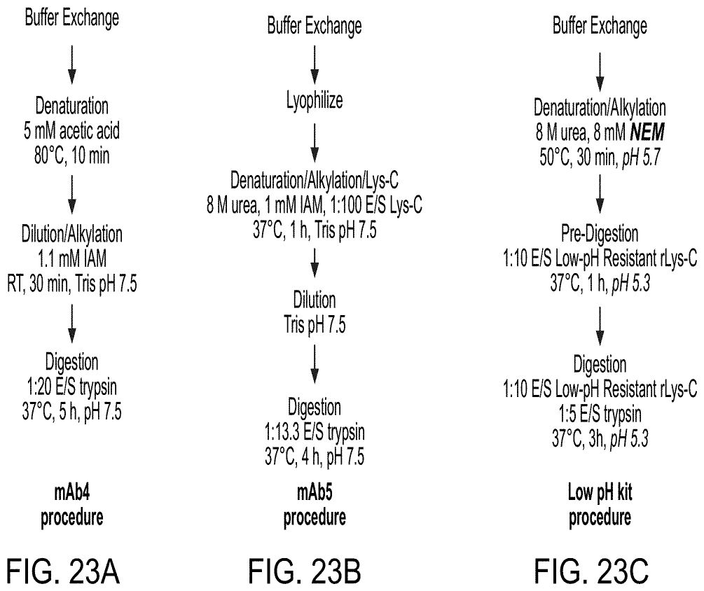

A similar set of experiments as that discussed above with regard to Example 3 was undertaken on another monoclonal antibody, termed herein mAb5. FIGS. 23A-23C depict different digestion protocols examined, specifically, a mAb4 procedure, an mAb5 procedure, and the same low pH digestion kit procedure discussed above and shown at FIG. 14C. The details of each procedure are illustrated at FIGS. 23A-23C, with the main difference being the lower pH of the denaturation/alkylation step (e.g., pH 5.7) and digestion steps (e.g., pH 5.3) associated with the low pH digestion kit procedure as compared to the mAb4 and mAb5 digestion procedures.

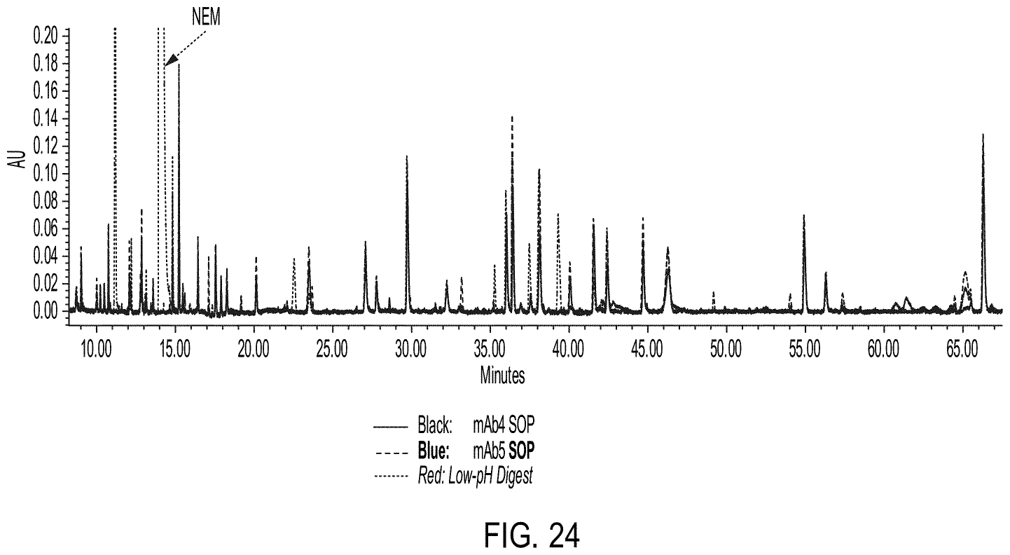

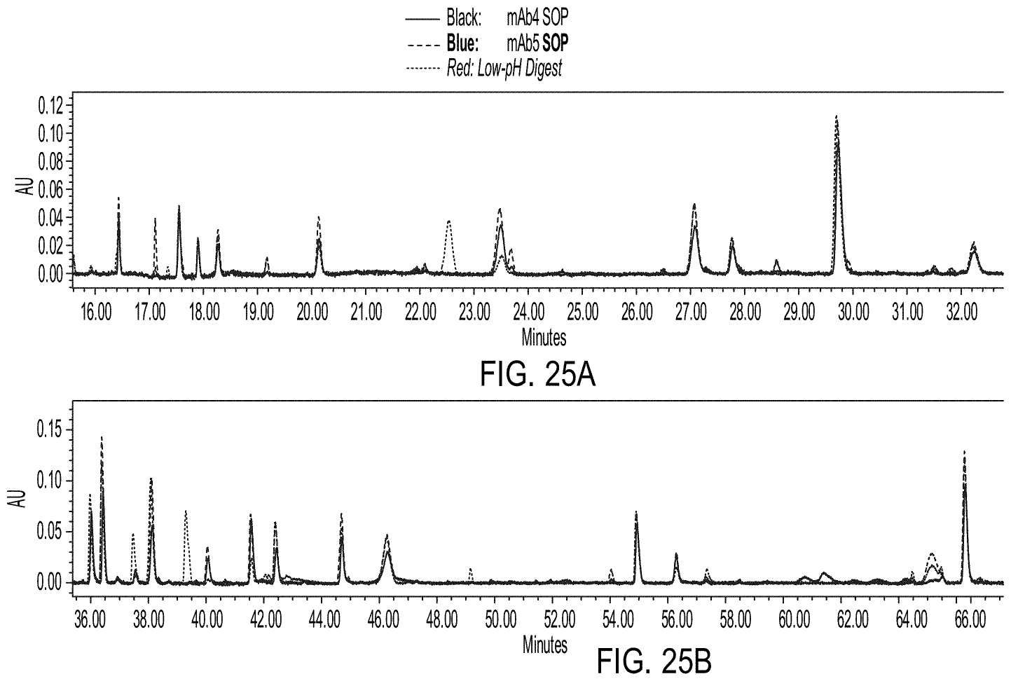

FIG. 24 illustrates an overlay of UV chromatograms corresponding to trypsin-digested mAb5 (or trypsin along with low pH resistant recombinant LysC in the case of the low pH digest protocol) using each of the digestion procedures illustratively depicted at FIGS. 23A-23C (e.g., the mAb4 procedure, the mAb5 procedure and the low pH digestion kit procedure). Similar to that shown above for the mAb2 antibody (see FIGS. 16-17B), FIG. 24 illustrates that despite the low pH associated with the low pH digestion kit procedure, comparable digestion was achieved as compared to digestion using the mAb4 and mAb5 digestion procedures. Samples run to obtain the chromatogram shown at FIG. 24 comprised 5 μg of trypsin digested mAb5 (or trypsin along with low pH resistant recombinant LysC in the case of the low pH digest protocol) in the presence of 2 mM glycine. FIGS. 25A-25B depict portions of the entire chromatograph shown at FIG. 24, for better visual resolution to highlight the fact that digestion of mAb5 was similar irrespective of whether the higher pH digestion conditions (e.g., mAb4 and mAb5 digestion procedures) or the lower pH digestion conditions (e.g., low pH digestion kit procedure) were used.

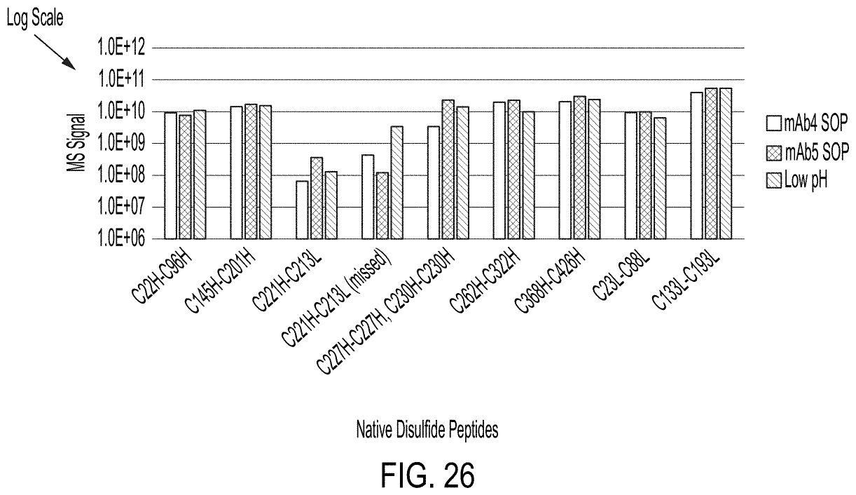

The same samples discussed with regard to FIGS. 24-25B were used to assess whether there were any discernable differences in MS signal depending on digestion procedure used (e.g., mAb4 procedure, mAb5 procedure or the low pH digestion kit procedure). Turning to FIG. 26, depicted is a graph illustrating MS signal (e.g., peak area) for a number of different native disulfide peptides corresponding to trypsin-digested mAb5 (or trypsin along with low pH resistant recombinant LysC in the case of the low pH digest protocol). As can be seen at FIG. 26, comparable signal intensities were observed for each native disulfide, irrespective of whether the mAb4 procedure, the mAb5 procedure or the low pH digestion kit procedure was used to generate the disulfide peptide fragments.

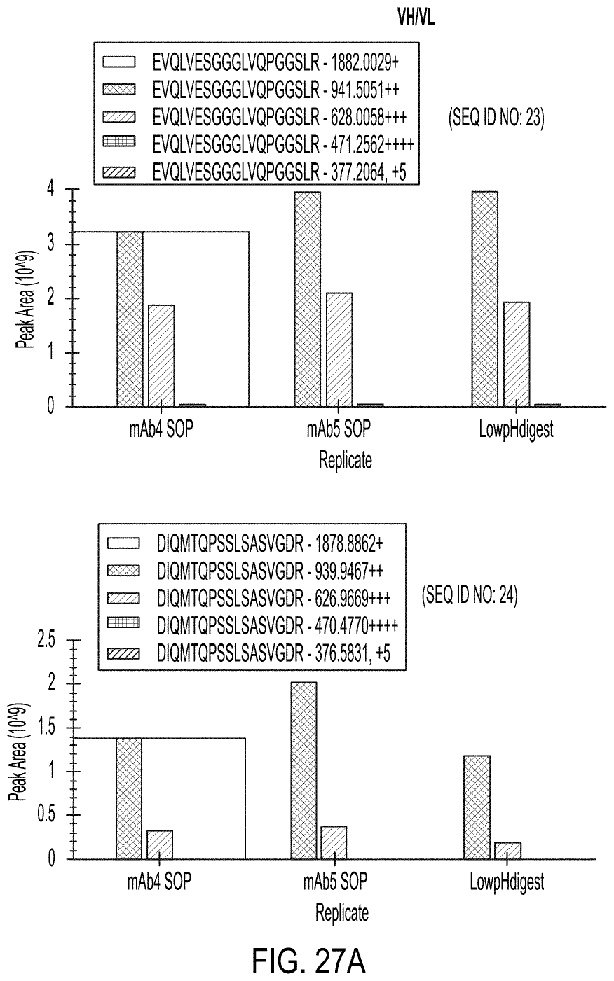

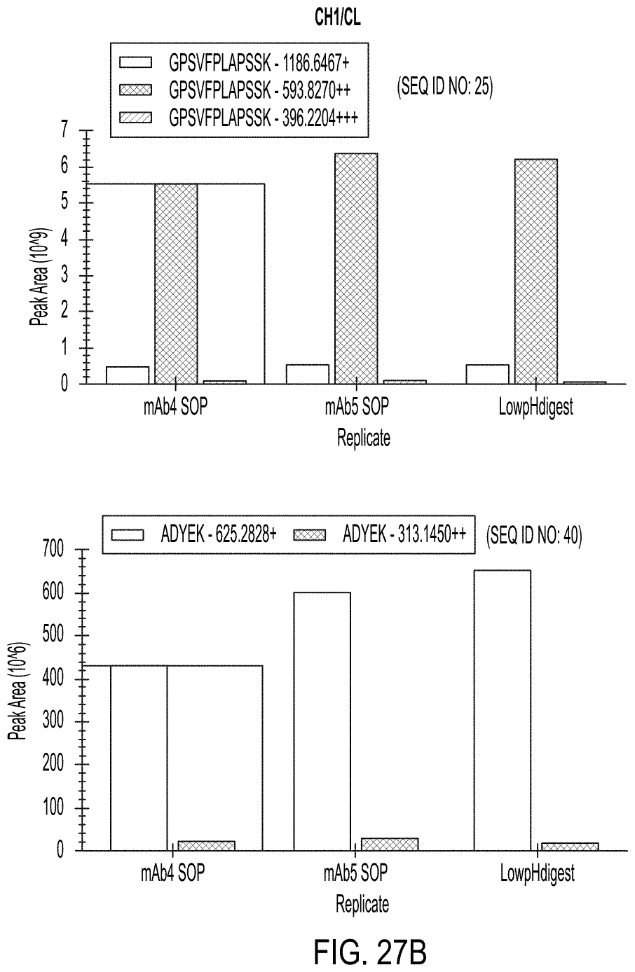

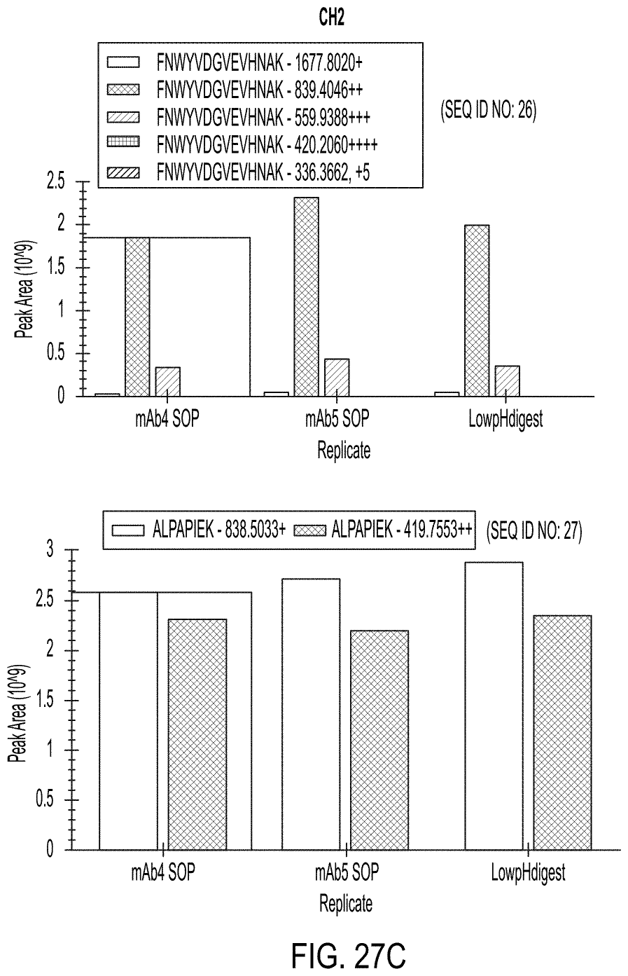

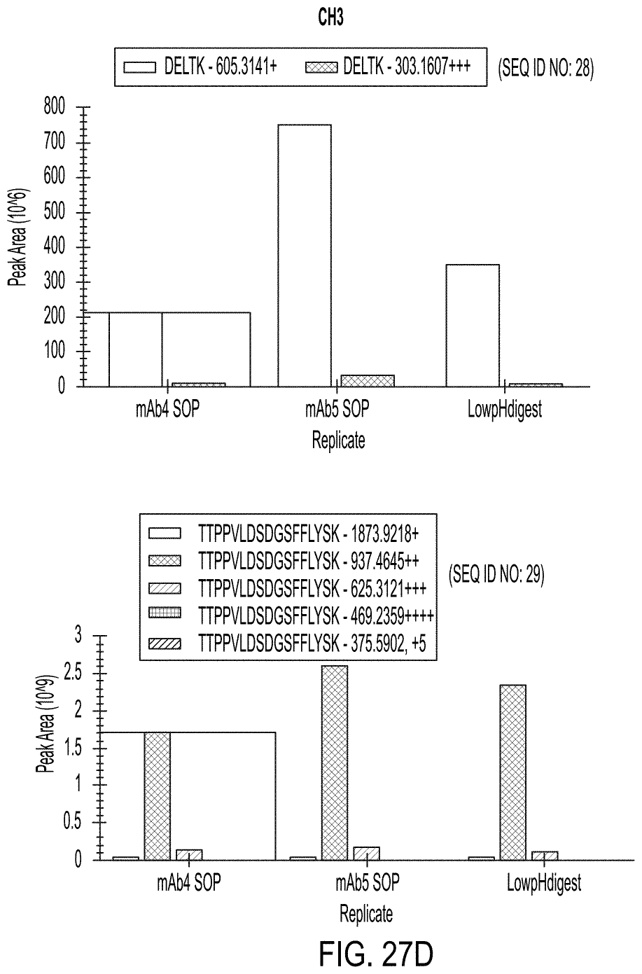

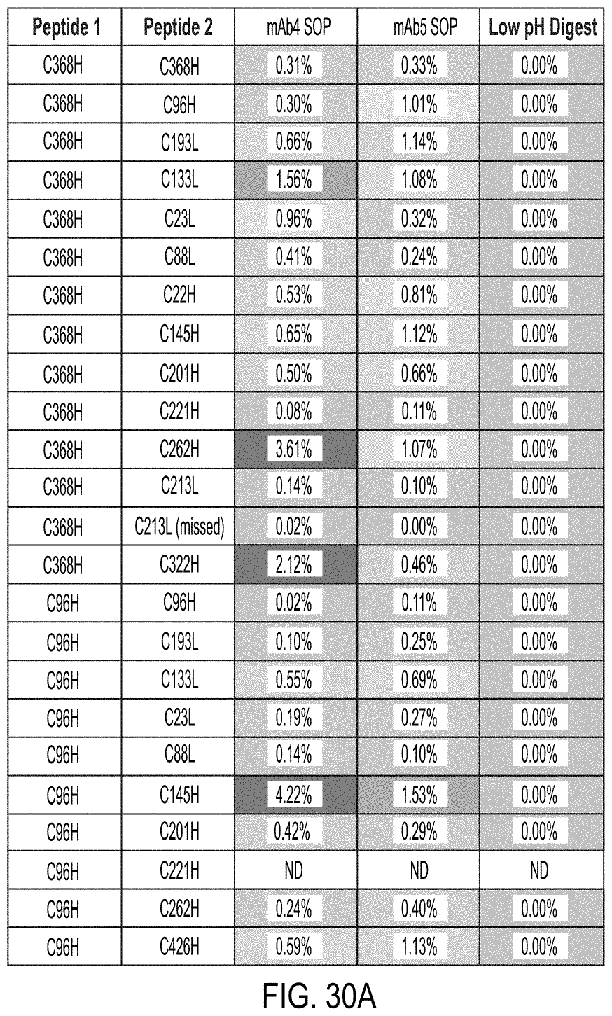

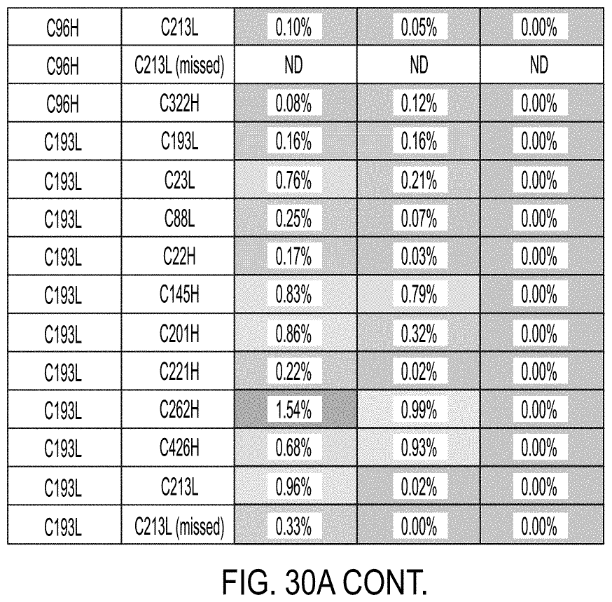

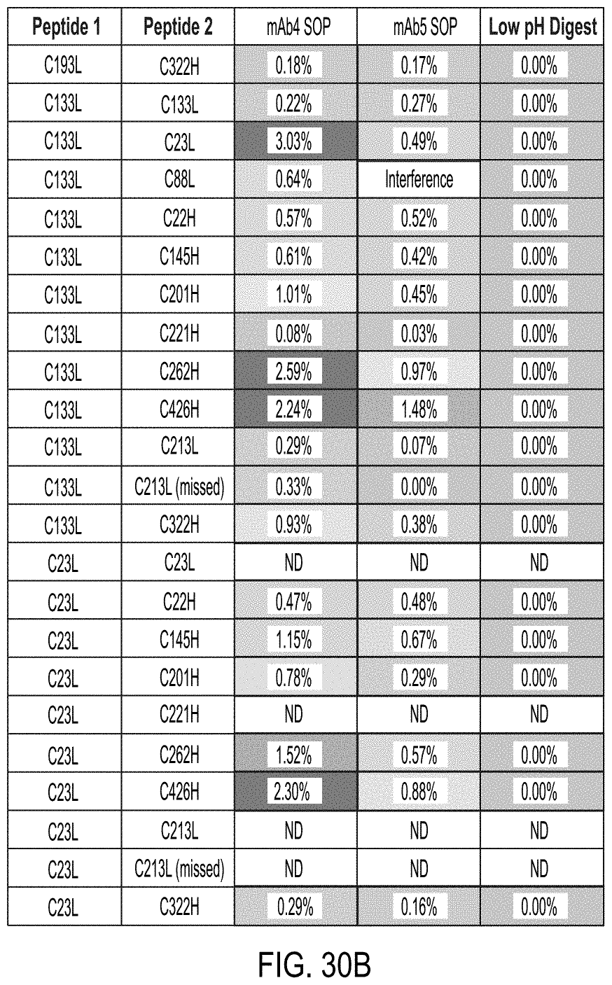

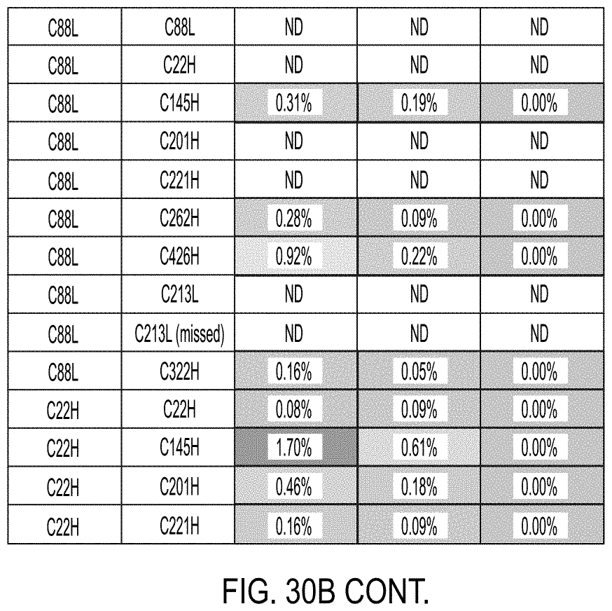

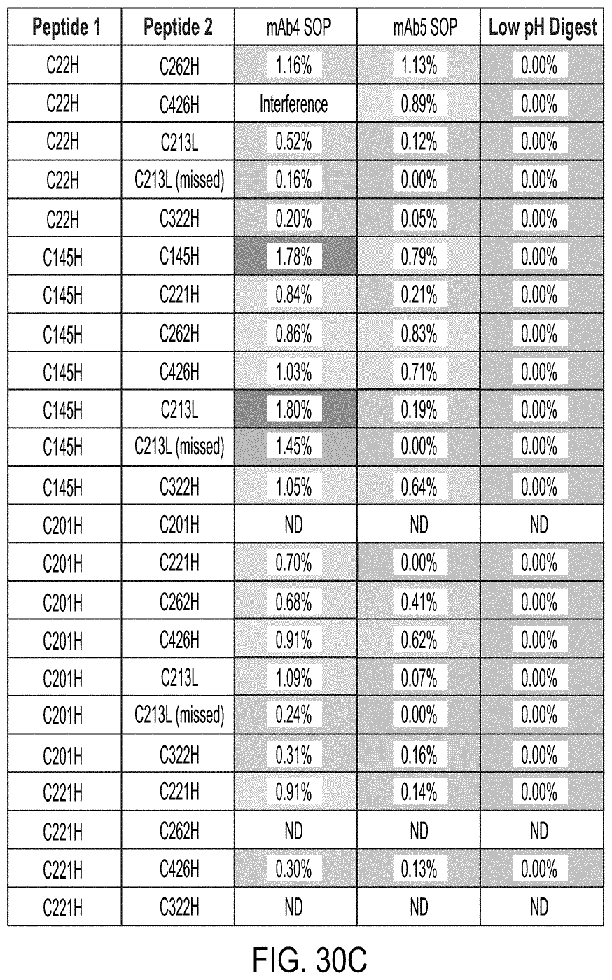

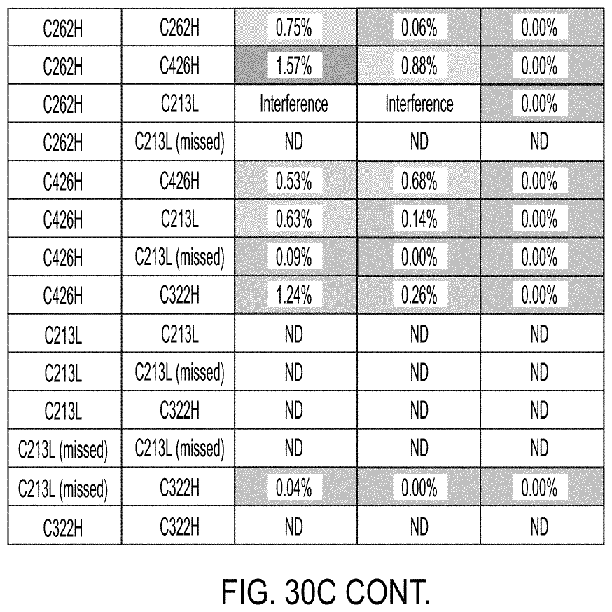

FIGS. 27A-D illustrate that the low pH digest has a similar digestion efficiency throughout the entire protein compared to the basic digest procedures (e.g., mAb4 and mAb5 procedures). The bars at FIGS. 27A-27D represent MS1 peak integration of non-cysteine-containing peptides within all of the various regions of the mAb (e.g., VH, VL, CH1, CH2, CH3, CL). The data at FIGS. 27A-27D, in combination with the data depicted at FIG. 26, lends support that reported scrambled disulfide levels (refer to FIGS. 30A-30C) are accurate with regard to the low pH digest conditions.

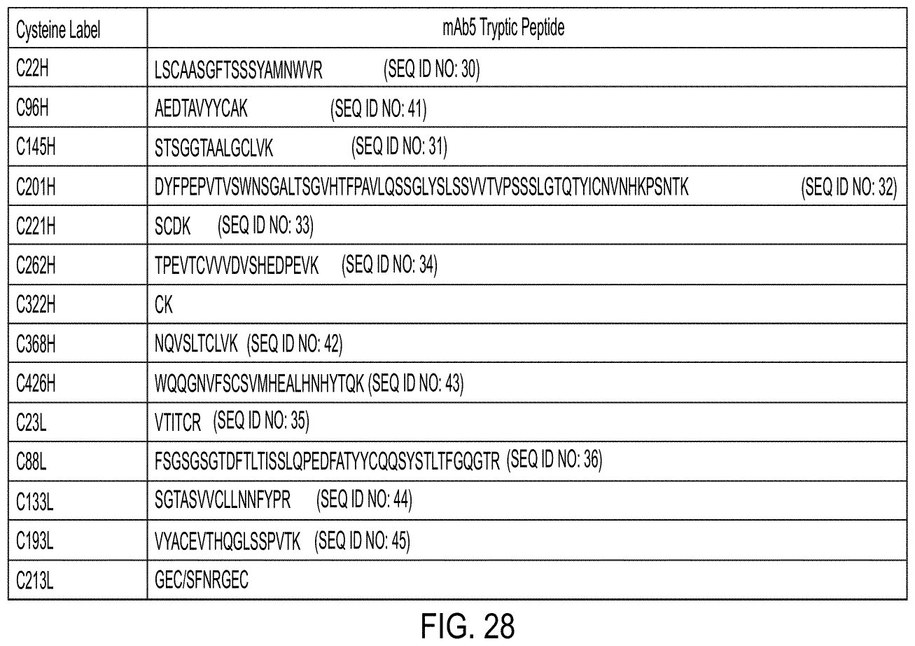

The above-discussed targeted MS2 approach to identify and assign confidence values to scrambled disulfides was applied to mAb5. Specifically, mAb5 was subjected to trypsin digestion under non-reducing conditions using the mAb4 digestion procedure, peptide fragments were separated via HPLC, and eluted components were partially reduced via 40 μM TCEP prior to MS/MS analysis (e.g., targeted MS2). 2 mM glycine was used to boost MS signal. FIG. 28 depicts tryptic peptides of mAb5 that contain a cysteine residue, and corresponding cysteine label that includes residue number and designation as to whether the residue is on a light chain (L) or heavy chain (H).

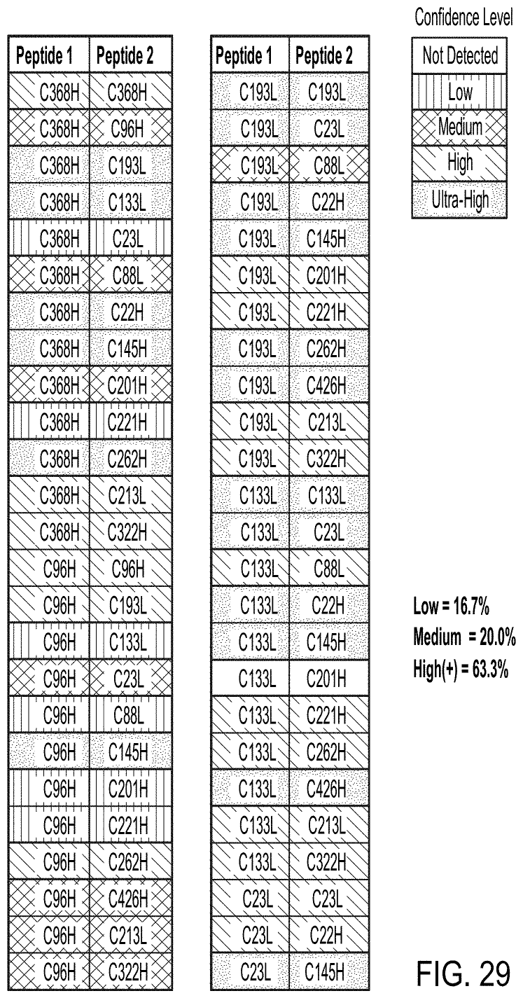

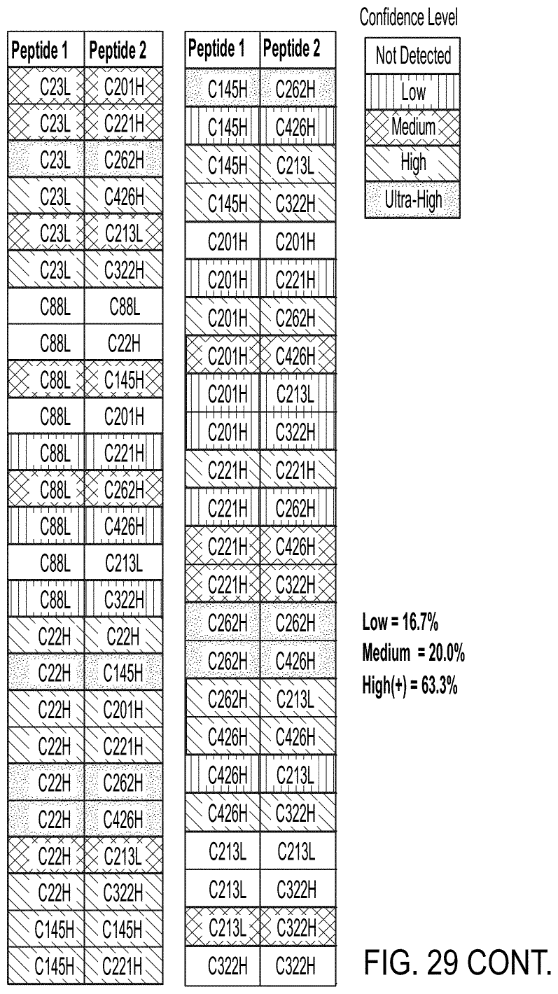

The results are depicted at FIG. 29, which shows all possible scrambled disulfide connections from mAb2, coded by confidence level. 63.3% of scrambled disulfide connections were identified with a high or ultra-high confidence level, 20% were identified with a medium confidence level, and 16.7% were identified with a low confidence level. Scrambled peptides corresponding to the hinge were omitted from the analysis. The data illustrated that the vast majority of all possible scrambled disulfides were identifiable to some extent. Again, the results raised the question of whether the disulfide scrambling could be artificially caused by the protocol used for trypsin digestion.

Accordingly, this issue was investigated in similar fashion as that discussed above for the mAb2 antibody. Specifically, for the mAb5 antibody, an experiment was performed to determine whether the amount of scrambled disulfides identified (see FIG. 29) was artificially caused by the choice of digestion procedure (e.g., higher pH digestion procedure). FIGS. 30A-30C each depict tables illustrating detected disulfides via the procedure discussed above that included the targeted MS2 approach combined with the confidence scoring system to assign confidence levels to each identified disulfide peptide fragment. Shown at FIGS. 30A-30C are all possible scrambled disulfide connections from mAb5, coded by confidence level in similar fashion as that discussed for FIG. 29, for each of the three different digestion procedures corresponding to the mAb4 procedure, the mAb5 procedure, and the low pH digestion kit procedure (refer to FIGS. 23A-23C). For each possible scrambled disulfide, the scrambling percentage was calculated as a function of the particular method of digestion (e.g., mAb4 procedure, mAb5 procedure, or low pH kit digestion procedure). The equation for calculating scrambling percentage is shown at FIG. 20D and was discussed above. As can be seen at FIGS. 30A-30C, all scrambled disulfides quantify to less than 0.01% when the low pH digestion procedure was used, as compared to higher percentages identified when the higher pH digestion procedures were used (e.g., mAb2 and mAb3 procedures). For some of the disulfides, there was interference, similar to that discussed above and shown exemplarily at FIG. 20E.

Taken together, the methodology discussed herein can effectively be used to identify low abundance scrambled disulfides in biomolecules, for example monoclonal antibodies.

These examples demonstrate that post-column TCEP partial reduction is a simple and effective way to identify scrambled disulfide peptides, via the generation of simpler MS/MS spectra (as compared to MS/MS spectra of intact disulfide peptides) and with all components sharing the exact same retention time. Further, the experiments show that digestion conditions under more basic (e.g., pH 7.5) conditions can induce artificial disulfide scrambling. Hence, an abundance of artificial scrambled disulfide peptides may depend on digestion protocol, and free thiols likely contribute to, but are not solely responsible for, disulfide scrambling. Performing non-reduced digestion under more acidic conditions (e.g., pH 5.7) can prevent artificial disulfide scrambling. Finally, the mAbs discussed herein (e.g., mAb2 and mAb5) were shown to contain negligible levels of real scrambled disulfides.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

1. A method for identification of one or more non-native disulfide bonds in a biomolecule, comprising:
performing a digestion of the biomolecule under non-reducing conditions to yield a sample that includes a plurality of fragments of the biomolecule;
contacting the sample to a separation column under conditions that permit sample components to bind to a column substrate;
applying a first mobile phase gradient to the separation column, wherein the first mobile phase gradient comprises trifluoroacetic acid (TFA) and a small molecule additive at a concentration of about 1-2 mM;
applying a second mobile phase gradient to the separation column, wherein the second mobile phase gradient comprises TFA in acetonitrile (ACN) and a small molecule additive at the concentration of about 1-2 mM;
performing a partial reduction procedure via treatment of eluted sample components with tris(2-carboxyethyl)phosphine (TCEP) at a concentration of 10-100 μM;
applying the partially reduced eluted sample components to a mass spectrometer; and
performing a mass spectrometric analysis on the partially reduced eluted sample components to identify the one or more non-native disulfide bonds in the biomolecule.

2. The method of claim 1, wherein the small molecule additive in the first mobile phase and/or the small molecule additive in the second mobile phase is selected from glycine, alanine, serine, valine, N-acetyl glycine, methionine, β-alanine, aspartic acid, or N-methyl glycine.

3-5. (canceled)

6. The method of claim 1, wherein TFA concentration in the first mobile phase is about 0.05% to 0.1% TFA in H₂O; and
wherein TFA concentration in the second mobile phase comprises about 0.05% TFA in 80% ACN and 20% H₂O or about 0.1% TFA in 80% ACN and 20% H₂O.

7-8. (canceled)

9. The method of claim 1, wherein performing the digestion of the biomolecule further comprises:
performing a denaturation and alkylation step to yield a denatured alkylated biomolecule;
performing a pre-digestion step on the denatured alkylated biomolecule to yield a predigested denatured alkylated biomolecule; and
performing a digestion step on the predigested denatured alkylated biomolecule following the pre-digestion step to yield the sample that is contacted to the separation column.

10. The method of claim 9, wherein:
(a) the denaturation and alkylation step includes denaturing the biomolecule in 7-9M urea in the presence of an alkylating agent at a pH of about 5.5-5.9;
(b) the denaturation and alkylation step includes denaturing the biomolecule in 7-9M urea in the presence of an alkylating agent at a pH of about 5.5-5.9, and performing the denaturation and alkylation step between 45-55° C.;
(c) the denaturation and alkylation step includes denaturing the biomolecule in 7-9M urea in the presence of an alkylating agent at a pH of about 5.5-5.9, wherein the alkylating agent is N-ethyl maleimide (NEM) at a concentration between 5-15 mM
(d) the denaturation and alkylation step includes denaturing the biomolecule in 7-9M urea in the presence of an alkylating agent at a pH of about 5.5-5.9, wherein the alkylating agent is iodo-acetamide (IAM) at a concentration of about 0.5-5 mM;
(e) the denaturation and alkylation step includes denaturing the biomolecule in 7-9M urea in the presence of an alkylating agent at a pH of about 5.5-5.9, and performing the denaturation and alkylation step for 20-40 minutes;
(f) performing the pre-digestion step further comprises incubating the denatured alkylated biomolecule in the presence of recombinant Lys-C protease at a pH between 5-5.6;
(g) wherein the method further comprises performing the pre-digestion step at between 35-40° C.;
(h) wherein the method further comprises performing the pre-digestion step for 30 minutes to 90 minutes; and/or
(i) performing the pre-digestion step further comprises incubating the denatured alkylated biomolecule in the presence of recombinant Lys-C protease at a pH between 5-5.6, wherein a ratio of recombinant Lys-C protease to the denatured alkylated biomolecule is between 1:5 and 1:20, respectively.

11-18. (canceled)

19. The method of claim 9, wherein:
(a) performing the digestion step further comprises incubating the predigested denatured alkylated biomolecule in the presence of recombinant Lys-C protease and trypsin protease at a pH between 5-5.6;
(b) a ratio of recombinant Lys-C protease to the predigested denatured alkylated biomolecule during the digestion step is about between 1:5 and 1:20, respectively;
(c) a ratio of trypsin protease to the predigested denatured alkylated biomolecule is between 1:2 and 1:10, respectively;
(d) the method further comprises performing the digestion step between 35-40° C.; and/or
(e) the method further comprises performing the digestion step for 2-4 hours.

20-23. (canceled)

24. The method of claim 1, wherein the partial reduction procedure is conducted for a duration of 500 ms-3 s; and/or
wherein the partially reduced eluted sample components include one or more disulfide peptides and corresponding reduced partner peptides.

25. (canceled)

26. The method of claim 24, wherein each of the one or more disulfide peptides and corresponding reduced partner peptides enter into the mass spectrometer at a same time, and/or the mass spectrometer is a tandem mass spectrometer; and
wherein performing the mass spectrometric analysis includes obtaining a MS1 spectra and a MS2 spectra.

27. The method of claim 26, further comprising building a parallel reaction monitoring (PRM) inclusion list with the corresponding reduced partner peptides, and assigning a disulfide identification confidence score for the one or more disulfide peptides based on a confidence scoring system.

28. (canceled)

29. The method of claim 27, wherein the confidence scoring system further comprises:
indicating whether a MS1 mass of a disulfide peptide is identified via the mass spectrometric analysis;
indicating whether a MS1 mass of a first reduced partner peptide corresponding to the disulfide peptide is identified via the mass spectrometric analysis;
indicating whether a MS1 mass of a second reduced partner peptide corresponding to the disulfide peptide is identified via the mass spectrometric analysis;
indicating whether a MS2 mass of the first reduced partner peptide is identified with a score greater than a predetermined threshold;
indicating whether a MS2 mass of the second reduced partner peptide is identified with a score greater than the predetermined threshold;
for each of the indicating steps of the confidence scoring system, assigning a single point where the corresponding peptide is identified and no points where the corresponding peptide is not identified;
summing the single points; and
assigning the disulfide identification confidence score based on the summing, where the greater the sum, the higher the confidence.

30. The method of claim 1, wherein performing the mass spectrometric analysis further comprises:
determining a disulfide scrambling percentage for each of the one or more non-native disulfide bonds in the biomolecule;
wherein the disulfide scrambling percentage is a ratio of an average peak area of a peptide that includes a non-native disulfide bond to a sum of the average peak area of the peptide that includes the non-native disulfide bond plus another average peak area of two peptides that include native disulfide bonds corresponding to cysteine residues that are involved in the non-native disulfide bond.

31. The method of claim 1, wherein the concentration of TCEP is between 20 μM and 80 μM.

32. (canceled)

33. The method of claim 1, wherein the biomolecule is a monoclonal antibody; and
wherein the monoclonal antibody is of isotype IgG1, IgG2, IgG3, IgG4, or mixed isotype.

34. (canceled)

35. A method of identifying one or more scrambled disulfide bonds in a biomolecule, comprising:
performing a digestion of the biomolecule under non-reducing conditions and at an acidic pH to yield a sample that includes one or more disulfide peptides;
contacting the sample to a separation column to separate components of the sample, wherein separation of the components is performed in the presence of glycine at a concentration of 1-2 mM;
partially reducing eluted sample components following separation via the separation column; and
performing a mass spectrometric analysis via a tandem mass spectrometer on the partially reduced eluted sample components that includes via a first mass analyzer, identifying a MS1 mass of each of the one or more disulfide peptides and/or corresponding reduced partner peptides, and via a second mass analyzer, identifying just a MS2 mass of one or more of the corresponding reduced partner peptides by targeting just the corresponding reduced partner peptides and not each of the one or more disulfide peptides.

36. The method of claim 35, wherein targeting just the corresponding reduced partner peptides and not each of the one or more disulfide peptides further comprises:
building a parallel reaction monitoring inclusion list with just the corresponding reduced partner peptides; and
assigning a disulfide identification confidence score for each of the one or more disulfide peptides.

37. (canceled)

38. The method of claim 36, wherein assigning the disulfide identification confidence score is based on a disulfide confidence scoring system that includes assigning a point for each MS1 mass that is identifiable and a point for each identifiable MS2 mass above a predetermined threshold detection level, for a particular disulfide peptide and corresponding reduced partner peptides; and
summing the points to obtain the disulfide identification confidence score, where a maximum score is 5 corresponding to a highest confidence score.

39. The method of claim 35, wherein performing the mass spectrometric analysis further comprises:
determining a disulfide scrambling percentage for each of the one or more scrambled disulfide bonds in the biomolecule;
wherein the disulfide scrambling percentage is a ratio of an average peak area of a peptide that includes a scrambled disulfide bond to a sum of the average peak area of the peptide that includes the scrambled disulfide bond plus another average peak area of two peptides that include native disulfide bonds corresponding to cysteine residues that are involved in the scrambled disulfide bond.

40. The method of claim 35, wherein:
(a) partially reducing eluted sample components following separation via the separation column is via treatment of eluted sample components with tris(2-carboxyethyl)phosphine (TCEP) at a concentration of 10-100 μM;
(b) the method further comprises partially reducing eluted sample components for a time period of about 1-3 seconds; and/or
(c) partially reducing eluted sample components occurs at a reduction efficiency of about 1-3%.

41-44. (canceled)

45. The method of claim 35, wherein the separation column is a reverse-phase high performance liquid chromatography column or a hydrophilic interaction liquid chromatography (HILIC) column.

46. (canceled)

47. The method of claim 35, wherein the acidic pH at which digestion of the biomolecule is performed is between 5-5.6; and
wherein separation of the components of the sample on the separation column is performed in the presence of 0.05-0.1% trifluoroacetic acid (TFA) and an absence of NH₄OH.

48. (canceled)

49. The method of claim 35, wherein performing the digestion includes incubating the biomolecule in the presence of recombinant Lys-C protease and trypsin protease.

50. The method of claim 35, further comprising prior to performing the digestion, denaturing the biomolecule in the presence of one or more alkylating agents at a pH between 5.5 and 5.9.

51. The method of claim 35, wherein the biomolecule is a monoclonal antibody; and wherein the monoclonal antibody is of isotype IgG1, IgG2, IgG3, IgG4, or mixed isotype.


SEQUENCE LISTING (47 sequences)

SEQ ID NO: 1 - protein, 12 aa, Artificial Sequence
  peptide of mAb2
GPSVFPLAPCSR

SEQ ID NO: 2 - protein, 14 aa, Artificial Sequence
  peptide of mAb2
TYTCNVDHKPSNTK

SEQ ID NO: 3 - protein, 16 aa, Artificial Sequence
  Peptide of mAb1
VVSVLTVLHQDWLNGK

SEQ ID NO: 4 - protein, 7 aa, Artificial Sequence
  Peptide of mAb1
DTLMISR

SEQ ID NO: 5 - protein, 10 aa, Artificial Sequence
  Peptide of mAb1
NQVSLTCLVK

SEQ ID NO: 6 - protein, 23 aa, Artificial Sequence
  Peptide of mAb1
WQQGNVFSCSVMHEALHNHYTQK

SEQ ID NO: 7 - protein, 14 aa, Artificial Sequence
  Peptide of mAb2
STSESTAALGCLVK

SEQ ID NO: 8 - protein, 11 aa, Artificial Sequence
  Peptide of mAb2
LSCAGSGFTFR

SEQ ID NO: 9 - protein, 11 aa, Artificial Sequence
  Peptide of mAb2
AEDTAVYYCAK

SEQ ID NO: 10 - protein, 33 aa, Artificial Sequence
  Peptide of mAb2
TPEVTCVVVDVSQEDPEVQFNWYVDGVEVHNAK

SEQ ID NO: 11 - protein, 23 aa, Artificial Sequence
  Peptide of mAb2
WQEGNVFSCSVMHEALHNHYTQK

SEQ ID NO: 12 - protein, 24 aa, Artificial Sequence
  Peptide of mAb2
DIVMTQSPLSLPVTPGEPASISCR

SEQ ID NO: 13 - protein, 26 aa, Artificial Sequence
  Peptide of mAb2
VEAEDVGFYYCMQALQTPYTFGQGTK

SEQ ID NO: 14 - protein, 16 aa, Artificial Sequence
  Peptide of mAb2
SGTASVVCLLNNFYPR

SEQ ID NO: 15 - protein, 17 aa, Artificial Sequence
  Peptide of mAb2
VYACEVTHQGLSSPVTK

SEQ ID NO: 16 - protein, 7 aa, Artificial Sequence
  Peptide of mAb2
SFNRGEC

SEQ ID NO: 17 - protein, 8 aa, Artificial Sequence
  Peptide of mAb2
DYAMTWVR

SEQ ID NO: 18 - protein, 15 aa, Artificial Sequence
  Peptide of mAb2
SGQSPQLLIYLGSNR

SEQ ID NO: 19 - protein, 49 aa, Artificial Sequence
  Peptide of mAb2
DYFPEPVTVSWNSGALTSGVHTFPAVLQSSGLYSLSSVVTVPSSSLGTK

SEQ ID NO: 20 - protein, 5 aa, Artificial Sequence
  Peptide of mAb2
ADYEK

SEQ ID NO: 21 - protein, 22 aa, Artificial Sequence
  Peptide of mAb2
GFYPSDIAVEWESNGQPENNYK

SEQ ID NO: 22 - protein, 17 aa, Artificial Sequence
  Peptide of mAb2
TTPPVLDSDGSFFLYSR

SEQ ID NO: 23 - protein, 19 aa, Artificial Sequence
  Peptide of mAb5
EVQLVESGGGLVQPGGSLR

SEQ ID NO: 24 - protein, 18 aa, Artificial Sequence
  Peptide of mAb5
DIQMTQSPSSLSASVGDR

SEQ ID NO: 25 - protein, 12 aa, Artificial Sequence
  Peptide of mAb5
GPSVFPLAPSSK

SEQ ID NO: 26 - protein, 14 aa, Artificial Sequence
  Peptide of mAb5
FNWYVDGVEVHNAK

SEQ ID NO: 27 - protein, 8 aa, Artificial Sequence
  Peptide of mAb5
ALPAPIEK

SEQ ID NO: 28 - protein, 5 aa, Artificial Sequence
  Peptide of mAb5
DELTK

SEQ ID NO: 29 - protein, 17 aa, Artificial Sequence
  Peptide of mAb5
TTPPVLDSDGSFFLYSK

SEQ ID NO: 30 - protein, 19 aa, Artificial Sequence
  Peptide of mAb5
LSCAASGFTSSSYAMNWVR

SEQ ID NO: 31 - protein, 14 aa, Artificial Sequence
  Peptide of mAb5
STSGGTAALGCLVK

SEQ ID NO: 32 - protein, 63 aa, Artificial Sequence
  Peptide of mAb5
DYFPEPVTVSWNSGALTSGVHTFPAVLQSSGLYSLSSVVTVPSSSLGTQTYICNVNHKPSNTK

SEQ ID NO: 33 - protein, 4 aa, Artificial Sequence
  Peptide of mAb5
SCDK

SEQ ID NO: 34 - protein, 19 aa, Artificial Sequence
  Peptide of mAb5
TPEVTCVVVDVSHEDPEVK

SEQ ID NO: 35 - protein, 6 aa, Artificial Sequence
  Peptide of mAb5
VTITCR

SEQ ID NO: 36 - protein, 41 aa, Artificial Sequence
  Peptide of mAb5
FSGSGSGTDFTLTISSLQPEDFATYYCQQSYSTLTFGQGTR

SEQ ID NO: 37 - protein, 10 aa, Artificial Sequence
  Peptide of mAb2
NQVSLTCLVK

SEQ ID NO: 38 - protein, 16 aa, Artificial Sequence
  Peptide of mAb2
VVSVLTVLHQDWLNGK

SEQ ID NO: 39 - protein, 7 aa, Artificial Sequence
  Peptide of mAb2
DTLMISR

SEQ ID NO: 40 - protein, 5 aa, Artificial Sequence
  Peptide of mAb5
ADYEK

SEQ ID NO: 41 - protein, 11 aa, Artificial Sequence
  Peptide of mAb5
AEDTAVYYCAK

SEQ ID NO: 42 - protein, 10 aa, Artificial Sequence
  Peptide of mAb5
NQVSLTCLVK

SEQ ID NO: 43 - protein, 23 aa, Artificial Sequence
  Peptide of mAb5
WQQGNVFSCSVMHEALHNHYTQK

SEQ ID NO: 44 - protein, 16 aa, Artificial Sequence
  Peptide of mAb5
SGTASVVCLLNNFYPR

SEQ ID NO: 45 - protein, 17 aa, Artificial Sequence
  Peptide of mAb5
VYACEVTHQGLSSPVTK

SEQ ID NO: 46 - protein, 27 aa, Artificial Sequence
  Peptide of mAb2
YGPPCPPCPAPEFLGGPSVFLFPPKPK

SEQ ID NO: 47 - protein, 26 aa, Artificial Sequence
  Peptide of mAb5
THTCPPCPAPELLGGPSVFLFPPKPK